SEQUENCE LISTING (104 sequences)

SEQ ID NO: 1 - DNA, 5250 bp, Artificial sequence
  pLP
gatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcgcgttggccgattcattaatgcagctggcacgacaggtttcccgactggaagtagtgccccaactggggtaacctttgagttctctcagttgggggcgtagttgatcttttctacgggcaggacggtgatagggataacagggtaatgtaccatttacgttgacaccacctttcgcgtatggcgtgatagcgcccggaagagagtcaattcagggtggtgaatatgaatagttcgacaaagatcgcattggtaattacgttgctcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcaaattcggtgtacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcgaatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcaatgaccttcatgttgacccctcaagctcaggggagtaaacaggagacaagtgcttagtagggataacagggtaatgatggcgcctcatccctgaagccaaggcatcaaataaaacgaaaggctcagtcgaaagactgggcctttcgttttatctgttgtttgtcggtgaacgctctcctgagtaggacaaatccgccgccctagacctaggggatatattccgctaaatcactagtgcggccgcctgcagtagtgccccaactggggtaacctccgagttctctcagttgggggcgtaggtgtaggctggagctgcttcgaagttcctatactttctagagaataggaacttcggaataggaacttcaagatcccccacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatgcatgaccaaaatcccttaacgtgagttttcgttccactgagcgtcagacttcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtctt

SEQ ID NO: 2 - DNA, 50 bp, Artificial sequence
  Homolgy 1 to djlA_yabP
ctcaatgcacggtttacgggaggggttctgtaggttttatcgcgttgacc

SEQ ID NO: 3 - DNA, 50 bp, Artificial sequence
  Homology 2 to djlA_yabP
agacgtaaaaatataattccgctcgtcgtaaagctctcaaccttaagcag

SEQ ID NO: 4 - DNA, 50 bp, Artificial sequence
  Homlogy 1 to ylcI_nohD
tagatgataattattatcattttgtgggtcctttccggcgatccgacagg

SEQ ID NO: 5 - DNA, 50 bp, Artificial sequence
  homology 2 to ylcI_nohD
ccggaaaattttcataaatagcgaaaacccgcgaggtcgccgccccgtaa

SEQ ID NO: 6 - DNA, 50 bp, Artificial sequence
  Homology 1 to tyrV_tyrT
ttcgtcgcttcgctcctcacccttcgggccgttgcctgtggcaacgttct

SEQ ID NO: 7 - DNA, 50 bp, Artificial sequence
  Homology 2 to tyrV_tyrT
cggggaagggtgagaaccttcgactaaggttcgattcgagcgaaagcgag

SEQ ID NO: 8 - DNA, 50 bp, Artificial sequence
  Homology 1 to ypjC_ileY
agtagtagatgtttaaggcgtggcagagacatttcatccttactctacgg

SEQ ID NO: 9 - DNA, 50 bp, Artificial sequence
  Homology 2 to ypjC_ileY
tcgctcactgatgataagtgagtaccacaaccaatgtatgtagaacaatg

SEQ ID NO: 10 - DNA, 50 bp, Artificial sequence
  Homology 11 to yhiM_yhiN
cagcaaagttactgtttttttcaacctgttcatatttcataaagatctgg

SEQ ID NO: 11 - DNA, 50 bp, Artificial sequence
  Homology 2 to yhiM_yhiN
catgcttaatataaggtggatggaaaggtgattgaaaactcactcagtgg

SEQ ID NO: 12 - DNA, 50 bp, Artificial sequence
  Homology 1 to thrW_ykfN
tcttaatgtaacagctggtgtaagtaaattctatcaacgaagatcaatct

SEQ ID NO: 13 - DNA, 50 bp, Artificial sequence
  Homology 2 to thrW_ykfN
aaggatgtatagtgagcgaagccctatcaggcctttttggtcagtagata

SEQ ID NO: 14 - DNA, 50 bp, Artificial sequence
  Homology 1 to entF_fepE
ttgatttataggtttgatgaatatttctcttaaatagagtgaatgttgca

SEQ ID NO: 15 - DNA, 50 bp, Artificial sequence
  Homology 2 to entF_fepE
agttggtgataattatccgaagctgaagtttgtaaattccttccactgaa

SEQ ID NO: 16 - DNA, 50 bp, Artificial sequence
  Homology 1 to ydaG_racR
accactgcctggtaactcgaagtattgcccggcgttctgtggggcggggt

SEQ ID NO: 17 - DNA, 50 bp, Artificial sequence
  homolgy 2 to ydaG_racR
agcctattgacaatcaattaggcattacctatagttccagcataccaccc

SEQ ID NO: 18 - DNA, 50 bp, Artificial sequence
  Homology to ileY_ygaQ
gtatctaataatataactttattacattagctgaagagttttcgcatcat

SEQ ID NO: 19 - DNA, 50 bp, Artificial sequence
  Homolgy 2 to ileY_ygaQ
attgctatacgaagtttatttttatggagtgaaaagtaacagatatcata

SEQ ID NO: 20 - DNA, 50 bp, Artificial sequence
  Homolgy 1 to dinD_yicG
ttttccccctcagttttaacctattttttcttatgcattttctcagacaa

SEQ ID NO: 21 - DNA, 50 bp, Artificial sequence
  Homology 2 to dinD_yicG
gttatgtgaaatcgctattttctgtagcagagatgcattcttctgacttc

SEQ ID NO: 22 - DNA, 50 bp, Artificial sequence
  Homology 1 to ykfA_perR
aggcagctgcgcgactgctggctcaggcaatgaatgagttataatagcag

SEQ ID NO: 23 - DNA, 50 bp, Artificial sequence
  Homology 2 to ykfA_perR
aaggtgtatcacggcggctcatactctcaataaatccctgttagtaaatg

SEQ ID NO: 24 - DNA, 50 bp, Artificial sequence
  Homology 1 to ybfK_kdpE
caataaaaaatgatcaatcttaatttatttaatgatgagctttttactca

SEQ ID NO: 25 - DNA, 50 bp, Artificial sequence
  Homology 2 to ybfK_kdpE
tttttatcttaaacaacacacaaaaataacaattcaatattttatattac

SEQ ID NO: 26 - DNA, 50 bp, Artificial sequence
  Homology 1 to cspF_quuQ
gtttagggacattgtactggaagaaaacattttaaacatcaggcaaataa

SEQ ID NO: 27 - DNA, 50 bp, Artificial sequence
  Homology 2 to cspF_quuQ
ctcatcccgggactcatgtctgttaacttattatttagctggtgacttgg

SEQ ID NO: 28 - DNA, 50 bp, Artificial sequence
  Homology 1 to yqaB_argQ
tggataaaggagttatttagaaatgagatatttttgaaggaaattttttg

SEQ ID NO: 29 - DNA, 50 bp, Artificial sequence
  Homology 2 to yqaB_argQ
cccgaagggcgaacgtcagtgagtcatcctcccggatgcaccatcttctc

SEQ ID NO: 30 - DNA, 50 bp, Artificial sequence
  Homology 1 to frvA_rhaM
tgaaaggtcagatttgcggagtaatgcacataatggttatttaaataaac

SEQ ID NO: 31 - DNA, 50 bp, Artificial sequence
  Homology 2 to frvA_rhaM
attgtgagtaaatcacaaaaataatgaataacccattaatgattcatgtg

SEQ ID NO: 32 - DNA, 50 bp, Artificial sequence
  Homology to insN_eyeA
tgcccgcagggtgatgtaacccgctgacaacggggattgaggcgagatca

SEQ ID NO: 33 - DNA, 50 bp, Artificial sequence
  Homology 2 to insN_eyeA
catgttcttcaacctttcagtacttaaccttgaggatcatctcggcttag

SEQ ID NO: 34 - DNA, 50 bp, Artificial sequence
  Homology 1 to ybfC_ybfQ
tttattttgcgttccatttgcagggaaagatcacgtaacgctactttttt

SEQ ID NO: 35 - DNA, 50 bp, Artificial sequence
  Homology 2 to ybfC_ybfQ
caataagtagtatctcaattgttgaacttaaaattcgaattatttagtac

SEQ ID NO: 36 - DNA, 50 bp, Artificial sequence
  Homology 1 to rseX_yedS
attttcatgaatatttatatttagaattcataattatgaattatattaaa

SEQ ID NO: 37 - DNA, 50 bp, Artificial sequence
  Homology 2 to rseX_yedS
gattacatgtaacaaatgtatttaaaagatatcaaaatgtttctaatcta

SEQ ID NO: 38 - DNA, 50 bp, Artificial sequence
  Homology 1 to ygcE_queE
ggtggtttatccccgctggcgcggggaactcgacagaacggcctcagtag

SEQ ID NO: 39 - DNA, 50 bp, Artificial sequence
  Homology 2 to ygcE_queE
gaaaacaggtgttccccgcgccagcggggataaaccggagcctgacgaga

SEQ ID NO: 40 - DNA, 50 bp, Artificial sequence
  Homology 1 to frwA_frwC
caatttgcgacgcgtctcacaagacgctgttttgcggcatgcttccggtt

SEQ ID NO: 41 - DNA, 50 bp, Artificial sequence
  Homology 2 to frwA_frwC
acttttgtaatatcagtacaaaaatgcgatccgcctcataacttgcgata

SEQ ID NO: 42 - DNA, 50 bp, Artificial sequence
  Homology 1 to ykgA_ykgQ
ccgaaaatagagaggtttcagtcctacattattaatgaattttttgcata

SEQ ID NO: 43 - DNA, 50 bp, Artificial sequence
  Homology 2 to ykgA_ykgQ
gtctacgttaaaacgtaacctcaaagtagtatgtggattttgatatcact

SEQ ID NO: 44 - DNA, 50 bp, Artificial sequence
  Homology 1 to ybiJ_ybiI
aaatcgaagagaattgaccgccttgttcaaataaattgattgatatctaa

SEQ ID NO: 45 - DNA, 50 bp, Artificial sequence
  Homology 2 to ybiJ_ybiI
cggtataaaacaagttcataagtacaacaaataaatggtttatcagtagg

SEQ ID NO: 46 - DNA, 50 bp, Artificial sequence
  Homology 1 to yeeJ_yeeL
cacagaaaatgaataaataaaaatgcggcaccgccagaatcgcgttcgat

SEQ ID NO: 47 - DNA, 50 bp, Artificial sequence
  Homology 2 to yeeJ_yeeL
aaaccagcctttagatcaaagcagtactcaccgaaaatgatcatagtcac

SEQ ID NO: 48 - DNA, 50 bp, Artificial sequence
  Homology 1 to ygeF_ygeG
gatgttattagtttgtagtgaacagtacttttaccaataatgaaaaatat

SEQ ID NO: 49 - DNA, 50 bp, Artificial sequence
  Homology 2 to ygeF_ygeG
tatatttatcttttttaaattatgagttttaagcttgcattgcttatggt

SEQ ID NO: 50 - DNA, 50 bp, Artificial sequence
  Homology 1 to malM_yjbI
tccttcctgggatatgagcgattttttatagtaactcacttcttcttcac

SEQ ID NO: 51 - DNA, 50 bp, Artificial sequence
  Homology 2 to malM_yjbI
gcgaaaggaaaagaatctctgataaggcattgagataatggatattctta

SEQ ID NO: 52 - DNA, 50 bp, Artificial sequence
  Homology 1 to ykgH_betA
aggaatgttcgggttaaatatcagcaaaaagcccgcatcatgaatactgg

SEQ ID NO: 53 - DNA, 50 bp, Artificial sequence
  Homology 2 to ykgH_betA
gggggaccgaatccttatataaacactgaggtaactctcatgcttcatat

SEQ ID NO: 54 - DNA, 50 bp, Artificial sequence
  Homology 1 to ymgF_ycgH
gcaactattaacaattttgatgtcgaagagttatttgttaaacaaaatcg

SEQ ID NO: 55 - DNA, 50 bp, Artificial sequence
  Homology 2 to ymgF_ycgH
gcattatcatttttcaccttattttcatgacattgatcactttgaggtga

SEQ ID NO: 56 - DNA, 50 bp, Artificial sequence
  Homology to udk_yegE
cgcgctcagagttaattgttgacaaagaattcccgggggcaaattacgtt

SEQ ID NO: 57 - DNA, 50 bp, Artificial sequence
  Homology 2 to udk_yegE
ataatttgcgcaactgcgtttaacattttttaccttacataaaactgatc

SEQ ID NO: 58 - DNA, 50 bp, Artificial sequence
  Homology 1 to ygeK_ygeN
attataagcaaaatccaaagaatacattgatgaaataataatgaaatata

SEQ ID NO: 59 - DNA, 50 bp, Artificial sequence
  Homology 2 to ygeK_ygeN
gattttttaatgcctgtggtatttttttacgcaaaaattttatttttaat

SEQ ID NO: 60 - DNA, 50 bp, Artificial sequence
  Homology 1 to yjcS_alsK
ttgcgactttaataagtggaagtgtgagcggaacgcgccattttattagg

SEQ ID NO: 61 - DNA, 50 bp, Artificial sequence
  Homology 2 to yjcS_alsK
attttctgcaatgatagttttactgtaattttccctcttcagcacaaatg

SEQ ID NO: 62 - DNA, 50 bp, Artificial sequence
  Homology 1 to yahK_yahL
cgaaataatatcaaagtagcagtaaaacctataacgtaaatttaaattgt

SEQ ID NO: 63 - DNA, 50 bp, Artificial sequence
  Homology 2 to yahK_yahL
tcgctcataactaacgtgtgaagtattgtgtactggagggcgttaattta

SEQ ID NO: 64 - DNA, 50 bp, Artificial sequence
  Homology 1 to dadX_cvrA
aacctgaactcaccgcacaggcgttctacataaaacgcttacgcttcatt

SEQ ID NO: 65 - DNA, 50 bp, Artificial sequence
  Homology 2 to dadX_cvrA
gctccatcaagggtaaagcgtgatttatctgaagtcgagttcgagtcaac

SEQ ID NO: 66 - DNA, 50 bp, Artificial sequence
  Homology 1 to yffL_yffM
tttttagcctcccggtcggtcatagagagtcgcctagagttaaacagaag

SEQ ID NO: 67 - DNA, 50 bp, Artificial sequence
  Homology 2 to yffL_yffM
agcatggttaatgctcgcaaccagccgacctatcaggcggcgaaataatt

SEQ ID NO: 68 - DNA, 50 bp, Artificial sequence
  Homology 1 to sibD_sibE
aaaagccggggattttttatatctgcgttccgctaaaaggtgcaaatgct

SEQ ID NO: 69 - DNA, 50 bp, Artificial sequence
  Homology 2 to sibD_sibE
aggcaattttgccttccccgagcggtcacgcaaaacgctgcaacgtcctg

SEQ ID NO: 70 - DNA, 50 bp, Artificial sequence
  homology 1 to yjhV_fecE
cctgaaatctaaacttagtcatgtcacgtttttgggtttctaaaatttta

SEQ ID NO: 71 - DNA, 50 bp, Artificial sequence
  Homology 2 to yjhV_fecE
gcttaacggacatttctgtataacccttacggcaacgaaaaacgcgaagt

SEQ ID NO: 72 - DNA, 50 bp, Artificial sequence
  Homology 1 to yfjQ_yfjR
tcgtgtgcctcaatcccccggttatagcttttaacccccgttacatctgg

SEQ ID NO: 73 - DNA, 50 bp, Artificial sequence
  Homology 2 to yfjQ_yfjR
ggcggacagggtatggacaacgcagaaactattttttatttctgcaaaag

SEQ ID NO: 74 - DNA, 50 bp, Artificial sequence
  Homology 1 to glpD_yzgL
aggcctacgtggtttatgcaatatattgaatttgcatggtcttgtaggcc

SEQ ID NO: 75 - DNA, 50 bp, Artificial sequence
  Homology 2 to glpD_yzgL
ttgacaaagtgcgctttgttcatgccggatgcgacgtgaacgtcttatct

SEQ ID NO: 76 - DNA, 50 bp, Artificial sequence
  Homology 1 to yjiP_yjiR
tattgaactttaaagatttttgtagacctggtcaggcgttcacatggcat

SEQ ID NO: 77 - DNA, 50 bp, Artificial sequence
  Homology 2 to yjiP_yjiR
atcgccacgttccagcctgaattaagcaaagtacgctttgttcatgccgg

SEQ ID NO: 78 - DNA, 50 bp, Artificial sequence
  Homology 1 to lacZ_lacI
ccgagttaacgccatcaaaaataattcgcgtctggccttcctgtagccag

SEQ ID NO: 79 - DNA, 50 bp, Artificial sequence
  Homology 2 to lacZ_lacI
cattaatgcagctggcacgacaggtttcccgactggaaagcgggcagtga

SEQ ID NO: 80 - DNA, 50 bp, Artificial sequence
  Homology 1 to ycbW_ycbX
tgaaaccgcaggttaatgttgacagcttcagcctcgaacaggcagtctaa

SEQ ID NO: 81 - DNA, 50 bp, Artificial sequence
  Homology 2 to ycbW_ycbX
ttctttgctgtagctgtgtaccgaagactgcacttaagttggcgcgttag

SEQ ID NO: 82 - DNA, 50 bp, Artificial sequence
  Homology 1 to nupG_speC
ataaacacgttcgtgtcccgacaggcacacagacggttagccactaatta

SEQ ID NO: 83 - DNA, 50 bp, Artificial sequence
  Homology 2 to nupG_speC
gtaagaataaaaaaaacgggtcaccttctggcgacccgtttttctttgcg

SEQ ID NO: 84 - DNA, 50 bp, Artificial sequence
  Homology 1 to aslB_aslA
tgtaggctggataagatgcgtcagcatcgcatccggcaaaggcagatctc

SEQ ID NO: 85 - DNA, 50 bp, Artificial sequence
  Homology 2 to aslB_aslA
aatatccaccacgcgcgcagattaaatctgactaagccggcgctatcgct

SEQ ID NO: 86 - DNA, 50 bp, Artificial sequence
  homology 1 to atpI_gidB
caaaaagcggtcaaattatacggtgcgcccccgtgatttcaaacaataag

SEQ ID NO: 87 - DNA, 50 bp, Artificial sequence
  Homology 2 to atpI_gidB
ataacgtggctttttttggtaagcagaaaataagtcattagtgaaaatat

SEQ ID NO: 88 - DNA, 50 bp, Artificial sequence
  Homology 1 to yieN_trkD
tggcgtcctttcgtcaaaagttctgcgtaaattgcgagtatagacgtttc

SEQ ID NO: 89 - DNA, 50 bp, Artificial sequence
  Homology 2 to yieN_trkD
gtatgcacgattaacggcaaaatcgtactcctaaatgcggccacattaac

SEQ ID NO: 90 - DNA, 50 bp, Artificial sequence
  homology 1 to ybbD_ylbI
ctgagaaaagacatgtcggctattgtgtaaagccatatagctcagacgat

SEQ ID NO: 91 - DNA, 50 bp, Artificial sequence
  Homology 2 to ybbD_ylbI
ttctatgtaaactctctgactgttcattttatttgttgtttcagggtcgg

SEQ ID NO: 92 - DNA, 50 bp, Artificial sequence
  Homology 1 to essQ_cspB
atggtgcaatatgtttgaaaagatcggagtctacggggtagttttgacag

SEQ ID NO: 93 - DNA, 50 bp, Artificial sequence
  Homology 2 to essQ_cspB
gataattacggcgtgattttgagtttttacgttctgacataggcttttcc

SEQ ID NO: 94 - DNA, 50 bp, Artificial sequence
  Homology 1 to nth_ydgR
ttaacgtcaatgatgccattgcttagcgttatcatcaggtaatccgtttg

SEQ ID NO: 95 - DNA, 50 bp, Artificial sequence
  Homology 2 to nth_ydgR
gatagtccagtttctgaaaaatagccagtgtaatgttttgtaggtcaata

SEQ ID NO: 96 - DNA, 50 bp, Artificial sequence
  Homology 1 to ackA_pta
ctatggctccctgacgtttttttagccacgtatcaattataggtacttcc

SEQ ID NO: 97 - DNA, 50 bp, Artificial sequence
  homology 2 to ackA_pta
ttatttccggttcagatatccgcagcgcaaagctgcggatgatgacgaga

SEQ ID NO: 98 - DNA, 50 bp, Artificial sequence
  Homology 1 to fucl_fucK
ttactccctgatgtgatgcccggtcgctccggctaccgggcctgaacaag

SEQ ID NO: 99 - DNA, 50 bp, Artificial sequence
  Homology 2 to fucl_fucK
gctcctgcaatatagccggataacattgcttatccggctaaccactcttg

SEQ ID NO: 100 - DNA, 50 bp, Artificial sequence
  Homology 1 to xylB_xylA
tatcccgatatacatatcgatcgttccttaaaaaaatgcccggtatcgct

SEQ ID NO: 101 - DNA, 50 bp, Artificial sequence
  Homology 2 to xylB_xylA
tgttcgacaaataacggctaactgtgcagtccgttggcccggttatcggt

SEQ ID NO: 102 - DNA, 7453 bp, Artificial sequence
  p2a_25_10-5_lac12
gcaggctaaccggaacctgtattatttagtttatgctacgttaaataaagacctttcgttcacataactgaatgtgtaatggccttgagatttcaagcataccaagttggtggagacggggtcgttacaaaagactctttaaacagattctgcctctgaaagcttttggacatgatcagcatcgctctttagaagctcttgctctttcaaattttgagcatttgcaactctaacgtcattttgttggaccaaagttgccctggcttgagccaagaatgcttgatcaacggatgcctttcttgggtttggagcttcaaagacagcttctaattcttctaagcttctacccttagtttcaacgaagaagaagtagataacaataaattcgaaaatatcgaagaaaacgtagaacacatagaaccaatatttgatattcttcattgcctttggagtagcaaattgattaacaaattgggcaacaccagaaaccacaccgttgaggagttgggccttagatctcgtcaagtttgtagacacttctgttgagtacatggattgcattggagtgaaagcaaaagaaaagataacaccaaagagataaatgaacaccaatgcaccattggaagcactcttcttcttagtcttctcataacgagcagtacagatagatagacctgtcaatgctaatgcagcacctgagatagaaccaaggaaaccttcccttctaccaatcttatcaataaagaatgcaccgcaaattgaagaaatccaagagacgatggaataaacaccattcattaacacattcaatgagacactcttcataccaacatttctcaacatggtaggcaaatagtacgaacacacattgttaccggaaaattgaccgaaccaagccataagtataaccaacattgctctgtacctatccgatctcgttctgaataagatccttacatctaacatttctagagggtttgataaatctgtaccatggaaagattctattatttctgccatctccatatccaataatggatgagttctatcgccatttaagtggtatttgataatgaattcacgagcttcttcctcacggccaacaccaaccaaccatcttggagattctgggattaaccaaccaaatatacacacaagacctgggaacatcatttgtaagtataatggaatcttaaaagccttggaggagttagggaagtttttgttggtaccgagagtggtaaaggcagcaacaatggaaccgacagaccaaagggtgttataaagacctgcaaccttacctcttaagtgagctggagccacttctgcacagtatgctggagctgctgaattagcgattgtagcgaaaaaggccatgaaccatctaccaccaattaatgcactctttgttgttgttacagacgtaataataccaccaataacaacacccagacacccaattaaaatagcaggttttctacctttccaatccataagaggaacaaagaatgcaccgcaaatttgaccaacgttgaaaatagagaacactagaccagtaccactggatgagttaatatccaaatggtagtattccaaatatgcattttcggtatagatagaacccattaaagccccatcataaccttgcatagtagcacacagatatgttacaaaacataaactgtacaatttgtaatattgcttcgacaagtaacctggtaagagcacttcctctctagcgtcctcgatggggacaccattgattttcaatccagaagtattatcattatcactgttcaaggcttccttgtgatccagatcaatgcccaaagtgtctttatgctcgatagtattaattggcttcttctgcagcgaagatgagctgctcgaatgatctgccatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaacatgaatgtacggaggtagtttgctgattggcggtctatagataccttggttatggcgccctcacagccggcaggggaagcgcctacgcttgacatctactatatgtaagtatacggccccatatatatatatatatatatacattaaacattattggtaaatacaccagcaaccgcatgattgatgttatggtggaatataggtagctaaaaaaactctacataacaaagtaattgtttacattgatcttgacctatcaataatgatccttccgcaggttcacgttacatgcgtacacccgtcaccggcatgcgatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaatacggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggactactggcaggatcaaccagata

SEQ ID NO: 103 - DNA, 5474 bp, Artificial sequence
  22wcaG_33gmd_54FT
tcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgccaggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaaaaaaatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaaacagcggccgcccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgggtgtacgcatgtaacattatactgaaaaccttgcttgagaaggttttgggacgctcgaaggctttaatttgcggcgcgggtgccggtagaggtgtggtcaataagagcgacctcatactatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgttatgactccagcgcgatcgccacgtcgtagccgtgagatttcagcagagagtgttttttcgccgcttcgaggtcattagccaccatttcagacaccatctctctgagggtgatttccggtttccagcccagtttttcgtgcgctttggtcgggtcgccgagcagcgtttcaacttcagccggacggaagtaacgcgggtcaacagcgataatcacatcacccggtttaacgcccggcgcgtcatgcccggtgacggaaaccacaatgcccttctcttcaacgcccgtgccttcaaagcgcagtttgatgcccagctgtgctgccgccatttccacgaactgacgcacggagtactgaacgccggtcgcgataacgaaatcttccggctgttcctgctgcagcatcatccactgcatttttacgtagtctttggcgtggccccagtcacgcagggaatccatattgccgaggtacaggcacgactccagcccctgggcgatgttggcgattgcgcgggtgattttgcgggtaacgaaggtttcgccgcggcgcggggattcatggttgaagagaattccgttacaggcgtacatgccgtaggattcacggtagttaacggtgatccagtaggcgtacagtttggcgaccgcatacggagatcgcgggtagaacggcgtggtctctttctgcggaatttcctgcaccagaccatacagttcagaggtggaagcctgatagaaacgagttttcttttccagaccgaggaagcggatcgcctccagcaggcgcagcgtacccatcgcgtcgacgtcagcggtatattctggtgactcaaaagagaccgcaacgtggctcattgcgcccaggttgtacacttcatccggctgtacttcacgcaaaatgcgcgtcaggttagaggtatcactcaggtcgccataatgcagatggaatttcgggttgcaggtgtgcggatcctgataaatgtgatccacgcgctcggtgttgaatgacgatgcgcgacgcttaataccatgcacctcgtaacctttttccagcagaaactctgccaggtaagaaccgtcttgtccggttacaccggtgatgagagcgacttttgacattttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctataggttttcagccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataatggcctttaaagttgttcagatttgtggtggtctgggcaatcagatgtttcagtatgcatttgcaaaaagcctgcagaaacatagcaatacaccggttctgctggatattaccagctttgattggagcaatcgtaaaatgcagctggaactgtttccgattgatctgccgtatgcaagcgaaaaagaaattgcaattgccaaaatgcagcatctgccgaaactggttcgtaatgttctgaaatgcatgggttttgatcgtgtgagccaagaaatcgtgtttgaatatgaaccgaaactgctgaaaaccagccgtctgacctatttttatggctattttcaggatccgcgttattttgatgcaattagtccgctgatcaaacagacctttaccctgcctccgcctccggaaaatggtaataacaaaaaaaaagaagaagagtatcatcgtaaactggcactgattctggcagcaaaaaatagcgtgtttgtgcatattcgtcgcggtgattatgttggtattggttgtcagctgggcatcgattatcagaaaaaagcactggaatacatggcaaaacgtgttccgaatatggaactgtttgtgttttgcgaggacctggaatttacccagaatctggatctgggctatccgtttatggatatgaccacccgtgataaagaggaagaggcatattgggatatgctgctgatgcagagctgtaaacatggtattattgccaacagcacctatagttggtgggcagcatatctgattaataacccggaaaaaatcattattggtccgaaacattggctgtttggccatgaaaacatcctgtgtaaagaatgggtgaaaatcgaaagccactttgaagtgaaaagccagaaatataatgcctaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattg

SEQ ID NO: 104 - DNA, 1642 bp, Artificial sequence
  pTDH3_yECit_tENO1
ataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaggttatggtttgatgtgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatcctatcaatctgccttatccaaagatccaaacgaaaagagagatcacatggtcttgttagaatttgttactgctgctggtattacccatggtatggatgaattgtacaaatgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtat

(19) United States
(12) Patent Application Publication
(10) Pub. No.: US 2022/0298530 A1
(43) Pub. Date: Sep. 22, 2022

(54) Optimal Chromosomal Insertion Loci

(71) Applicant: INBIOSE N.V., Zwinjnaarde (BE)

(72) Inventors: Joeri Beauprez, Bredene (BE); Pieter Coussement, Gentbrugge (BE); Sofie De Maeseneire, Destelbergen (BE); Anke Goormans, Gent (BE); Gert Peters, Sint-Amandsberg (BE); Nico Snoeck, Evergem (BE); Dries Van Herpe, Wondelgem (BE)

(73) Assignee: INBIOSE N.V., Zwinjnaarde (BE)

(21) Appl. No.: 17/616,066

(22) PCT Filed: Jun. 4, 2020
(86) PCT No.: PCT/EP2020/065560

§ 371 Date: Dec. 2, 2021

(30) Foreign Application Priority Data
Jun. 4, 2019 (EP) 19178150.9

(51) Int. Cl. C12N 15/90; C12N 15/52

(52) U.S. Cl. CPC C12N 15/905; C12N 15/52

(57) ABSTRACT

The present invention is in the technical field of synthetic biology and metabolic engineering. More particularly, the present invention relates to a method to determine the expression stability of a heterologous gene at a chromosomal location in a cell undergoing burden and to produce mutated cells or organisms transformed with a heterologous gene at a chromosomal location, wherein the expression of said heterologous gene is not influenced by a burden or wherein the expression of said heterologous gene is reduced by a burden. The present invention describes methods to locate interesting chromosomal knock-in locations in a cell. Such engineered cells and organisms are applied for the production of bioproducts, such as but not limited to carbohydrates, lipids, proteins, organic acids, amino acids, alcohols, antibiotics and peptides. Preferably, the invention is applied in the technical field of fermentation of metabolically engineered microorganisms.

The present invention is in the technical field of synthetic biology and metabolic engineering. More particularly, the present invention relates to a method to determine the expression stability of a heterologous gene at a chromosomal location in a cell undergoing burden and to produce mutated cells or organisms transformed with a heterologous gene at a chromosomal location, wherein the expression of said heterologous gene is not influenced by a burden or wherein the expression of said heterologous gene is reduced by a burden. The present invention describes methods to locate interesting chromosomal knock-in locations in a cell. Such engineered cells and organisms are applied for the production of bioproducts, such as but not limited to carbohydrates, lipids, proteins, organic acids, amino acids, alcohols, antibiotics and peptides. Preferably, the invention is applied in the technical field of fermentation of metabolically engineered microorganisms.

### BACKGROUND

The genome of numerous types of cells, for example microorganisms such as Escherichia coli and Saccharomyces cerevisiae, plants such as Arabidopsis thaliana, animals such as Drosophila melanogaster and Danio rerio, were successfully transformed with transgenes in the early 1990's. Over the last thirty years, numerous methodologies have been developed for transforming the genome of cells, like yeast or bacteria, wherein a transgene is stably integrated into the genome of the cell. This evolution of transformation methodologies has resulted in the capability to successfully introduce a transgene coding for a specific enzyme, protein, oil, (oligo)saccharide or other product with commercial interest within the genome of plants, microorganisms and even animals. For example, the introduction of specific genes within microorganisms provided a new and convenient technological innovation for producing a myriad of products in a relatively simple and cost-effective way by fermentation, which was unparalleled in chemical or enzymatic methods.

For example, the microbial host Escherichia coli has been used extensively for the production of metabolites with commercial interest (1-6). Promoter and terminator databases (7-9) are readily available as well as a wide amount of expression vectors (10) and numerous gene editing technologies (11-15). Together with the ever-reducing cost of synthetic DNA, the range of possibilities is expanding even more. Recent advances have secured the possibility of integrating whole synthetic pathways with ease and high efficiency onto the bacterial genome (16, 17), hereby overcoming the need for plasmid expression and their associated instability (18).

In the past, transformation methodologies relied upon the random insertion of transgenes within the genome of the cell. This has several disadvantages. The transgenic events may randomly integrate within gene transcriptional sequences, thereby interrupting the expression of endogenous traits and altering the growth and development of the cell. In addition, the transgenic events may indiscriminately integrate into locations of the genome that are susceptible to gene silencing, culminating in the reduced or complete inhibition of transgene expression either in the first or subsequent generations of transgenic cells. Finally, the random integration of transgenes within the cell's genome requires effort and cost in identifying the location of the transgenic event and selecting transgenic events that perform as designed without any impact to the cell.

Targeted genome modification of a cell is thus the preferred way of working of both applied and basic research. Targeting genes and gene stacks to specific locations in the genome of a cell will improve the quality of transgenic events, reduce costs associated with production of transgenic events and provide new methods for making transgenic products such as sequential gene stacking. Overall, targeting transgenes to specific genomic sites is likely to be commercially beneficial. Methods and compositions have been developed in the recent past to target and cleave genomic DNA by site specific nucleases (e.g., Zinc Finger Nucleases (ZFNs), Meganucleases, Transcription Activator-Like Effector Nucleases (TALENS) and Clustered Regularly Interspaced Short Palindromic Repeats/CRISPR-associated nuclease (CRISPR/Cas) with an engineered crRNA/tracr RNA), to induce targeted mutagenesis, induce targeted deletions of cellular DNA sequences, and facilitate targeted recombination of an exogenous donor DNA polynucleotide within a predetermined genomic locus.

An alternative approach is to target the transgene to preselected target loci within the genome of the cell. In recent years, several technologies have been developed and applied to cells for the targeted delivery of a transgene within the genome of the cell. However, the question of where to incorporate your novel optimized pathway remains unanswered. Historically, non-essential genes and pathogen (viral) integration sites in genomes have been used as loci for targeting. The number of such sites in genomes is rather limiting and there is therefore a need for identification and characterization of targetable optimal genomic loci that can be used for targeting of donor polynucleotide sequences. In addition to being amenable to targeting, optimal genomic loci are expected to be neutral sites that can support transgene expression and will perform under differing process or stress conditions. For example, the genome of Escherichia coli contains more than 4000 genes or 4.64 Mbp and thus numerous positions for the incorporation of your biosynthetic pathway. Few studies have already noted a difference in expression between several locations around the genome. In general, a gene dosage effect is observed in which a gene is higher expressed when located closer to the origin of replication (oriC) due to the higher copy number for genes closer to oriC during replication (30). This gene copy number can range from one to four for locations close to oriC (31). Often in these studies, a reporter cassette is integrated on different genomic locations. One study indicates a two-to-three-fold improvement for a lacZ reporter (32) whereas others measured a four-to-20-fold enhancement using a fluorescent protein (33-35). In contrast, other research states a 300-fold expression difference of a fluorescent reporter and indicates that only 1.4-fold is attributed to the gene dosage effect (36). A recent study of Scholz (62) describes a high-resolution mapping of the transcriptional propensity in E. coli.

Another challenge in metabolic engineering and synthetic biology is the fact that introducing heterologous genes influences the cellular resources significantly, impacting general expression of genes in the cell. Related hereto, Ceroni (61) developed a method to measure the impact of the expression of a heterologous gene on the expression of another heterologous gene in the cell. By changing the expression level of the heterologous gene, via changing the UTR or promoter, the impact on the expression of the second gene was changed. This change is considered a change in metabolic burden on the cell.

### DESCRIPTION OF THE INVENTION

One embodiment of the present disclosure is directed to a method to determine the expression stability of a chromosomal location in a cell. The method comprises providing an isolated cell to be transformed and chromosomally integrating a marker cassette in said cell at said chromosomal location. A burden is then imposed upon said cell comprising said marker cassette. The expression of the marker is determined, both for the cell with and without said burden. When the burden is not influencing the expression of the marker, a stable chromosomal integration location is found. A sensitive location shows a reduced expression due to said burden. In a preferred embodiment a scoring of the expression stability of said chromosomal location of the cell is done.

Another embodiment provides for a method to determine relative expression stability of a chromosomal position or location in a cell. This chromosomal position provides a tuneable chromosomal transformation or insertion location for production of a desired metabolite. In this method a marker cassette is chromosomally integrated in the isolated cell, preferably a host cell. A burden is imposed on the cell which comprises the marker cassette at said chromosomal position or location. The influence of the imposed burden is measured in comparison with a similar cell i) with the integrated marker but without the burden imposed; ii) without the integrated marker but under the same imposed burden and/or iii) in comparison with a cell of the same organism with another integration location of said marker cassette and under the same burden. The influence of the imposed burden is measured by determining the expression of the marker. As such, a relative expression stability of a chromosomal integration location in the cell is obtained. Preferably the performance of said integration location(s) is scored.

One embodiment of the present disclosure is directed to methods of identifying optimal sites in a cell's genome, including for example the Escherichia coli genome, for the insertion of heterologous or exogenous sequences.

One such method will produce stable expression transformants of a cell. The method will first measure the influence of a burden imposed on an isolated cell which has chromosomally integrated a marker cassette. The influence of that burden on the expression of the marker is then compared to the expression of the marker without said burden. The above steps are then repeated for several chromosomal locations and preferably a scoring of the expression of the marker is done. Based on the results of measurement of the expression stability and/or the scoring of the chromosomal locations, a selection can be done for locations providing a stable expression integration location. Such location can then be used for introduction and expression of a heterologous gene, genetic cassette or set of genes into similar untransformed cells thereby producing cells which will, even under a burden, still produce the heterologous gene, genetic cassette or set of genes at the same expression level as without the burden.

Another method for identifying an optimal site provides a method to produce a burden repressible transformant of a cell. Such method will, in the same way as the previous method, first measure the influence of a burden imposed on an isolated cell which has chromosomally integrated a marker cassette. The influence of that burden on the expression of the marker is then compared to the expression of the marker without said burden. The above steps are then repeated for several chromosomal locations and preferably a scoring of the expression of the marker is done. Based on the results of measurement of the stability and/or the scoring of the chromosomal locations, a selection can be done for locations providing a burden repressible or burden sensitive integration location. Such location can then be used for introducing and expression of a heterologous gene, genetic cassette or set of genes into similar untransformed cells thereby producing cells which will be prone to a burden imposed and which will have a reduced expression of the introduced heterologous gene, genetic cassette or set of genes in comparison to expression without burden.

In a further embodiment, a combination of both methods to identify optimal sites can be used to make transgenic cells which have an integrated bioproduction pathway of which the different parts are tuned for optimal bioproduct formation. When a specific part of the pathway poses a bottleneck, this gene or set of genes can be integrated at a chromosomal integration location which was determined as a stable and strong chromosomal location, while other parts of the pathway might be better located to a more burden sensitive chromosomal location.

In still another embodiment, a method is provided for the production of a bioproduct using a genetically modified host cell. The method provides a host cell, which has been genetically modified, such that at least said cell is able to produce the bioproduct, wherein the unmodified host cell is not able to produce the bioproduct, due to the introduction of at least one heterologous gene, encoding the bioproduct or an intermediate thereof, which is expressed in the host cell. That genetically modified host cell is then cultivated and/or grown in a cultivation medium enabling to production of the bioproduct thereby producing the bioproduct obtainable from the medium the host cell is cultivated in. The genetically modified host cell is modified such that the heterologous gene is introduced at a chromosomal location obtainable or obtained from any of the methods described herein. Preferably, the bioproduct as obtained by this method or any of the methods as described herein, is an oligosaccharide as described herein, more preferably sialic acid, a sialylated, fucosylated, or galactosylated oligosaccharide, even more preferably a human milk oligosaccharide as described herein.

Here we also show that it is possible to minimize the effect of heterologous gene expression or suboptimal environmental conditions on other heterologous genes or pathways, or to use the effect of said heterologous genes and/or suboptimal environmental conditions on the expression of heterologous pathway genes.

Applicants have thus constructed a method for identifying locations of native genomic sequences of a cell that are optimal sites for site directed targeted insertion of a heterologous gene.

More particularly, in accordance with one embodiment, applicants have discovered a method to identify genetic loci which are not metabolically influenced by a burden put on the cell, such as e.g. the expression of a plasmid introduced in the cell. As disclosed herein, applicants have discovered a number of loci in the coli genome that meet this criterium and thus represent optimal sites for the insertion of heterologous or exogenous sequences.

In the methods described herein the marker cassette is integrated at any location in the chromosome, but preferably at intergenic region or at a non-essential gene chromosomal locus, even more preferably avoiding regulatory leader sequences, regions that contain promoters, 5′-UTRs, 3′-UTRs, transcription terminators, sigma factors, enhancers or silencers.

The marker cassette is preferably flanked with insulating DNA sequences, wherein said insulating DNA sequences are preferably transcription terminators.

The marker cassette used in any of the methods described herein can by any available marker system for measuring and/or detecting expression, such as, but not limited to any gene or gene product that is used as a reference in molecular biology or a gene of interest that can be measured to score the expression of said marker. Examples of markers are antibiotic resistance genes, auxotrophy complementation genes, fluorescent genes, colorant genes, colorant pathway genes, such as but not limited to carotenoid pathway, violacein pathway, color producing flavonoid pathways, color producing isoprenoid pathways, or any other non-color producing pathway.

Methods to measure the marker expression are commonly known methods in the art such as but not limited to proteome analysis, ELISA, gel electrophoresis analysis, MALDI analysis, mass spectrometry analysis, transcriptome analysis, RTqPCR analysis, micro-array analysis, RNAseq analysis, Riboseq analysis, sequencing, next gen sequencing, and/or nanopore sequencing. In a preferred embodiment, the marker cassette is a fluorescent cassette.

In the methods described herein the imposed burden or metabolic burden can be any burden possible, such as but not limited to a chemical, physical or genetic/expression burden put on the cell so that the cell undergoes a physiological stress that redirects resources such as DNA polymerases, RNA polymerases, ribosomes, protein chaperones, and/or sRNA, to cope with such burden. Non limited chemical burdens are for example high concentrations of medium components, such as but not limited to carbon sources (such as but not limited to glucose, sucrose, glycerol, maltose, amylose, trehalose, galactose, lactose, fucose, sialic acid, n-acetylglucosamine), medium salts (such as but not limited to phosphates, sulfates, nitrates, chlorides, calcium salts, sodium salts, potassium salts, iron salts, magnesium salts, manganese salts, copper salts, zinc salts, cobalt salts, molybdenum salts), complex media (such as but not limited to yeast extract, peptone, casein, casamino acid, whey, wood hydrolysates, lignocellulosic hydrolysates), solvents, acids, amino acids, gene inducers, and/or product precursors. Non limiting physical burdens are for example pH conditions that are non-natural to the cell (for instance a pH offset of equal to or higher than 0.5 compared to the optimal growth pH of said cell), shear stress condition caused by such as but not limited to mixing, pumping, and/or recycling, temperature conditions that are not natural to the cell (for instance a temperature offset of equal to or higher than 1° C. compared to the optimal growth temperature of said cell), pressure conditions that are not natural to the cell (for instance a pressure offset of equal to or higher than 100 mbar compared to the optimal growth pressure of said cell), and/or osmotic pressure that are not natural to the cell. Further examples of a physical burden put on a cell or an organism are: a heat stress, a cold stress, a pest stress, a viral burden, a drought stress, low oxygen, high nitrogen, high UV. Non limiting genetic/expression burdens are for instance the high expression and/or production of protein, peptide, RNA or bioproduct by means of the use of genetic constructs with a strong promoter, UTR, transcription terminator, by means of multiple gene copies, plasmids, by means of the introduction of genetic pathways. In a preferred embodiment of the present invention the burden imposed is the expression of a plasmid.

In the methods described herein a tuneable transformation can be a stable transformation. In other methods described herein a tuneable transformation provides for a relative repression of the integrated marker or heterologous gene under burden, which means that a heterologous gene is integrated at a chromosomal location which is sensitive to burden. As such, when the cell is under a burden, the heterologous gene will have a reduced or stopped expression which is defined herein as a tuned or tuneable transformation of the cell comprising the heterologous gene.

In the methods described herein the cell can be a cell of any organism, and preferably an isolated cell. The term ‘organism’ or ‘cell’ as used herein refers to a microorganism chosen from the list consisting of a bacterium, a yeast or a fungus, or, refers to a plant cell, animal cell, a mammalian cell, an insect cell and a protozoal cell. The latter bacterium preferably belongs to the phylum of the Proteobacteria or the phylum of the Firmicutes or the phylum of the Cyanobactria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the present invention specifically relates to a mutated and/or transformed Escherichia coli strain as indicated above wherein said E. coli strain is a K12 strain. More specifically, the present invention relates to a mutated and/or transformed Escherichia coli strain as indicated above wherein said K12 strain is E. coli MG1655. The latter bacterium belonging to the phylum Firmicutes belongs preferably to the Bacilli, preferably from the species Bacillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Pichia, Hansunella, Kluyveromyces, Yarrowia, Eremothecium, Zygosaccharomyces or Debaromyces. The latter fungus belongs preferably to the genus Rhizopus, Dictyostelium or Aspergillus. “Plant cells” includes cells of flowering and non-flowering plants, as well as algal cells, for example Chlamydomonas, Chlorella, etc. Preferably, said plant cell is a tobacco, alfalfa, rice, tomato, corn, maize or soybean cell; said mammalian cell is a CHO cell or a HEK cell; said insect cell is an S. frugiperda cell and said protozoal cell is a L. tarentolae cell.

In a preferred embodiment the cell is a cell of a microorganism, wherein more preferably said microorganism is a bacterium or a yeast.

In still another embodiment, the present invention provides a method to produce stable transformants of E. coli producing a desired gene, genetic cassette and/or set of genes. The E. coli cells are transformed by the introduction of a desired heterologous gene, genetic cassette and/or set of genes at at least one intergenic position chosen from the list of E. coli genomic intergenic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ.

A further embodiment provides for a method to produce burden repressible transformants of E. coli expressing a desired heterologous gene, genetic cassette and/or set of genes wherein the E. coli cells are transformed by the introduction of a desired heterologous gene, genetic cassette and/or set of genes at at least one intergenic position chosen from the list of E. coli genomic intergenic locations djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

In one embodiment a method is provided to produce a desired bioproduct or metabolite by E.coli, wherein the method comprises providing E. coli cells and providing a bioproduct or metabolite production heterologous gene, genetic cassette and/or set of genes. The coli cells are transformed by the introduction of the desired heterologous gene, genetic cassette and/or set of genes at at least one intergenic positions chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ. Those cells are then grown in a medium permissive for the production of the desired metabolite and/or bioproduct.

In another embodiment a desired bioproduct or metabolite is produced by E.coli, wherein the E. coli cells are transformed with a bioproduct or metabolite production heterologous gene, genetic cassette and/or set of genes at at least one intergenic positions chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ, and/or at at least one intergenic positions chosen from the list of E. coli genomic locations djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

The obtained cells are then grown in a medium permissive for the production of the desired metabolite or bioproduct.

Another aspect of the present invention provides for E. coli chromosome positions to be used for tuneable transformation at at least one intergenic position or location chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ, and/or at at least one intergenic positions chosen from the list of E. coli genomic locations djlA_yabP, frwA_irwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

Preferably, the present invention provides for use of E. coli chromosome position for tuneable transformation by introduction of at least one desired heterologous gene at at least one intergenic chromosome location, wherein said at least one intergenic chromosome location is chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ, and/or at at least one intergenic positions chosen from the list of E. coli genomic locations djlA_yabP, frwA_irwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

More preferably, the present invention provides for use of E. coli chromosome position for tuneable transformation by introduction of at least one desired heterologous gene providing for oligosaccharide synthesis by the cell, at at least one intergenic chromosome location, wherein said at least one intergenic chromosome location is chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ, and/or at at least one intergenic positions chosen from the list of E. coli genomic locations djlA_yabP, frwA_irwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

Still another aspect of the present invention provides an E. coli cell transformed by introduction of a heterologous gene, genetic cassette or set of genes at at least one intergenic location chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ, and/or at at least one intergenic positions chosen from the list of E. coli genomic locations djlA_yabP, frwA_irwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

In a preferred embodiment, the E. coli cell is transformed to produce an oligosaccharide with heterologous genes. The cell is transformed by introduction of a heterologous gene, genetic cassette or set of genes at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

Preferably the oligosaccharide as described herein contains monosaccharides selected from the group comprising Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-Iyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5, 7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5, 7-Diamino-3,5, 7, 9-tetradeoxy-D-glycero-D-galacto-non-2-ulopyranosonic acid, 5, 7-Diamino-3,5, 7, 9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

In one embodiment an E. coli cell is transformed with at least one heterologous gene to produce a sialic acid pathway or sialylation pathway, or fucosylation pathway or galactosylation pathway or N-acetylglucosamine carbohydrate pathway. This cell is transformed by introduction of a heterologous gene, genetic cassette or set of genes at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

A further embodiment of the present invention provides a method to produce a fucosylated, sialylated, galactosylated oligosaccharide or sialic acid with a cell as described herein, respectively.

In a further embodiment, the present invention provides for an E. coli cell transformed to produce a human milk oligosaccharide pathway. In this embodiment, the cell is transformed by introduction of a heterologous gene, genetic cassette or set of genes at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

One embodiment then provides a method to produce a human milk oligosaccharide with the cell described herein. Another embodiment provides a method for the production of a bioproduct using a genetically modified host cell as described herein.

Further embodiments provide for the use of a host cell for the production of a bioproduct wherein said host cell expresses a heterologous protein which heterologous protein's coding sequence was introduced at a location of said host cell, said location being defined or identified by any one of the methods described herein.

### Definitions

The terms bioproduct and metabolite as used herein is any product that can be synthesized in a biological manner, i.e. via enzymatic conversion, microbial biosynthesis, cellular biosynthesis.

Examples of bioproducts and metabolites are:
1) Small organic molecules, such as but not limited to organic acids, alcohols, amino acids; proteins, such as but not limited to enzymes, antibodies, single cell protein, nutritional proteins, albumines, lactoferrin, glycolipids and glycopeptides; antibiotics, such as but not limited to antimicrobial peptides, polyketides , penicillins, cephalosporins, polymyxins, rifamycins, lipiarmycins, quinolones, sulfonamides, macrolides, lincosamides, tetracyclines, aminoglycosides cyclic lipopeptides (such as daptomycin), glycylcyclines (such as tigecycline), oxazolidinones (such as linezolid), lipiarmycins fidaxomicin; lipids, such as but not limited to arachidonic acid, docosahexaenic acid, linoleic acid, Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Tetracosapentaenoic acid Tetracosahexaenoic acid (Nisinic acid); Flavanoids, glycolipids, ceramides, sphingolipids, carbohydrates, monosaccharides, disaccharides, polysaccharides, oligosaccharides such as but not limited to human milk oligosaccharides, glycosaminoglycans, chitosans, chondrotoines, heparosans, Glucuronylated oligosaccharides;
2) A human milk oligosaccharide, such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialylactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides;
3) A ‘sialylated oligosaccharide’, a charged sialic acid containing oligosaccharide, i.e. an oligosaccharide having a sialic acid residue. It has an acidic nature. Some examples are 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Aca-2,3Gal beta -1,3GalNac beta -1,3Gala-1,4Gal beta -1,4Gal), sialylated tetrasaccharide (Neu5Aca-2,3Gal beta -1,4GlcNac beta -14GlcNAc), pentasaccharide LSTD (Neu5Aca-2,3Gal beta -1,4GlcNac beta -1,3Gal beta -1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s), including but not limited to: oligosaccharide moieties of the gangliosides selected from GM3 (3′sialyllactose, Neu5Aca-2,3Gal β-4Glc) and oligosaccharides comprising the GM3 motif, GD3 Neu5Aca-2,8Neu5Aca-2,3Gal β-1,4Glc GT3 (Neu5Aca-2,8Neu5Aca-2,8Neu5Aca-2,3Gal β-1,4Glc); GM2 GaINAc β-1,4(Neu5Aca-2,3)Gal β-1,4Glc, GM1 Gal β-1,3GaINAc β-1,4(Neu5Aca-2,3)Gal β-1,4Glc, GD1a Neu5Aca-2,3Gal β-1,3GaINAc β-1,4(Neu5Aca-2,3)Gal β-1,4Glc GT1a Neu5Aca-2,8Neu5Aca-2,3Gal β-1,3GaINAc β-1,4(Neu5Aca-2,3)Gal β-1,4Glc GD2 GaINAc β-1,4(Neu5Aca-2,8Neu5Aca2,3)Gal β-1,4Glc GT2 GspaINAc β-1,4(Neu5Aca-2,8Neu5Aca-2,8Neu5Aca2,3)Gal β-1,4Glc GD1b, Gal β-1,3GaINAc β-1,4(Neu5Aca-2,8Neu5Aca2,3)Gal β-1,4Glc GT1b Neu5Aca-2,3Gal β-1,3GaINAc β-1,4(Neu5Aca-2,8Neu5Aca2,3)Gal β-1,4Glc GQ1b Neu5Aca-2,8Neu5Aca-2,3Gal β-1,3GaINAc β-1,4(Neu5Aca-2,8Neu5Aca2,3)Gal β-1,4Glc GT1c Gal β-1,3GaINAc β-1,4(Neu5Aca-2,8Neu5Aca-2,8Neu5Aca2,3)Gal β-1,4GIc GQ1c, Neu5Aca-2,3Gal β-1,3GaINAc β-1,4(Neu5Aca-2,8Neu5Aca-2,8Neu5Aca2,3)Gal β-1,4Glc GP1c Neu5Aca-2,8Neu5Aca-2,3Gal β-1,3GaINAc β-1,4(Neu5Aca-2,8Neu5Aca-2,8Neu5Aca2,3)Gal β-1,4Glc GD1a Neu5Aca-2,3Gal β-1,3(Neu5Aca-2,6)GaINAc β-1,4Gal β-1,4Glc Fucosyl-GM1 Fuca-1,2Gal β-1,3GaINAc β-1,4(Neu5Aca-2,3)Gal β-1,4Glc; all of which may be extended to the production of the corresponding gangliosides by reacting the above oligosaccharide moieties with ceramide or synthetizing the above oligosaccharides on a ceramide;
4) A ‘fucosylated oligosaccharide’, generally understood in the state of the art as an oligosaccharide that is carrying a fucose-residue. Examples comprise 2′-fucosyllactose, 3-fucosyllactose, difucosyllactose, lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose;
5) A ‘neutral oligosaccharide’, generally understood in the state of the art as an oligosaccharide that has no negative charge originating from a carboxylic acid group. Examples of such neutral oligosaccharide are 2′-fucosyllactose, 3-fucosyllactose, 2′, 3- difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′- galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose;
6) A monosaccharide as defined herein.

The term polyol as used herein is an alcohol containing multiple hydroxyl groups. For example glycerol, sorbitol, or mannitol.

The term “sialic acid” as used herein refers to the group comprising sialic acid, neuraminic acid, N-acetylneuraminic acid and N-Glycolylneuraminic acid.

Chromosomal loci of essential genes are loci on the chromosome wherein an essential gene is coded. Said essential gene leads to a lethal phenotype when grown in any type of growth condition. Certain genetic deletion of genes lead to conditional growth, such as but not limited to auxotrophic growth, temperature, pH dependent growth. Said genes that lead to such conditional growth are considered to be non-essential genes similar to the genes that do not lead to conditional growth and do not lead to lethal phenotypes.

The terms “transformed to produce an oligosaccharide” as used herein refers to a biochemical pathway consisting of enzymes and their respective genes which lead to the production of a oligosaccharide, such as e.g. a human milk oligosaccharide.

The terms “transformed to produce a human milk oligosaccharide pathway” as used herein refers to a biochemical pathway consisting of enzymes and their respective genes which lead to the production of a human milk oligosaccharide. Such pathways are known in the art and are described in e.g. WO 2012/007481, WO 2013/087884, WO 2016/075243, WO 2018/122225, WO 2012/112777, WO 2015/032412, WO2 019/025485, WO 2018/194411, US 2007020736, WO 2017/188684, WO 2017/042382 and WO 2014/153253.

A ‘fucosylation pathway’ as used herein is a biochemical pathway consisting of the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to alfa 1,2; alfa 1,3 alfa 1,4 or alfa 1,6 fucosylated oligosaccharides or fucosylated oligosaccharide containing bioproduct.

A ‘sialylation pathway’ is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosam ine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetyl mannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to alfa 2,3; alfa 2,6 alfa2,8 sialylated oligosaccharides or sialylated oligosaccharide containing bioproduct.

A ‘galactosylation pathway’ as used herein is a biochemical pathway consisting of the enzymes and their respective genes, galactose-1-epimerase, galactokinase, glucokinase, galactose-1-phosphate uridylyltransferase, UDP-glucose 4-epimerase, glucose--phosphate uridylyltransferase, and/or glucophosphomutase, combined with a galactosyltransferase leading to a alfa or beta bound galactose on the 2, 3, 4, 6 hydroxyl group of a mono, di, oligo or polysaccharide containing bioproduct.

An ‘N-acetylglucosamine carbohydrate pathway’ as used herein is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridylyltransferase, glucosamine-1-phosphate acetyltransferase, glucosamine-1-phosphate acetyltransferase, combined with a galactosyltransferase leading to a alfa or beta bound N-acetylglucosamine on the 3, 4, 6 hydroxylgroup of a mono, di, oligo or polysaccharide containing bioproduct.

The term “recombinant” or “transgenic” or “genetically modified”, as used herein with reference to a cell or host cell indicates that the bacterial cell replicates a heterologous nucleic acid, or expresses a peptide or protein encoded by a heterologous nucleic acid (i.e., a sequence “foreign to said cell” or a sequence “foreign to said location or environment in said cell”). Such cells are described to be transformed with at least one heterologous or exogenous gene, or are described to be transformed by the introduction of at least one heterologous or exogenous gene. Recombinant or transgenic cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, such as replacement of a promoter; site-specific mutation; and related techniques. Accordingly, a “recombinant polypeptide” is one which has been produced by a recombinant cell. A “heterologous sequence” or a “heterologous nucleic acid”, as used herein, is one that originates from a source foreign to the particular cell (e.g. from a different species), or, if from the same source, is modified from its original form. Thus, a heterologous nucleic acid operably linked to a promoter is from a source different from that from which the promoter was derived, or, if from the same source, is modified from its original form. The heterologous sequence may be stably introduced, e.g. by transfection, transformation, conjugation or transduction, into the genome of the host microorganism cell, wherein techniques may be applied which will depend on the host cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989).

Moreover, the present invention relates to the following specific embodiments:

1. Method to determine the expression stability of a chromosomal location in a cell, said method comprising:
providing a cell to be transformed;
chromosomally integrating a marker cassette in said cell at said chromosomal location;
imposing a burden upon said cell comprising said marker cassette;
determining the expression of the marker with and without said burden, wherein i) a stable location is not influenced by said burden or ii) a sensitive location shows a reduced expression due to said burden;
preferably scoring said expression stability of said chromosomal location of said cell, preferably said cell is an isolated cell.

2. Method to determine relative expression stability of a chromosomal position in a cell, said chromosomal position providing a tuneable transformation location for production of a desired metabolite, said method comprising the following steps:
providing a cell;
chromosomally integrating in said cell a marker cassette;
imposing a burden upon said cell comprising said marker cassette at said chromosomal position;
measuring the influence of the imposed burden in comparison with said cell i) with the integrated marker but without the burden imposed; ii) without the integrated marker but under the same imposed burden and/or iii) in comparison with a cell of the same organism with another integration location of said marker cassette and under the same burden;
preferably scoring the performance of said integration location(s).

3. Method to produce stable expression transformants of a cell, said method comprising:
a) i) providing a cell;
   ii) chromosomally integrating in said cell a marker cassette;
   iii) imposing a burden upon said cell comprising said marker;
   iv) measuring the influence of the imposed burden in comparison with said cell without said burden;
   v) repeating steps a) i) to iv) for several chromosomal integration locations;
   vi) selecting the cells with a good or unchanged production of the marker under burden thereby obtaining or identifying the desired location(s);
b) providing untransformed cells
   transforming said untransformed cells with a desired gene, genetic cassette or set of genes at the location obtained from step a) vi).

4. Method to produce a burden repressible transformant of a cell, said method comprising:
a) i) providing a cell;
   ii) chromosomally integrating in said cell a marker cassette;
   iii) imposing a burden upon said cell comprising said marker;
   iv) measuring the influence of the imposed burden in comparison with said cell without said burden;
   v) repeating steps a) i) to iv) for several chromosomal integration locations;
   vi) selecting the cells with a reduced production of the marker under burden thereby obtaining or identifying the desired burden repressible location(s);
b) providing untransformed cells
   transforming said untransformed cells with a desired heterologous gene, genetic cassette or set of genes at said location obtained from step a) vi).

5. Method for the production of a bioproduct using a genetically modified host cell, the method comprising the steps of:
providing a host cell, which has been genetically modified, such, that at least said cell is able to produce the bioproduct wherein the unmodified host cell is not able to produce the bioproduct, due to the introduction of at least one heterologous gene, encoding the bioproduct or an intermediate thereof, which is expressed in the host cell;
cultivating and/or growing said genetically modified host cell in a cultivation medium enabling to production of the bioproduct thereby producing the bioproduct obtainable from the medium the host cell is cultivated in;
characterised in that the heterologous gene is introduced at a chromosomal location obtainable from the method of any one of embodiments 1 to 4.

6. Method according to any one of embodiments 1 to 5, wherein said marker cassette is integrated at a non-essential gene chromosomal locus or at an intergenic region, preferably avoiding regulatory leader sequences, regions that contain promoters, 5′-UTRs, 3′-UTRs, transcription terminators, sigma factors, enhancers or silencers.

7. The method according to any one of embodiments 1 to 6 wherein the marker cassette is flanked with insulating DNA sequences, wherein said insulating DNA sequences are preferably transcription terminators.

8. The method according to any one of embodiments 1 to 7 wherein the marker cassette is an antibiotic resistance cassette, a colorant cassette or a fluorescent cassette.

9. The method according to any one of embodiments 1 to 8 wherein the imposed burden is a chemical, physical or genetic/expression burden, preferably the genetic/expression burden is the expression of a plasmid, preferably a chemical burden is a high concentration of at least one medium component, preferably a physical burden is a non-natural pH, a shear stress condition, a non-natural temperature or cold or heat stress, non-natural pressure conditions, and/or osmotic pressure.

10. The method according to any one of embodiments 2 and 5 to 9, wherein the tuneable transformation is a stable transformation.

11. The method according to any one of embodiments 2 and 5 to 9, wherein the tuneable transformation is a relative repression of the integrated marker or heterologous gene under burden.

12. The method according to any one of embodiments 1 to 11 wherein the cell is a cell of a microorganism, plant, or animal, preferably said microorganism is a bacterium, fungus or a yeast, preferably said plant is a rice, cotton, rapeseed, soy, maize or corn plant, preferably said animal is an insect, fish, bird or mammal.

13. Method to produce stable transformants of E. coli expressing a desired gene, genetic cassette and/or set of genes, said method comprising the following steps:
providing E. coli cells,
transforming said cells by the introduction of a desired heterologous gene, genetic cassette or set of genes at at least one intergenic position chosen from the list of E. coli genomic intergenic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ.

14. Method to produce burden repressible transformants of E. coli expressing a desired heterologous gene, genetic cassette and/or set of genes comprising the following steps:
providing E. coli cells,
transforming said cells by the introduction of a desired heterologous gene, genetic cassette and/or set of genes at at least one intergenic position chosen from the list of E. coli genomic intergenic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

15. Method to produce a desired bioproduct or metabolite by E.coli, said method comprising the following steps:
providing E. coli cells,
providing a bioproduct or metabolite production heterologous gene, genetic cassette and/or set of genes
transforming said cells by introduction of said desired heterologous gene, genetic cassette or set of genes at at least one intergenic positions chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ
growing said cells in a medium permissive for the production of the desired bioproduct or metabolite.

16. Method to produce a desired bioproduct or metabolite by E.coli, said method comprising the following steps:
providing E. coli cells,
providing a bioproduct or metabolite production heterologous gene, genetic cassette and/or set of genes
transforming said cells with said desired heterologous gene, genetic cassette and/or set of genes at at least one intergenic positions chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT;
growing said cells in a medium permissive for the production of the desired bioproduct or metabolite.

17. E. coli chromosome positions to be used for tuneable transformation by introduction of at least one desired heterologous gene at at least one intergenic position chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

18. An E. coli cell transformed by the introduction of at least one heterologous gene at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic location chosen from the list of E. coli genomic locations djlA_yabP, frwA_irwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

19. An E. coli cell transformed by the introduction of heterologous genes to produce an oligosaccharide, said cell transformed with at least one gene, genetic cassette or set of genes at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

20. An E. coli cell according to embodiment 19, wherein said oligosaccharide contains monosaccharides selected from the group comprising: glucose, galactose, N-acetylglucosamine, glucosamine, mannose, xylose, N-acetylmannosamine, N-acetylneureminic acid, N-glycolylneuraminic acid, a sialic acid, N-acetylgalactosamine, galactosamine, fucose, rhamnose, glucuronic acid, gluconic acid, fructose, polyols.

21. An E. coli cell transformed by the introduction of at least one heterologous gene to produce a sialic acid pathway, sialylation pathway, or fucosylation pathway or galactosylation pathway or N-acetylglucosamine carbohydrate pathway said cell transformed at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

22. Method to produce a fucosylated, sialylated, galactosylated oligosaccharide or sialic acid with a cell according to any one of embodiments 19 to 21, respectively.

23. An E. coli cell transformed to produce a human milk oligosaccharide pathway, said cell transformed by the introduction of at least one gene at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ and/or at at least one intergenic positions chosen from the list of E. coli genomic locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

24. Method to produce a human milk oligosaccharide with the cell according to embodiment 23.

25. Method for the production of a bioproduct using a genetically modified host cell according to any one of embodiments 17-21, 23.

26. Use of a host cell for the production of a bioproduct wherein said host cell expresses a heterologous protein which heterologous protein's coding sequence was introduced at a location of said host cell, said location being defined by any one of the methods of embodiments 1 to 12.

In a preferred aspect, the present invention relates to the following preferred specific embodiments:

1. Method to determine the expression stability of a chromosomal location in an isolated cell, said method comprising:
providing an isolated cell to be transformed;
chromosomally integrating a marker cassette in said cell at said chromosomal location;
imposing a burden upon said cell comprising said marker cassette;
determining the expression of the marker with and without said burden, wherein i) a stable location is not influenced by said burden or ii) a sensitive location shows a reduced expression due to said burden;
preferably scoring said expression stability of said chromosomal location of said cell.

2. Method to determine relative expression stability of a chromosomal location in an isolated cell, said chromosomal location providing a tuneable integration location for production of a desired metabolite, said method comprising the following steps:
providing an isolated cell;
chromosomally integrating a marker cassette in said cell at said chromosomal location;
imposing a burden upon said cell comprising said marker cassette at said chromosomal location;
measuring the influence of the imposed burden in comparison with said cell i) with the integrated marker but without the burden imposed; ii) without the integrated marker but under the same imposed burden and/or iii) in comparison with an isolated cell of the same organism with another integration location of said marker cassette and under the same burden, by determining the expression of the marker;
preferably scoring the performance of said integration location(s).

3. Method to produce stable expression transformants of an isolated cell, said method comprising:
a) i) providing an isolated cell;
   ii) chromosomally integrating in said cell a marker cassette;
   iii) imposing a burden upon said cell comprising said marker;
   iv) measuring the influence of the imposed burden in comparison with said cell without said burden;
   v) repeating steps a) i) to iv) for several chromosomal integration locations;
   vi) selecting the cells with a good or unchanged production of the marker under burden thereby obtaining or identifying the desired stable expression location(s);
b) providing untransformed isolated cells
   transforming said untransformed cells with a desired gene, genetic cassette or set of genes at the location obtained from step a) vi).

4. Method to produce a burden repressible transformant of an isolated cell, said method comprising:
a) i) providing an isolated cell;
   ii) chromosomally integrating in said cell a marker cassette;
   iii) imposing a burden upon said cell comprising said marker;
   iv) measuring the influence of the imposed burden in comparison with said cell without said burden;
   v) repeating steps a) i) to iv) for several chromosomal integration locations;
   vi) selecting the cells with a reduced production of the marker under burden thereby obtaining or identifying the desired burden repressible location(s);
b) providing untransformed isolated cells
   transforming said untransformed cells with a desired heterologous gene, genetic cassette or set of genes at said location obtained from step a) vi).

5. Method according to any one of preferred specific embodiment 1 to 4, wherein said marker cassette is integrated at a non-essential gene chromosomal locus or at an intergenic region, preferably avoiding regulatory leader sequences, regions that contain promoters, 5′-UTRs, 3′-UTRs, transcription terminators, sigma factors, enhancers or silencers.

6. The method according to any one of preferred specific embodiment 1 to 5 wherein the marker cassette is flanked with insulating DNA sequences, wherein said insulating DNA sequences are preferably transcription terminators.

7. The method according to any one of preferred specific embodiment 1 to 6 wherein the marker cassette is an antibiotic resistance cassette, a colorant cassette or a fluorescent cassette.

8. The method according to any one of preferred specific embodiment 1 to 7 wherein the imposed burden is a chemical, physical or genetic/expression burden, preferably the genetic/expression burden is the expression of a plasmid, preferably a chemical burden is a high concentration of at least one medium component, preferably a physical burden is a non-natural pH, a shear stress condition, a non-natural temperature or cold or heat stress, non-natural pressure conditions, and/or osmotic pressure.

9. The method according to any one of preferred specific embodiment 2 and 5 to 8, wherein the tuneable transformation is a stable transformation.

10. The method according to any one of preferred specific embodiment 2 and 5 to 8, wherein the tuneable transformation is a relative repression of the integrated marker or heterologous gene under burden.

11. Method for the production of a bioproduct using a genetically modified host cell, the method comprising the steps of:
providing a host cell, which has been genetically modified, such, that at least said cell is able to produce the bioproduct wherein the unmodified host cell is not able to produce the bioproduct, due to the introduction of at least one heterologous gene, encoding the bioproduct or an intermediate thereof, which is expressed in the host cell;
cultivating and/or growing said genetically modified host cell in a cultivation medium enabling to production of the bioproduct thereby producing the bioproduct obtainable from the medium the host cell is cultivated in;
characterised in that the heterologous gene is introduced at a chromosomal location obtainable from the method of any one of preferred specific embodiment 1 to 10.

2. The method according to any one of preferred specific embodiment 1 to 11 wherein the cell is a cell of a microorganism, plant, or animal, preferably said microorganism is a bacterium, fungus or a yeast, preferably said plant is a rice, cotton, rapeseed, soy, maize or corn plant, preferably said animal is an insect, fish, bird or mammal.

13. Method to produce stable transformants of E. coli expressing a desired gene, genetic cassette and/or set of genes, said method comprising the following steps:
providing E. coli cells,
transforming said cells by the introduction of a desired heterologous gene, genetic cassette or set of genes at at least one intergenic position chosen from the list of E. coli genomic intergenic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ.

14. Method to produce burden repressible transformants of E. coli expressing a desired heterologous gene, genetic cassette and/or set of genes comprising the following steps:
providing E. coli cells,
transforming said cells by the introduction of a desired heterologous gene, genetic cassette and/or set of genes at at least one intergenic position chosen from the list of E. coli genomic intergenic locations djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

15. Method to produce a desired bioproduct or metabolite by E.coli, said method comprising the following steps:
providing E. coli cells,
providing a bioproduct or metabolite production heterologous gene, genetic cassette and/or set of genes
transforming said cells by introduction of said desired heterologous gene, genetic cassette or set of genes at at least one intergenic positions chosen from the list of E. coli genomic locations ypjC_ileY, yjip_yjiR, ykgH_betA, thrW_ykfN, ykgA_ykgQ, ileY_ygaQ, ybfC_ybfQ, yeeJ_yeeL, ymgF_ycgH and cspF_quuQ
growing said cells in a medium permissive for the production of the desired bioproduct or metabolite.

16. Method to produce a desired bioproduct or metabolite by E. coli, said method comprising the following steps:
providing E. coli cells,
providing a bioproduct or metabolite production heterologous gene, genetic cassette and/or set of genes
transforming said cells with said desired heterologous gene, genetic cassette and/or set of genes at at least one intergenic positions chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ, djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT;
growing said cells in a medium permissive for the production of the desired bioproduct or metabolite.

17. Method according to any one of preferred specific embodiment 11, 12, 15 or 16, wherein said bioproduct is an oligosaccharide, preferably sialic acid or sialylated, fucosylated, galactosylated oligosaccharide, more preferably a human milk oligosaccharide.

18. Use of E. coli chromosome position for tuneable transformation by introduction of at least one desired heterologous gene at at least one intergenic chromosome location, wherein said at least one intergenic chromosome location is chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH, cspF_quuQ, djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

19. An E. coli cell transformed by the introduction of at least one heterologous gene at at least one intergenic location chosen from the list of E. coli genomic intergenic locations yjip_yjiR, ykgH_betA, thrW_ykfN, ykgA_ykgQ, ileY_ygaQ, ybfC_ybfQ, yeeJ_yeeL, ymgF_ycgH and cspF_quu, djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

20. An E. coli cell transformed by the introduction of heterologous gene to produce an oligosaccharide, said cell transformed with at least one gene, genetic cassette or set of genes at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH, cspF_quuQ djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

21. An E. coli cell according to preferred specific embodiment 20, wherein said oligosaccharide contains monosaccharides selected from the group comprising: glucose, galactose, N-acetylglucosamine, glucosamine, mannose, xylose, N-acetylmannosamine, N-acetylneureminic acid, N-glycolylneuraminic acid, a sialic acid, N-acetylgalactosamine, galactosamine, fucose, rhamnose, glucuronic acid, gluconic acid, fructose, polyols.

22. An E. coli cell transformed by the introduction of at least one heterologous gene to produce a sialic acid pathway, N-acetylglucosamine carbohydrate pathway, sialylation pathway, or fucosylation pathway or galactosylation pathway, said cell transformed at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH cspF_quuQ, djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

23. Method to produce a sialic acid or sialylated, fucosylated, galactosylated oligosaccharide with a cell according to any one of preferred specific embodiment 20 to 22, respectively.

24. An E. coli cell transformed to produce a human milk oligosaccharide pathway, said cell transformed by the introduction of at least one gene at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH, cspF_quuQ, djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

25. Method to produce a human milk oligosaccharide with the cell according to preferred specific embodiment 24.

26. Method for the production of a bioproduct using a genetically modified host cell according to any one of preferred specific embodiment 18 to 22, or 24.

27. Method according to preferred specific embodiment 26, wherein said bioproduct is an oligosaccharide, preferably a human milk oligosaccharide.

28. Use of a host cell for the production of an oligosaccharide wherein said host cell expresses a heterologous protein which heterologous protein's coding sequence was introduced at a location of said host cell, said location being defined by any one of the methods of preferred specific embodiment 1 to 12.

The following drawings and examples will serve as further illustration and clarification of the present invention and are not intended to be limiting.

### BRIEF DESCRIPTION OF THE FIGURES

FIG. 1: Genomic map of Escherichia coli str. K-12 substr. MG1655 with 50 intergenic regions shown as dots, 26 regions indicated with grey dots are discussed more in detail herein. The four macrodomains Right, Ter, Left, and Ori, and the two non-structured regions NS-Right (NS-R) and NS-Left (NS-L) are indicated in grey areas, their borders are according to Espeli et al. (31). The chromosome positions of the terminus (dif; 1,604 kb) and origin of replication (oriC; 3,924 kb) are also labelled. The map was created with CiVi (55).

FIG. 2: Fluorescence of the Dasher reporter cassette corrected for wildtype fluorescence and OD600 (A.U.) and measured at the start of the stationary phase in function of (A) the spread over the genome (kb) and (B) the nett distance from oriC (kb). The linear regression is significant (95%) with an F-statistic of 82.11 and a p-value of 5.76e-12 (see Table 5). Diamonds represent regions within a heEPOD and triangles represent regions within a tsEPOD. The chromosome positions of the terminus (dif; 1,604 kb) and origin of replication (oriC; 3,924 kb) are also labelled. Error bars represent standard deviation of at least 4 replicates.

FIG. 3: Flow cytometry analysis of our 26 strains containing the Dasher sequence on the genome and the burden plasmid pLys-M1. The top barplot shows the fluorescent output of Dasher with (lighter grey “longer” bars) and without (darker grey “shorter” bars) induction of the burden cassette. Strains indicated with an * have a significantly diminished (p<0.05) fluorescent output of the reporter cassette due to the imposed burden. The middle barplot shows the relative fluorescence of Dasher of induction over control. Strains indicated in darker grey, the significant strains from the top barplot, were compared to check if they were equally influenced by the imposed burden, statistical significance is indicated with an *. The bottom barplot shows the fluorescence of the VioB-mCherry cassette with (lighter grey long bars) and without (darker grey short bars) induction. Statistical output can be found in Tables 9 and 10.

FIG. 4: Comparison of fluorescent proteins Dasher, mCherry, and mKate2 on nine locations spread over the genome of E. coli. Fluorescence output is corrected for OD600 measurements and wildtype fluorescence. Error bars represent the standard deviation of 6 replicates.

FIG. 5: Expression strength of tested loci as shown by the fluorescence output of the reporter cassette at the start of stationary phase.

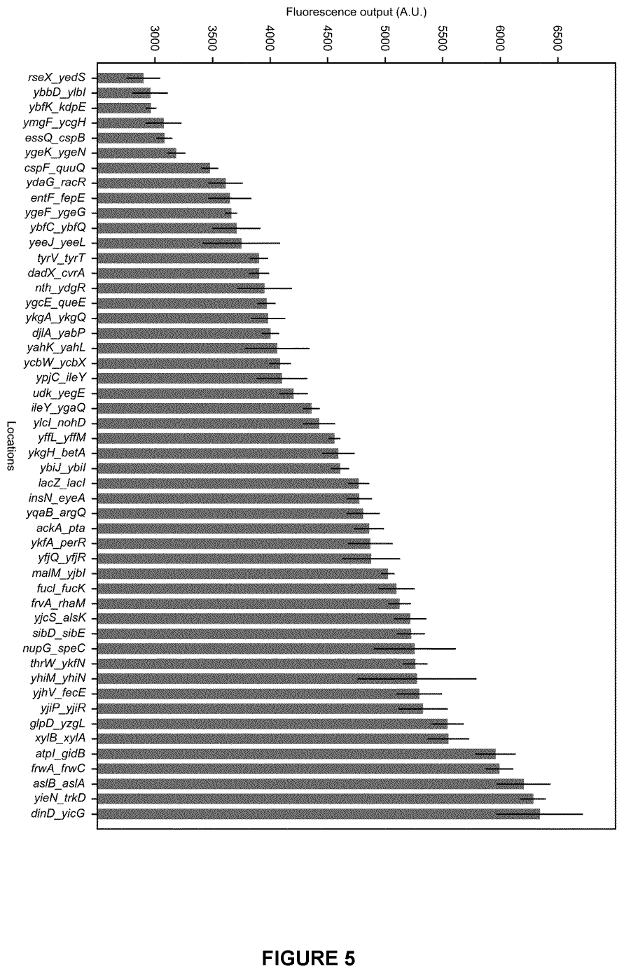

### EXAMPLES SECTION

### Example 1: Materials and Methods

Bacterial Strains and Plasmids

E. coli str. K-12 substr. MG1655 was used for all experiments. The donor plasmids contained a temperature sensitive pSC101 ori, a kanamycin resistance gene and serine integrase attachment (attB) sites flanking the gene of interest with a CC and TT dinucleotide core respectively (37). Different fluorescent proteins were used: sfGFP (38), mKate2 (39), mCherry (40), and several Paintbox proteins (ATUM, USA). Expression is driven by the proD promoter (41) with RBS Bba_B0034 (http://parts.iqem.orq/) and rnpB T1 was chosen as the terminator (42). Donor plasmids were constructed using Golden Gate (43).

The landing pad plasmid pLP consists of the pSC101 ori, a kanamycin resistance gene, and the tetA resistance cassette flanked with attP sites with a CC and TT dinucleotide core respectively (37) (SEQ ID No 1). The vector pInt1 is the same as previously described (17). All constructs were verified by DNA sequencing before use (Macrogen Europe, the Netherlands).

The plasmid pLys-M1 (Addgene plasmid #109382) was a gift from Tom Ellis (44). Bacterial strains and plasmids used in this study are listed in Tables 1 and 2 respectively. The full sequence of the plasmids pLP and pDasher can be found in Tables 3 and 4 respectively.

TABLE 1
Strain list.

| Strain | Description |
|---|---|
| sLOC001 | E. coli K-12 MG1655 |
| SLOC002 | sLOC001 + pLP |
| SLOC003 | sLOC001 + pInt1 (1) |
| SLOC004 | sLOC001 + pDasher |
| SLOC005 | sLOC001 + pmCherry |
| SLOC006 | sLOC001 + pmKate2_02 |
| SLOC007 | sLOC001 + pDasherRV |
| SLOC008 | sLOC001 + pLys-M1 (2) |
| SLOC009 | sLOC001 djlM_yabP::attL_CC-proD-Bba_B0034-Dasher-rnpB_T1-attR_TT |
| SLOC010 | sLOC001 ylcI_nohD::attL_CC-proD-Bba_B0034-Dasher-rnpB_T1-attR_TT |
| SLOC011 | sLOC001 tyrV_fyrT::attL_CC-proD-Bba_B0034-Dasher-rnpB_T1-attR_TT |
| SLOC012 | sLOC001 ypjC_ileY::attL_CC-proD-Bba_B0034-Dasher-rnpB_T1-attR_TT |
| SLOC013 | sLOC001 yhiM_yhiN::attL_CC-proD-Bba_B0034-Dasher-rnpB_T1-attR_TT |
| SLOC014 | sLOC001 thrW_ykfN::attL_CC-proD-Bba_B0034-Dasher-rnpB_T1-attR_TT |
| SLOC015 | sLOC001 entF_fepE::attL_CC-proD-Bba_B0034-Dasher-rnpB_T1-attR_TT |
| SLOC016 | sLOC001 ydaG_racR::attL_CC-proD-Bba_B0034-Dasher-rnpB_T1-attR_TT |
| SLOC017 | sLOC001 ileY_ygaQ::attL_CC-proD-Bba_B0034-Dasher-rnpB_T1-attR_TT |
| SLOC018 | sLOC001 dinD_yicG::attL_CC-proD-Bba_B0034-Dasher-rnpB_T1-attR_TT |
| SLOC019 | sLOC001 ykfA_perR::attL_CC-proD-Bba_B0034-Dasher-rnpB_T1-attR_TT |
| SLOC020 | sLOC001 ybfK_kdpE::attL_CC-proD-Bba_B0034-Dasher-rnpB_T1-attR_TT |
| SLOC021 | sLOC001 cspF_quuQ::attL_CC-proD-Bba_B0034-Dasher-rnpB_T1-attR_TT |
| SLOC022 | sLOC001 yqaB_argQ::attL_CC-proD-Bba_B0034-Dasher-rnpB_T1-attR_TT |
| SLOC023 | sLOC001 frvA_rhaM::attL_CC-proD-Bba_B0034-Dasher-rnpB_T1-attR_TT |
| SLOC024 | sLOC001 insN_eyeA::attL_CC-proD-Bba_B0034-Dasher-rnpB_T1-attR_TT |
| SLOC025 | sLOC001 ybfC_ybfQ::attL_CC-proD-Bba_B0034-Dasher-rnpB_T1-attR_TT |
| SLOC026 | sLOC001 rseX_yedS::attL_CC-proD-Bba_B0034-Dasher-rnpB_T1-attR_TT |
| SLOC027 | sLOC001 ygcE_queE::attL_CC-proD-Bba_B0034-Dasher-rnpB_T1-attR_TT |
| SLOC028 | sLOC001 frwA_frwC::attL_CC-proD-Bba_B0034-Dasher-rnpB_T1-attR_TT |
| SLOC029 | sLOC001 ykgA_ykgQ::attL_CC-proD-Bba_B0034-Dasher-rnpB_T1-attR_TT |
| SLOC030 | sLOC001 ybiJ_ybiI::attL_CC-proD-Bba_B0034-Dasher-rnpB_T1-attR_TT |
| SLOC031 | sLOC001 yeeJ_yeeL::attL_CC-proD-Bba_B0034-Dasher-rnpB_T1-attR_TT |
| SLOC032 | sLOC001 ygeF_ygeG::attL_CC-proD-Bba_B0034-Dasher-rnpB_T1-attR_TT |
| SLOC033 | sLOC001 malM_yjbI::attL_CC-proD-Bba_B0034-Dasher-rnpB_T1-attR_TT |
| SLOC034 | sLOC001 ykgH_betA::attL_CC-proD-Bba_B0034-Dasher-rnpB_T1-attR_TT |
| SLOC035 | sLOC001 ymgF_ycgH::attL_CC-proD-Bba_B0034-Dasher-rnpB_T1-attR_TT |
| SLOC036 | sLOC001 udk_yegE::attL_CC-proD-Bba_B0034-Dasher-rnpB_T1-attR_TT |
| SLOC037 | sLOC001 ygeK_ygeN::attL_CC-proD-Bba_B0034-Dasher-rnpB_T1-attR_TT |
| SLOC038 | sLOC001 yjcS_alsK::attL_CC-proD-Bba_B0034-Dasher-rnpB_T1-attR_TT |
| SLOC039 | sLOC001 yahK_yahL::attL_CC-proD-Bba_B0034-Dasher-rnpB_T1-attR_TT |
| SLOC040 | sLOC001 dadX_cvrA::attL_CC-proD-Bba_B0034-Dasher-rnpB_T1-attR_TT |
| SLOC041 | sLOC001 yffL_yffM::attL_CC-proD-Bba_B0034-Dasher-rnpB_T1-attR_TT |
| SLOC042 | sLOC001 sibD_sibE::attL_CC-proD-Bba_B0034-Dasher-rnpB_T1-attR_TT |
| SLOC043 | sLOC001 yjhV_fecE::attL_CC-proD-Bba_B0034-Dasher-rnpB_T1-attR_TT |
| SLOC044 | sLOC001 yfjQ_yfjR::attL_CC-proD-Bba_B0034-Dasher-rnpB_T1-attR_TT |
| SLOC045 | sLOC001 glpD_yzgL::attL_CC-proD-Bba_B0034-Dasher-rnpB_T1-attR_TT |
| SLOC046 | sLOC001 yjiP_yjiR::attL_CC-proD-Bba_B0034-Dasher-rnpB_T1-attR_TT |
| SLOC047 | sLOC001 lacZ_lacI::attL_CC-proD-Bba_B0034-Dasher-rnpB_T1-attR_TT |
| SLOC048 | sLOC001 ycbW_ycbX::attL_CC-proD-Bba_B0034-Dasher-rnpB_T1-attR_TT |
| SLOC049 | sLOC001 nupG_speC::attL_CC-proD-Bba_B0034-Dasher-rnpB_T1-attR_TT |
| SLOC050 | sLOC001 aslB_aslA::attL_CC-proD-Bba_B0034-Dasher-rnpB_T1-attR_TT |
| SLOC051 | sLOC001 atpI_gidB::attL_CC-proD-Bba_B0034-Dasher-rnpB_T1-attR_TT |
| SLOC052 | sLOC001 yieN_trkD::attL_CC-proD-Bba_B0034-Dasher-rnpB_T1-attR_TT |
| SLOC053 | sLOC001 ybbD_ylbI::attL_CC-proD-Bba_B0034-Dasher-rnpB_T1-attR_TT |
| SLOC054 | sLOC001 essQ_cspB::attL_CC-proD-Bba_B0034-Dasher-rnpB_T1-attR_TT |
| SLOC055 | sLOC001 nth_ydgR::attL_CC-proD-Bba_B0034-Dasher-rnpB_T1-attR_TT |
| SLOC056 | sLOC001 ackA_pta::attL_CC-proD-Bba_B0034-Dasher-rnpB_T1-attR_TT |
| SLOC057 | sLOC001 fucI_fucK::attL_CC-proD-Bba_B0034-Dasher-rnpB_T1-attR_TT |
| SLOC058 | sLOC001 xylB_xylA::attL_CC-proD-Bba_B0034-Dasher-rnpB_T1-attR_TT |
| SLOC059 | sLOC001 dadX_cvrA::attL_CC-proD-Bba_B0034-mCherry-rnpB_T1-attR_TT |
| SLOC060 | sLOC001 sibD_sibE::attL_CC-proD-Bba_B0034-mCherry-rnpB_T1-attR_TT |
| SLOC061 | sLOC001 entF_fepE::attL_CC-proD-Bba_B0034-mCherry-rnpB_T1-attR_TT |
| SLOC062 | sLOC001 ydaG_racR::attL_CC-proD-Bba_B0034-mCherry-rnpB_T1-attR_TT |
| SLOC063 | sLOC001 ykgH_betA::attL_CC-proD-Bba_B0034-mCherry-rnpB_T1-attR_TT |
| SLOC064 | sLOC001 ygeK_ygeN::attL_CC-proD-Bba_B0034-mCherry-rnpB_T1-attR_TT |
| SLOC065 | sLOC001 yjcS_alsK::attL_CC-proD-Bba_B0034-mCherry-rnpB_T1-attR_TT |
| SLOC066 | sLOC001 essQ_cspB::attL_CC-proD-Bba_B0034-mCherry-rnpB_T1-attR_TT |
| SLOC067 | sLOC001 nth_ydgR::attL_CC-proD-Bba_B0034-mCherry-rnpB_T1-attR_TT |
| SLOC068 | sLOC001 dadX_cvrA::attL_CC-proD-Bba_B0034-mKate2_02-rnpB_T1-attR_TT |
| SLOC069 | sLOC001 sibD_sibE::attL_CC-proD-Bba_B0034-mKate2_02-rnpB_T1-attR_TT |
| SLOC070 | sLOC001 entF_fepE::attL_CC-proD-Bba_B0034-mKate2_02-rnpB_T1-attR_TT |
| SLOC071 | sLOC001 ydaG_racR::attL_CC-proD-Bba_B0034-mKate2_02-rnpB_T1-attR_TT |
| SLOC072 | sLOC001 ykgH_betA::attL_CC-proD-Bba_B0034-mKate2_02-rnpB_T1-attR_TT |
| SLOC073 | sLOC001 ygeK_ygeN::attL_CC-proD-Bba_B0034-mKate2_02-rnpB_T1-attR_TT |
| SLOC074 | sLOC001 yjcS_alsK::attL_CC-proD-Bba_B0034-mKate2_02-rnpB_T1-attR_TT |
| SLOC075 | sLOC001 essQ_cspB::attL_CC-proD-Bba_B0034-mKate2_02-rnpB_T1-attR_TT |
| SLOC076 | sLOC001 nth_ydgR::attL_CC-proD-Bba_B0034-mKate2_02-rnpB_T1-attR_TT |
| SLOC077 | sLOC001 djlA_yabP::attR_TT-proD-Bba_B0034-Dasher-rnpB_T1-attL_CC (inverted) |
| SLOC078 | sLOC001 tyrV_tyrT::attR_TT-proD-Bba_B0034-Dasher-rnpB_T1-attL_CC (inverted) |
| SLOC079 | sLOC001 ypjC_ileY::attR_TT-proD-Bba_B0034-Dasher-rnpB_T1-attL_CC (inverted) |
| SLOC080 | sLOC001 yhiM_yhiN::attR_TT-proD-Bba_B0034-Dasher-rnpB_T1-attL_CC (inverted) |
| SLOC081 | sLOC001 thrW_ykfN::attR_TT-proD-Bba_B0034-Dasher-rnpB_T1-attL_CC (inverted) |
| SLOC082 | sLOC001 ileY_ygaQ::attR_TT-proD-Bba_B0034-Dasher-rnpB_T1-attL_CC (inverted) |
| SLOC083 | sLOC001 ybfK_kdpE::attR_TT-proD-Bba_B0034-Dasher-rnpB_T1-attL_CC (inverted) |
| SLOC084 | sLOC001 cspF_quuQ::attR_TT-proD-Bba_B0034-Dasher-rnpB_T1-attL_CC (inverted) |
| SLOC085 | sLOC001 yqaB_argQ::attR_TT-proD-Bba_B0034-Dasher-rnpB_T1-attL_CC (inverted) |
| SLOC086 | sLOC001 frvA_rhaM::attR_TT-proD-Bba_B0034-Dasher-rnpB_T1-attL_CC (inverted) |
| SLOC087 | sLOC001 ybfC_ybfQ::attR_TT-proD-Bba_B0034-Dasher-rnpB_T1-attL_CC (inverted) |
| SLOC088 | sLOC001 rseX_yedS::attR_TT-proD-Bba_B0034-Dasher-rnpB_T1-attL_CC (inverted) |
| SLOC089 | sLOC001 ygcE_queE::attR_TT-proD-Bba_B0034-Dasher-rnpB_T1-attL_CC (inverted) |
| SLOC090 | sLOC001 frwA_frwC::attR_TT-proD-Bba_B0034-Dasher-rnpB_T1-attL_CC (inverted) |
| SLOC091 | sLOC001 ykgA_ykgQ::attR_TT-proD-Bba_B0034-Dasher-rnpB_T1-attL_CC (inverted) |
| SLOC092 | sLOC001 ybiJ_ybiI::attR_TT-proD-Bba_B0034-Dasher-rnpB_T1-attL_CC (inverted) |
| SLOC093 | sLOC001 yeeJ_yeeL::attR_TT-proD-Bba_B0034-Dasher-rnpB_T1-attL_CC (inverted) |
| SLOC094 | sLOC001 malM_yjbI::attR_TT-proD-Bba_B0034-Dasher-rnpB_T1-attL_CC (inverted) |
| SLOC095 | sLOC001 ykgH_betA::attR_TT-proD-Bba_B0034-Dasher-rnpB_T1-attL_CC (inverted) |
| SLOC096 | sLOC001 ymgF_ycgH::attR_TT-proD-Bba_B0034-Dasher-rnpB_T1-attL_CC (inverted) |
| SLOC097 | sLOC001 udk_yegE::attR_TT-proD-Bba_B0034-Dasher-rnpB_T1-attL_CC (inverted) |
| SLOC098 | sLOC001 dadX_cvrA::attR_TT-proD-Bba_B0034-Dasher-rnpB_T1-attL_CC (inverted) |
| SLOC099 | sLOC001 yffL_yffM::attR_TT-proD-Bba_B0034-Dasher-rnpB_T1-attL_CC (inverted) |
| SLOC100 | sLOC001 sibD_sibE::attR_TT-proD-Bba_B0034-Dasher-rnpB_T1-attL_CC (inverted) |
| SLOC101 | sLOC001 glpD_yzgL::attR_TT-proD-Bba_B0034-Dasher-rnpB_T1-attL_CC (inverted) |
| SLOC102 | sLOC001 yjiP_yjiR::attR_TT-proD-Bba_B0034-Dasher-rnpB_T1-attL_CC (inverted) |
| SLOC103 | SLOC009 (djlA_yabP) + pLys-M1 |
| SLOC104 | sLOCO11 (tyrV_tyrT) + pLys-M1 |
| SLOC105 | SLOC012 (ypjC_ileY) + pLys-M1 |
| SLOC106 | SLOC013 (yhiM_yhiN) + pLys-M1 |
| SLOC107 | SLOC014 (thrW_ykfN) + pLys-M1 |
| SLOC108 | SLOC017 (ileY_ygaQ) + pLys-M1 |
| SLOC109 | SLOC020 (ybfK_kdpE) + pLys-M1 |
| SLOC110 | SLOC021 (cspF_quuQ) + pLys-M1 |
| SL0C111 | SLOC022 (yqaB_argQ) + pLys-M1 |
| SLOC112 | SLOC023 (frvA_rhaM) + pLys-M1 |
| SLOC113 | SLOC025 (ybfC_ybfQ) + pLys-M1 |
| SLOC114 | SLOC026 (rseX_yedS) + pLys-M1 |
| SLOC115 | SLOC027 (ygcE_queE) + pLys-M1 |
| SLOC116 | SLOC028 (frwA_frwC) + pLys-M1 |
| SLOC117 | SLOC029 (ykgA_ykgQ) + pLys-M1 |
| SLOC118 | SLOC030 (ybiJ_ybiI) + pLys-M1 |
| SLOC119 | SLOC031 (yeeJ_yeeL) + pLys-M1 |
| SLOC120 | SLOC033 (malM_yjbI) + pLys-M1 |
| SLOC121 | SLOC034 (ykgH_betA) + pLys-M1 |
| SLOC122 | SLOC035 (ymgF_ycgH) + pLys-M1 |
| SLOC123 | SLOC036 (udk_yegE) + pLys-M1 |
| SLOC124 | SLOC040 (dadX_cvrA) + pLys-M1 |
| SLOC125 | SLOC041 (yffL_yffM) + pLys-M1 |
| SLOC126 | SLOC042 (sibD_sibE) + pLys-M1 |
| SLOC127 | SLOC045 (glpD_yzgL) + pLys-M1 |
| SLOC128 | SLOC046 (yjiP_yjiR) + pLys-M1 |

TABLE 2
Plasmid list

| Plasmid | Description |
|---|---|
| pLP | pSC101-repA-attP_TT-TetA-attP_CC-neo |
| plnt1 | pSC101-repA-lacI-pLac-PhiC31 (1) |
| pDasher | pSC101-repA-attB_CC-proD-Bba_B0034-Dasher-rnpB_T1-attB_TT-neo |
| pmCherry | pSC101-repA-attB_CC-proD-Bba_B0034-mCherry-rnpB_T1-attB_TT-neo |
| pmKate2_02 | pSC101-repA-attB_CC-proD-Bba_B0034-mKate2_02-rnpB_T1-attB_TT-neo |
| pDasherRV | pSC101-repA-attB_TT-proD-Bba_B0034-Dasher-rnpB_T1-attB_CC-neo (inverted) |
| pLys-M1 | Addgene plasmid #109382 (2) |

TABLE 3
Annotated nucleotide sequence of pLP (5250 bp DNA circular)

| Features | Description | Start | End |
|---|---|---|---|
| repA | repA protein | 37 | 987 |
| pSC101 | origin of replication | 1035 | 1257 |
| attP_TT | serine integrase attachment site | 1796 | 1845 |
| neo | neomycine phosphotransferase | 1968 | 3173 |
| attP_CC | serine integrase attachment site | 3366 | 3415 |
| Tn5 | kanamycin resistance | 3843 | 4637 |

TABLE 4
Annotated nucleotide sequence of pDasher (4428 bp DNA circular)

| Features | Description | Start | End |
|---|---|---|---|
| proD | promoter | 5 | 148 |
| Bba_B0034 | 5′-UTR | 171 | 182 |
| Dasher | coding sequence (proprietary sequence of ATUM, USA | 190 | 900 |
| rnpB_T1 | terminator | 905 | 986 |
| SpacerRightA | spacer | 991 | 1050 |
| attB_TT | serine integrase attachment site | 1155 | 1103 |
| repA | repA protein | 1398 | 2348 |
| pSC101 | origin of replication | 2396 | 2618 |
| neo | neomycine phosphotransferase | 4017 | 3223 |
| attB_CC | serine integrase attachment site | 4331 | 4279 |
| SpacerLeftA | spacer | 4369 | 4428 |

Media and Culture Conditions

The culture medium lysogeny broth (LB) (45) was used for precultures throughout the work. Lysogeny broth agar (LBA) is similarly composed with the addition of 12 g/L agar. For growth experiments measuring fluorescence a defined medium contained 2 g/L NH₄Cl, 5 g/L (NH₄)₂SO₄, 3 g/L KH₂PO₄, 7.3 g/L K₂HPO₄, 8.4 g/L MOPS, 0.5 g/L NaCl, 0.5 g/L MgSO₄.7H₂O, and 16.5 g/L glucose.H₂O, 1 ml/L trace element solution and 100 μL/L of a 0.967 g/L Na₂MoO₄.2H₂O molybdate solution. The trace element solution contained 3.6 g/L FeCl₂.4H₂O, 5 g/L CaCl₂.2H₂O, 1.3 g/L MnCl₂.2H₂O, 0.38 g/L CuCl₂.2H₂O, 0.5 g/L CoCl₂.6H₂O, 0.94 g/L ZnCl₂, 0.0311 g/L H₃BO₄, 0.4 g/L Na₂EDTA.2H₂O, 1.01 g/L thiamine. HCl. The defined medium was sterilized with a bottle top filter (Corning PTFE filter, 0.22 μm). Final antibiotic concentrations were as follows: spectinomycin (100 μg/mL), kanamycin (50 μg/mL), chloramphenicol (34 μg/mL) or tetracyline (10 μg/mL).

Next to the rich Luria Broth (LB), a minimal medium for shake flask (MMsf) and a minimal medium for fermentation (MMf) were used in the examples. Both minimal media use a trace element mix. Trace element mix consisted of 3.6 g/L FeCl2.4H20, 5 g/L CaCl2.2H20, 1.3 g/L MnCl2.2H20, 0.38 g/L CuCl2.2H20, 0.5 g/L CoCl2.6H20, 0.94 g/L ZnCl2, 0.0311 g/L H3B04, 0.4 g/L Na2EDTA.2H20 and 1.01 g/L thiamine.HCl. The molybdate solution contained 0.967 g/L Na2Mo04.2H20. The selenium solution contained 42 g/L Se02.

The Luria Broth (LB) medium consisted of 1% tryptone peptone (Difco, Erembodegem, Belgium), 0.5% yeast extract (Difco) and 0.5% sodium chloride (VWR, Leuven, Belgium).

Luria Broth agar (LBA) plates consisted of the LB media, with 12 g/L agar (Difco, Erembodegem, Belgium) added.

Minimal medium for shake flask experiments (MMsf) contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4.7H20. A carbon source chosen from, but not limited to glucose, fructose, maltose, glycerol and maltotriose, was used. The concentration was default 15 g/L, but this was subject to change depending on the experiment. 1 mL/L trace element mix, 100 μL/L molybdate solution, and 1 mL/L selenium solution. The medium was set to a pH of 7 with 1M KOH. Depending on the experiment lactose could be added as a precursor.

The minimal medium for fermentations contained 6.75 g/L NH4Cl, 1.25 g/L (NH4)2S04, 1.15 g/L KH2PO4 (low phosphate medium) or 2.93 g/L KH2PO4 and 7.31 g/L KH2PO4 (high phosphate medium), 0.5 g/L NaCl, 0.5 g/L MgSO4.7H20, a carbon source including but not limited to glucose, sucrose, fructose, maltose, glycerol and maltotriose, 1 mL/L trace element mix, 100 μL/L molybdate solution, and 1 mL/L selenium solution with the same composition as described above. Complex medium, e.g. LB, was sterilized by autoclaving (121° C., 21) and minimal medium (MMsf and MMf) by filtration (0.22 μm Sartorius). If necessary, the medium was made selective by adding an antibiotic (e.g. ampicillin (100mg/L), chloramphenicol (20 mg/L), carbenicillin (100mg/L), spectinomycin (40mg/L) and/or kanamycin (50mg/L)).

Chromosomal Integration using SIRE

Chromosomal integration of the fluorescent cassettes was done with Serine Integrase Recombinational Engineering (SIRE) (17). In brief, a landing pad with selectable marker tetA flanked with attP_{TT} and attP_{CC} was introduced in E. coli K-12 MG1655 using homologous recombination with the λ Red recombinase system (11). Second, the plasmid carrying the donor DNA flanked with complementary attB_{TT} and attB_{CC} sites was introduced and selected for. Next, vector pInt1 containing the PhiC31 integrase was introduced and selected for on spectinomycin while simultaneously expressing the integrase overnight with 0.4 mM IPTG (isopropyl-β-D-thiogalactopyranoside) induction on LBA plates. The genomically integrated donor DNA was checked with PCR (Dasher, mCherry or mKate2 cassette) and verified by Sanger sequencing for 10% of the strains (LGC Genomics, Germany).

Fluorescence Assays in Plate Reader

Bacterial cultures were inoculated 1% from an LB preculture started from single colony and incubated in Greiner Bio-One clear 96 well plates at 37° C. and 800 rpm. They were grown overnight in the defined medium described above, containing 2.2 g/L glucose.H₂O, which led to equal outgrowth due to carbon-limitation. Cultures were diluted 100-fold in fresh defined medium containing 16.5 g/L glucose.H₂O in Greiner Bio-One pClear black 96 well plates. Plates were grown in an incubation room of 37° C. containing two mtp-shakers (800 rpm), a robotic arm and a Tecan Spark 10 M microplate reader, performing measurements of Dasher (excitation (ex.), 486 nm; emission (em.), 532 nm), mCherry (ex., 575 nm; em., 625 nm), mKate2 (ex., 588 nm; em., 633 nm) and optical density (OD, 600 nm) every 30 min. Each experiment consisted of a minimum of three biological replicates. Fluorescence values were corrected for background fluorescence (E. coli K-12 MG1655) and OD₆₀₀ measurements and compared between strains at the start of the stationary phase. This point was calculated by the specific moment in the growth curve where the log(OD₆₀₀) deviates 20% from the linear fit of the maximum specific growth rate (46).

Statistical analyses were performed with a linear regression model of the package StatsModel for Python. The output can be found in Table 5.

TABLE 5
Statistical output for the linear regression model of the fluorescence
of our Dasher reporter cassette in function of the nett distance from
oriC. Analysis performed with the package StatsModel for Python.

OLS Regression Results

| Dep. Variable: | Dasher corr. OD and WT | | R-squared: | | 0.631 | |
|---|---|---|---|---|---|---|
| Model: | OLS | | Adj. R-squared: | | 0.623 | |
| Method: | Least Squares | | F-statistic: | | 82.1 | |
| No. Observations: | 50 | | Prob (F-statistic): | | 5.76E−12 | |
| Df Residuals: | 48 | | Log-Likelihood: | | −386.92 | |
| Df Model: | 1 | | AIC: | | 777.8 | |
| Covariance Type: | nonrobust | | BIC: | | 781.7 | |
| | coef | std err | t | P > \|t\| | [0.025 | 0.975] |
| const | 5752.7085 | 158.568 | 36.279 | 0.000 | 5433.887 | 6071.53 |
| net distance oriC | −1.1367 | 0.125 | −9.061 | 0.000 | −1.389 | −0.884 |

| | | | |
|---|---|---|---|
| Omnibus: | 7.454 | Durbin-Watson: | 1.796 |
| Prob (Omnibus): | 0.024 | Jarque-Bera (JB): | 7.115 |
| Skew: | −0.921 | Prob (JB): | 0.0285 |
| Kurtosis: | 3.144 | Cond. No. | 2.50E+03 |

Warnings

[1] Standard Errors assume that the covariance matrix of the errors is correctly specified.

[2] The condition number is large, 2.51e+03. This might indicate that there are strong multicollinearity or other numerical problems.

Flow cytometry

The plasmid pLys-M1 was transformed in strains containing the Dasher reporter cassette using heat shock (47). Bacterial cultures were inoculated 1% from an LB preculture and incubated in Greiner Bio-One clear 96 well plates at 37° C. and 800 rpm. They were grown overnight in the defined medium described above containing 2.2 g/L glucose.H₂O, which led to equal outgrowth due to carbon-limitation. Cultures were diluted 100-fold in fresh defined medium containing 2.2 g/L glucose.H₂O, with and without induction of 0.2% L-arabinose to express the VioB-mCherry reporter. Plates were grown at 37° C. and 800 rpm for 16 h after which cultures were diluted 1000× in phosphate-buffered saline (PBS) (48).

Cultures were analysed on a BD LSRFortessa™ Cell analyser with BD FACSDiva software. Calibration was done with BD™ Cytometer Setup and Tracking Beads. The blue (B530, 488 nm, filter 533/30) and yellow-green (Y610, 561 nm, filter 610/20) lasers were used for measurements of Dasher and VioB-mCherry respectively. Used parameters and PMT voltages were forward scatter (FSC: 334), side scatter (SSC: 370, with threshold value 500), blue laser (B530: 481) and yellow-green laser (V610: 670). FlowJo_V10 software was used to filter out cell debris and discriminate for single cells. Without induction the total amount of green fluorescent cells were considered and with induction calculation were done on cells which were red as well as green fluorescent.

Statistical analyses were performed using the package SciPy for python for the 26 strains containing the pLys-M1 plasmid, which were grown with and without induction of L-arabinose. Each condition was grown in threefold in defined medium which originated from the same LB preculture (n=3). Normality was assumed in all statistical tests. To determine if induction of the VioB-mCherry reporter resulted in lower genomic expression of the Dasher reporter, a paired one-sided t-test was performed with a 95% confidence interval. One-sided t-test were chosen to comply with the hypothesis that VioB-mCherry expression results in higher burden and thus can only result in lower genomic expression. Strains that were found to be significantly lower in Dasher fluorescence because of VioB-mCherry induction (p<0.05), were compared to each other with ANOVA (Tukey correction) using SPSS software to determine if these strains were equally influenced by the imposed burden.

Cultivation Conditions

A preculture of 96 well microtiter plate experiments was started from single colony on a LB plate, in 175 μL and was incubated for 8 h at 37° C. on an orbital shaker at 800 rpm. This culture was used as inoculum for a 96 well microtiter plate, with 175 μL MMsf medium by diluting 300×. These cultures in turn, were used as a preculture for the final experiment in a 96well plate, again by diluting 300×. The 96 well plate can either be microtiter plate, with a culture volume of 175 μL or a 24 well deepwell plate with a culture volume of 3 mL.

A preculture for shake flask experiments was started from a single colony on a LB-plate, in 5 mL LB medium and was incubated for 8 h at 37° C. on an orbital shaker at 200 rpm. From this culture, 1 mL was transferred to 100 mL minimal medium (MMsf) in a 500 mL shake flask and incubated at 37° C. on an orbital shaker at 200 rpm. This setup is used for shake flask experiments.

A shake flask experiment grown for 16 h could also be used as an inoculum for a bioreactor. 4% of this cell solution was to inoculate a 2L Biostat Dcu-B with a 4 L working volume, controlled by MFCS control software (Sartorius Stedim Biotech, Melsungen, Germany). Culturing condition were set to 37° C., 800 rpm stirring, and a gas flow rate of 1.5 L/min. The pH was controlled at 7 using 0.5 M H2S04 and 25% NH4OH. The exhaust gas was cooled. A 10% solution of silicone antifoaming agent was added when foaming raised during the fermentation.

Analytical Methods

Optical density

Cell density of the culture was frequently monitored by measuring optical density at 600 nm (Implen Nanophotometer NP80, Westburg, Belgium). Cell dry weight was obtained by centrifugation (10 min, 5000 g, Legend X1R Thermo Scientific, Belgium) of 20 g reactor broth in pre-dried and weighted falcons. The pellets were subsequently washed once with 20 mL physiological solution (9 g/L NaCl) and dried at 70° C. to a constant weight. To be able to convert OD₆₀₀ₙₘ measurements to biomass concentrations, a correlation curve of the OD₆₀₀ₙₘ to the biomass concentration was made.

Measurement of Cell Dry Weight

From a broth sample, 4×10 g was transferred to centrifuge tubes, the cells were spun down (5000g, 4° C., 5 min), and the cells were washed twice with 0.9% NaCl solution. The centrifuge tubes containing the cell pellets were dried in an oven at 70° C. for 48 h until constant weight. The cell dry weight was obtained gravimetrically; the tubes were cooled in a desiccator prior to weighing.

Liquid Chromatography

The concentration of carbohydrates like glucose, fructose, lactose, fucosylated human milk oligosaccharides (HMOs) and neutral HMOs . . . were determined with a Waters Acquity UPLC H-class system with an ELSD detector, using a Acquity UPLC BEH amide, 130 Å, 1.7 μm, 2.1 mm×50 mm heated at 35° C., using a 75/25 acetonitrile/water solution with 0.2% triethylamine (0.130 mL/min) as mobile phase.

Sialyllactose was quantified on the same machine, with the same column. The eluent however was modified to 75/25 acetonitrile/water solution with 1% formic acid. The flow rate was set to 0.130 mL/min and the column temperature to 35° C.

Sialic acid was quantified on the same machine, using the REZEX ROA column (300×7.8 mm ID). The eluent is 0.08% acetic acid in water. The flow rate was set to 0.5 mL/min and the column temperature to 65° C.

Yeast Strain Examples

Strains

Saccharomyces cerevisiae BY4742 (MATα, ura3Δ0, his3Δ1, leu2Δ0, lys2Δ0) was obtained from the Euroscarf culture collection. S. cerevisiae strains were stored at −80° C. in cryovials with 30% sterile glycerol in a 1:1 ratio mixture.

Media

Strains were grown on Synthetic Defined yeast medium with Complete Supplement Mixture (SD CSM) or CSM drop-out (e.g. SD CSM-Ura) containing 6.7 g.L⁻¹ Yeast Nitrogen Base without amino acids (YNB w/o AA, Difco), 20 g.L⁻¹ agar (Difco) (solid cultures), 22 g.L⁻¹ glucose monohydrate (Riedel-De Haen) and 0.79 g.L⁻¹ CSM or e.g. 0.77 g.L⁻¹ CSM-Ura (MP Biomedicals).

Cultivation Conditions

Yeast cultures were first inoculated from plate in 5 mL of the appropriate medium with an inoculation needle and incubated overnight at 30° C. and 200 rpm. In order to obtain single colonies as start material for the growth and production experiments, strains were plated on selective SD CSM plates and incubated for 2-3 days at 30° C. One colony was then picked and transferred to 5 mL medium. In order to obtain higher volume cultures, 2% (or higher) of the pre-culture was inoculated in 50-200 mL medium. These cultures were again incubated at 30° C. and 200 rpm. Growth experiments were conducted on Erlenmeyer scale (or on MTP for fluorescence measurements, see further).

Sampling Methodology

Samples of both the OD (0.2 mL) and the cellular and supernatant fraction (1 mL) of the culture were taken at regular time intervals for 2 to 5 days. The 1 mL sample was first centrifuged (10000 rpm, 5 minutes) after which the cell pellet and the supernatant were separated. Supernatant was stored at −20° C. for extracellular product analysis while the pellets were used for intracellular metabolite analysis. The cells were resuspended into 100 μL CelLytic Y Cell Lysis Reagent (Sigma) and acid-washed glass beads of 425-600 μm of diameter were added (Sigma). Next, the sample was vortexed for 1 minute at 4° C. and then put on ice for at least 30 seconds to cool down again. After repeating this cycle 10 times, the cells with beads were pelleted by centrifuging at 15000 rpm for 5 minutes. The supernatant was removed, filtered and stored in vials at −20° C.

Analytical Methods

Cell density of the culture was monitored by measuring optical density at 600 nm (Uvikom 922 spectrophotometer, BRS, Brussel, Belgium) or with the with the Biochrom Anthos Zenyth 340 Microtiterplate reader. To be able to convert OD₆₀₀ₙₘ measurements to biomass concentrations, a correlation curve of the OD₆₀₀ₙₘ to the biomass concentration was made.

To measure the expression level of fluorescent proteins, yeast strains were grown from cryovial and plated on selective SD CSM medium. Four colonies of the strains were selected and cultured in 150 μL selective SD CSM medium using a transparent 96-well plate (MTP, Greiner).

Afterwards, the plate was incubated at 30° C. and 800 rpm (Thermo scientific) for 48 hours until the stationary phase was reached. After 48 hours the colonies were grown in fresh selective SD CSM medium. In order to ensure that the growth of different strains starts at about the same level, a 150 times dilution was applied. Next, the plate was again incubated at 30° C. (with a range of variation of ±0.5° C.) in a multiplate reader (Infinite-200-PRO, Tecan). During incubation, every 15 minutes the following parameters were measured; (1) absorbance at 600 nm to evaluate growth, (2) measurement of the fluorescent signal.

Intracellular and extracellular product analysis was performed using Ultrahigh Performance Liquid Chromatography (UPLC) and detected using both mass spectrometry (MS) and an evaporative light scattering detector (ESLD). For example, separation of the samples was performed by an isocratic separation method using an Acquity UPLC BEH amide 1.7 μM column (Waters) at 35° C. As mobile phase, a solution composed out of 75% acetonitrile (ACN) with 0.2% triethyl amine (TEA) was used (1 mL.min⁻¹). When detection was performed by MS, the samples were ionized using a heated electrospray ionization (HESI) source and scanned in negative mode ranging from 100 m/z to 800 m/z.

Genetic Methods

Plasmids were maintained in the host E. coli DH5α (F⁻, φ80dlacZΔM15, Δ(lacZYA-argF)U169, deoR, recA1, endA1, hsdR17(rk⁻, mk⁺), phoA, supE44, λ⁻, thi-1, gyrA96, relA1).

Plasmids

Yeast expression plasmid p2a_2μ_10-5Lac12 available at the Laboratory of Industrial Biotechnology and Biocatalysis, UGent, Belgium was used to induce burden in Saccharomyces.

This plasmid contains an ampicillin resistance gene and a bacterial origin of replication to allow for selection and maintenance in E. coli. The plasmid further contains the 2 μ yeast ori and the Ura3 selection marker for selection and maintenance in yeast. Finally, the plasmid contains a lactose transporter expression cassette (SEQ ID 102). Plasmid p414-TEF1p-Cas9-CYC1t (Addgene #43802) and plasmid p426-SNR52p-gRNA.CAN1.Y-SUP4t (Addgene #43803) were used for CrispR-Cas9 mediated introduction of linear DNA at the loci under evaluation.

Linear Double-Stranded-DNA.

The linear ds-DNA amplicons were obtained by PCR using plasmid pJET_HRᵤ_22WcaG_33Gmd_54FT_HR_{d} or plasmid pJET_HRᵤ_pTDH3_yECitrine_tENO1_HR_{d}. These plasmids contain the transcription units for the 2′-FL production pathway (SEQ ID 103) or a transcription unit for a fluorescent marker (SEQ ID 104), respectively, flanked by 2 500 bp homology regions homologous to the locus under evaluation, at the multi-cloning site of the pJET Cloning vector (Thermoscientific). The primers used are homologous to the 5′ end of HRᵤ (forward primer) and the 3′ end of HR_{d} (reverse primer). PCR products were PCR-purified prior to transformation.

Transformations.

Plasmids and linear double stranded DNA were transformed using the method of Gietz (63).

### Example 2: Selection of the Locations

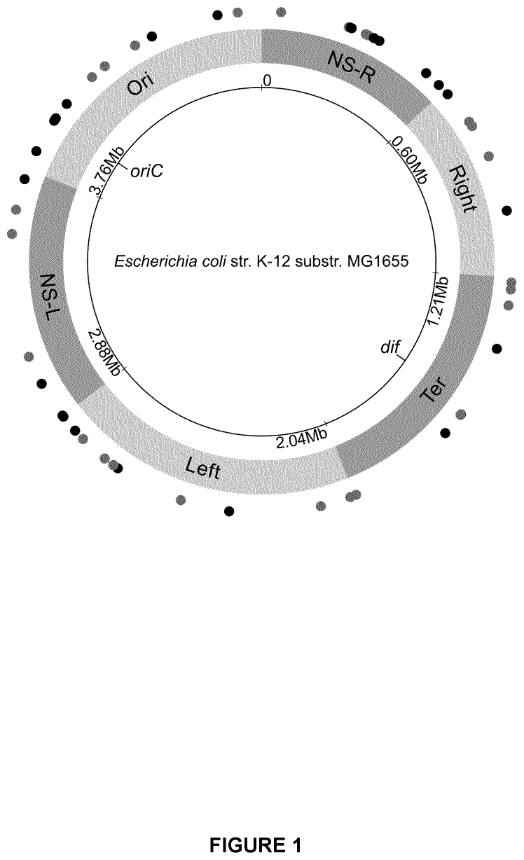

To investigate the influence of the chromosome position on the expression capacity of Escherichia coli several intergenic regions spread over the genome were selected. In this example, to avoid possible interactions with E. coli regulatory leader sequences, regions that contain promoters, 5′-UTRs, 3′-UTRs, transcription terminators, sigma factors, enhancers or silencers, were excluded (7, 9, 49, 50). Intergenic regions with substantial transcripts compared to their flanking sequences were omitted, since these can hold novel regulatory sequences (49). Genomic parts containing sRNAs and repetitive elements were also removed (49, 51). As an additional constraint, only intergenic regions of at least 200 bp in length were chosen, to simplify designs. Based on all these aspects, 74 intergenic locations were withheld. Of these 38 were chosen based on their spread over the macrodomains and non-structured regions of the E. coli genome (31) and on the orientation of the surrounding genes of the intergenic region. These also contain locations (partially) overlapping transcriptionally silenced (tsEPODs) or highly expressed extended protein occupancy domains (heEPODs) (28). To compare the data with currently existing literature on E. coli genomic expression, extra locations were included in our study. These are the intergenic locations lacZ_lacl, ycbW_ycbX, nupG_speC, asIB_asIA, atpl_gidB, yieN_trkD, ybbD_ylbG, essQ_cspB, and nth_ydgR (34-36). Last three regions were added because of the importance of the (surrounding) genes in E. coli research, these are ackA_pta (52), fucl_fucK (53), and xylB_xylA (54). The locations were named based on their neighbouring genes. The chosen 50 locations and their position on the E. coli genome are shown in FIG. 1, detailed information is included in Table 6.

TABLE 6
Detailed information on the 50 genomic locations
and their position on the E. coli genome.

| Location | Orientation | Macrodomain (5) | heEPODs (6) | tsEPODs (6) |
|---|---|---|---|---|
| djlA_yabP | Codirectional+ | R-NS | no overlap | no overlap |
| ylcI_nohD | Divergent | R-NS | no overlap | no overlap |
| tyrV_tyrT | Codirectional− | TER | internal | no overlap |
| ypjC_ileY | Codirectional− | LEFT | no overlap | internal |
| yhiM_yhiN | Convergent | L-NS | no overlap | internal |
| thrW_ykfN | Convergent | R-NS | no overlap | no overlap |
| entF_fepE | Codirectional+ | Right | no overlap | no overlap |
| ydaG_racR | Codirectional− | TER | no overlap | no overlap |
| ileY_ygaQ | Divergent | LEFT | no overlap | internal |
| dinD_yicG | Codirectional+ | ORI | no overlap | no overlap |
| ykfA_perR | Codirectional− | R-NS | no overlap | no overlap |
| ybfK_kdpE | Convergent | Right | no overlap | no overlap |
| cspF_quuQ | Convergent | TER | internal | no overlap |
| yqaB_argQ | Codirectional− | LEFT | internal | no overlap |
| frvA_rhaM | Codirectional− | ORI | no overlap | no overlap |
| insN_eyeA | Codirectional+ | R-NS | no overlap | no overlap |
| ybfC_ybfQ | Codirectional+ | Right | no overlap | internal |
| rseX_yedS | Codirectional+ | TER | no overlap | no overlap |
| ygcE_queE | Convergent | L-NS | no overlap | no overlap |
| frwA_frwC | Divergent | ORI | no overlap | no overlap |
| ykgA_ykgQ | Divergent | R-NS | no overlap | internal |
| ybiJ_ybiI | Codirectional− | Right | no overlap | no overlap |
| yeeJ_yeeL | Convergent | LEFT | no overlap | no overlap |
| ygeF_ygeG | Codirectional+ | L-NS | no overlap | internal |
| malM_yjbI | Codirectional+ | ORI | no overlap | internal |
| ykgH_betA | Codirectional− | R-NS | no overlap | internal |
| ymgF_ycgH | Codirectional+ | TER | no overlap | internal |
| udk_yegE | Divergent | LEFT | no overlap | no overlap |
| ygeK_ygeN | Codirectional− | L-NS | no overlap | internal |
| yjcS_alsK | Codirectional− | ORI | no overlap | internal |
| yahK_yahL | Codirectional+ | R-NS | no overlap | internal |
| dadX_cvrA | Convergent | TER | no overlap | no overlap |
| yffL_yffM | Codirectional+ | LEFT | no overlap | no overlap |
| sibD_sibE | Codirectional− | L-NS | no overlap | no overlap |
| yjhV_fecE | Convergent | ORI | no overlap | no overlap |
| yfjQ_yfjR | Codirectional+ | LEFT | no overlap | no overlap |
| glpD_yzgL | Convergent | L-NS | internal | no overlap |
| yjiP_yjiR | Convergent | ORI | no overlap | no overlap |
| lacZ_lacI | Codirectional− | R-NS | no overlap | no overlap |
| ycbW_ycbX | Convergent | Right | no overlap | no overlap |
| nupG_speC | Convergent | L-NS | no overlap | no overlap |
| aslB_aslA | Convergent | ORI | no overlap | no overlap |
| atpI_gidB | Codirectional− | ORI | right overlap | no overlap |
| yieN_trkD | Divergent | ORI | no overlap | no overlap |
| ybbD_ylbI | Codirectional+ | R-NS | no overlap | right overlap |
| essQ_cspB | Codirectional− | TER | left overlap | no overlap |
| nth_ydgR | Codirectional+ | TER | no overlap | no overlap |
| ackA_pta | Knock−out | LEFT | no overlap | no overlap |
| fucI_fucK | Codirectional+ | L-NS | no overlap | no overlap |
| xylB_xylA | Codirectional− | L-NS | no overlap | no overlap |

### Example 3: Effect of Genomic Location on Expression

Strain Construction

To examine the expression strength of the genomic locations, a fluorescent protein (FP) was inserted in the intergenic regions selected in example 2 using SIRE (17). The only exception is ackA_pta, where a double knockout is made instead of integration in the intergenic region. The genomic homologies used to integrate the landing pad onto the genome are listed in Table 7. For the constructs, the insulated promoter proD (41) with the Bba_B0034 ribosome binding site (http://parts.igem.org/) and high efficient terminator mpB_T1 (42) are used. Additionally, biologically neutral 60 bp spacers designed according to Casini et al. (56) and 53 bp attB sites are surrounding the construct, which altogether results in a fluorescent protein expression cassette insulated from genomic context (41, 57, 58).

TABLE 7
Genomic homologies used to integrate the landing
pad onto the genome (SEQ ID Nos 2 to 101)

| Location | Homology 1 (5′-3′) | Homology 2 (5′-3′) |
|---|---|---|
| djIA_yabP | CTCAATGCACGGTTTACGGGAGGGGTTCTGT | AGACGTAAAAATATAATTCCGCTCGTCGTA |
| | AGGTTTTATCGCGTTGACC | AAGCTCTCAACCTTAAGCAG |
| ylcl_nohD | TAGATGATAATTATTATCATTTTGTGGGTCC | CCGGAAAATTTTCATAAATAGCGAAAACCC |
| | TTTCCGGCGATCCGACAGG | GCGAGGTCGCCGCCCCGTAA |
| tyrV_tyrT | TTCGTCGCTTCGCTCCTCACCCTTCGGGCCG | CGGGGAAGGGTGAGAACCTTCGACTAAGGT |
| | TTGCCTGTGGCAACGTTCT | TCGATTCGAGCGAAAGCGAG |
| ypjC_ileY | AGTAGTAGATGTTTAAGGCGTGGCAGAGACA | TCGCTCACTGATGATAAGTGAGTACCACAA |
| | TTTCATCCTTACTCTACGG | CCAATGTATGTAGAACAATG |
| yhiM_yhiN | CAGCAAAGTTACTGTTTTTTTCAACCTGTTC | CATGCTTAATATAAGGTGGATGGAAAGGTG |
| | ATATTTCATAAAGATCTGG | ATTGAAAACTCACTCAGTGG |
| thrW_ykfN | TCTTAATGTAACAGCTGGTGTAAGTAAATTC | AAGGATGTATAGTGAGCGAAGCCCTATCAG |
| | TATCAACGAAGATCAATCT | GCCTTTTTGGTCAGTAGATA |
| entF_fepE | TTGATTTATAGGTTTGATGAATATTTCTCTT | AGTTGGTGATAATTATCCGAAGCTGAAGTT |
| | AAATAGAGTGAATGTTGCA | TGTAAATTCCTTCCACTGAA |
| ydaG_racR | ACCACTGCCTGGTAACTCGAAGTATTGCCCG | AGCCTATTGACAATCAATTAGGCATTACCT |
| | GCGTTCTGTGGGGCGGGGT | ATAGTTCCAGCATACCACCC |
| ileY_ygaQ | GTATCTAATAATATAACTTTATTACATTAGC | ATTGCTATACGAAGTTTATTTTTATGGAGT |
| | TGAAGAGTTTTCGCATCAT | GAAAAGTAACAGATATCATA |
| dinD_yicG | TTTTCCCCCTCAGTTTTAACCTATTTTTTCT | GTTATGTGAAATCGCTATTTTCTGTAGCAG |
| | TATGCATTTTCTCAGACAA | AGATGCATTCTTCTGACTTC |
| ykfA_perR | AGGCAGCTGCGCGACTGCTGGCTCAGGCAAT | AAGGTGTATCACGGCGGCTCATACTCTCAA |
| | GAATGAGTTATAATAGCAG | TAAATCCCTGTTAGTAAATG |
| ybfK_kdpE | CAATAAAAAATGATCAATCTTAATTTATTTA | TTTTTATCTTAAACAACACACAAAAATAAC |
| | ATGATGAGCTTTTTACTCA | AATTCAATATTTTATATTAC |
| cspF_quuQ | GTTTAGGGACATTGTACTGGAAGAAAACATT | CTCATCCCGGGACTCATGTCTGTTAACTTA |
| | TTAAACATCAGGCAAATAA | TTATTTAGCTGGTGACTTGG |
| yqaB_argQ | TGGATAAAGGAGTTATTTAGAAATGAGATAT | CCCGAAGGGCGAACGTCAGTGAGTCATCCT |
| | TTTTGAAGGAAATTTTTTG | CCCGGATGCACCATCTTCTC |
| frvA_rhaM | TGAAAGGTCAGATTTGCGGAGTAATGCACAT | ATTGTGAGTAAATCACAAAAATAATGAATA |
| | AATGGTTATTTAAATAAAC | ACCCATTAATGATTCATGTG |
| insN_eyeA | TGCCCGCAGGGTGATGTAACCCGCTGACAAC | CATGTTCTTCAACCTTTCAGTACTTAACCT |
| | GGGGATTGAGGCGAGATCA | TGAGGATCATCTCGGCTTAG |
| ybfC_ybfQ | TTTATTTTGCGTTCCATTTGCAGGGAAAGAT | CAATAAGTAGTATCTCAATTGTTGAACTTA |
| | CACGTAACGCTACTTTTTT | AAATTCGAATTATTTAGTAC |
| rseX_yedS | ATTTTCATGAATATTTATATTTAGAATTCAT | GATTACATGTAACAAATGTATTTAAAAGAT |
| | AATTATGAATTATATTAAA | ATCAAAATGTTTCTAATCTA |
| ygcE_queE | GGTGGTTTATCCCCGCTGGCGCGGGGAACTC | GAAAACAGGTGTTCCCCGCGCCAGCGGGGA |
| | GACAGAACGGCCTCAGTAG | TAAACCGGAGCCTGACGAGA |
| frwA_frwC | CAATTTGCGACGCGTCTCACAAGACGCTGTT | ACTTTTGTAATATCAGTACAAAAATGCGAT |
| | TTGCGGCATGCTTCCGGTT | CCGCCTCATAACTTGCGATA |
| ykgA_ykgQ | CCGAAAATAGAGAGGTTTCAGTCCTACATTA | GTCTACGTTAAAACGTAACCTCAAAGTAGT |
| | TTAATGAATTTTTTGCATA | ATGTGGATTTTGATATCACT |
| ybiJ_ybil | AAATCGAAGAGAATTGACCGCCTTGTTCAAA | CGGTATAAAACAAGTTCATAAGTACAACAA |
| | TAAATTGATTGATATCTAA | ATAAATGGTTTATCAGTAGG |
| yeeJ_yeeL | CACAGAAAATGAATAAATAAAAATGCGGCAC | AAACCAGCCTTTAGATCAAAGCAGTACTCA |
| | CGCCAGAATCGCGTTCGAT | CCGAAAATGATCATAGTCAC |
| ygeF_ygeG | GATGTTATTAGTTTGTAGTGAACAGTACTTT | TATATTTATCTTTTTTAAATTATGAGTTTT |
| | TACCAATAATGAAAAATAT | AAGCTTGCATTGCTTATGGT |
| malM_yjbl | TCCTTCCTGGGATATGAGCGATTTTTTATAG | GCGAAAGGAAAAGAATCTCTGATAAGGCAT |
| | TAACTCACTTCTTCTTCAC | TGAGATAATGGATATTCTTA |
| ykgH_betA | AGGAATGTTCGGGTTAAATATCAGCAAAAAG | GGGGGACCGAATCCTTATATAAACACTGAG |
| | CCCGCATCATGAATACTGG | GTAACTCTCATGCTTCATAT |
| ymgF_ycgH | GCAACTATTAACAATTTTGATGTCGAAGAGT | GCATTATCATTTTTCACCTTATTTTCATGA |
| | TATTTGTTAAACAAAATCG | CATTGATCACTTTGAGGTGA |
| udk_yegE | CGCGCTCAGAGTTAATTGTTGACAAAGAATT | ATAATTTGCGCAACTGCGTTTAACATTTTT |
| | CCCGGGGGCAAATTACGTT | TACCTTACATAAAACTGATC |
| ygeK_ygeN | ATTATAAGCAAAATCCAAAGAATACATTGAT | GATTTTTTAATGCCTGTGGTATTTTTTTAC |
| | GAAATAATAATGAAATATA | GCAAAAATTTTATTTTTAAT |
| yjcS_alsK | TTGCGACTTTAATAAGTGGAAGTGTGAGCGG | ATTTTCTGCAATGATAGTTTTACTGTAATT |
| | AACGCGCCATTTTATTAGG | TTCCCTCTTCAGCACAAATG |
| yahK_yahL | CGAAATAATATCAAAGTAGCAGTAAAACCTA | TCGCTCATAACTAACGTGTGAAGTATTGTG |
| | TAACGTAAATTTAAATTGT | TACTGGAGGGCGTTAATTTA |
| dadX_cvrA | AACCTGAACTCACCGCACAGGCGTTCTACAT | GCTCCATCAAGGGTAAAGCGTGATTTATCT |
| | AAAACGCTTACGCTTCATT | GAAGTCGAGTTCGAGTCAAC |
| yffL_yffM | TTTTTAGCCTCCCGGTCGGTCATAGAGAGTC | AGCATGGTTAATGCTCGCAACCAGCCGACC |
| | GCCTAGAGTTAAACAGAAG | TATCAGGCGGCGAAATAATT |
| sibD_sibE | AAAAGCCGGGGATTTTTTATATCTGCGTTCC | AGGCAATTTTGCCTTCCCCGAGCGGTCACG |
| | GCTAAAAGGTGCAAATGCT | CAAAACGCTGCAACGTCCTG |
| yjhV_fecE | CCTGAAATCTAAACTTAGTCATGTCACGTTT | GCTTAACGGACATTTCTGTATAACCCTTAC |
| | TTGGGTTTCTAAAATTTTA | GGCAACGAAAAACGCGAAGT |
| yfp_yfjR | TCGTGTGCCTCAATCCCCCGGTTATAGCTTT | GGCGGACAGGGTATGGACAACGCAGAAACT |
| | TAACCCCCGTTACATCTGG | ATTTTTTATTTCTGCAAAAG |
| glpD_yzgL | AGGCCTACGTGGTTTATGCAATATATTGAAT | TTGACAAAGTGCGCTTTGTTCATGCCGGAT |
| | TTGCATGGTCTTGTAGGCC | GCGACGTGAACGTCTTATCT |
| yjiP_yjiR | TATTGAACTTTAAAGATTTTTGTAGACCTGG | ATCGCCACGTTCCAGCCTGAATTAAGCAAA |
| | TCAGGCGTTCACATGGCAT | GTACGCTTTGTTCATGCCGG |
| lacZ_lacl | CCGAGTTAACGCCATCAAAAATAATTCGCGT | CATTAATGCAGCTGGCACGACAGGTTTCCC |
| | CTGGCCTTCCTGTAGCCAG | GACTGGAAAGCGGGCAGTGA |
| ycbW_ycbX | TGAAACCGCAGGTTAATGTTGACAGCTTCAG | TTCTTTGCTGTAGCTGTGTACCGAAGACTG |
| | CCTCGAACAGGCAGTCTAA | CACTTAAGTTGGCGCGTTAG |
| nupG_speC | ATAAACACGTTCGTGTCCCGACAGGCACACA | GTAAGAATAAAAAAAACGGGTCACCTTCTG |
| | GACGGTTAGCCACTAATTA | GCGACCCGTTTTTCTTTGCG |
| asIB_asIA | TGTAGGCTGGATAAGATGCGTCAGCATCGCA | AATATCCACCACGCGCGCAGATTAAATCTG |
| | TCCGGCAAAGGCAGATCTC | ACTAAGCCGGCGCTATCGCT |
| atpl_gidB | CAAAAAGCGGTCAAATTATACGGTGCGCCCC | ATAACGTGGCTTTTTTTGGTAAGCAGAAAA |
| | CGTGATTTCAAACAATAAG | TAAGTCATTAGTGAAAATAT |
| yieN_trkD | TGGCGTCCTTTCGTCAAAAGTTCTGCGTAAA | GTATGCACGATTAACGGCAAAATCGTACTC |
| | TTGCGAGTATAGACGTTTC | CTAAATGCGGCCACATTAAC |
| ybbD_ylbl | CTGAGAAAAGACATGTCGGCTATTGTGTAAA | TTCTATGTAAACTCTCTGACTGTTCATTTT |
| | GCCATATAGCTCAGACGAT | ATTTGTTGTTTCAGGGTCGG |
| essQ_cspB | ATGGTGCAATATGTTTGAAAAGATCGGAGTC | GATAATTACGGCGTGATTTTGAGTTTTTAC |
| | TACGGGGTAGTTTTGACAG | GTTCTGACATAGGCTTTTCC |
| nth_ydgR | TTAACGTCAATGATGCCATTGCTTAGCGTTA | GATAGTCCAGTTTCTGAAAAATAGCCAGTG |
| | TCATCAGGTAATCCGTTTG | TAATGTTTTGTAGGTCAATA |
| ackA_pta | CTATGGCTCCCTGACGTTTTTTTAGCCACGT | TTATTTCCGGTTCAGATATCCGCAGCGCAA |
| | ATCAATTATAGGTACTTCC | AGCTGCGGATGATGACGAGA |
| fucl_fucK | TTACTCCCTGATGTGATGCCCGGTCGCTCCG | GCTCCTGCAATATAGCCGGATAACATTGCT |
| | GCTACCGGGCCTGAACAAG | TATCCGGCTAACCACTCTTG |
| xylB_xylA | TATCCCGATATACATATCGATCGTTCCTTAA | TGTTCGACAAATAACGGCTAACTGTGCAGT |
| | AAAAATGCCCGGTATCGCT | CCGTTGGCCCGGTTATCGGT |

Selection of Reporter Cassette

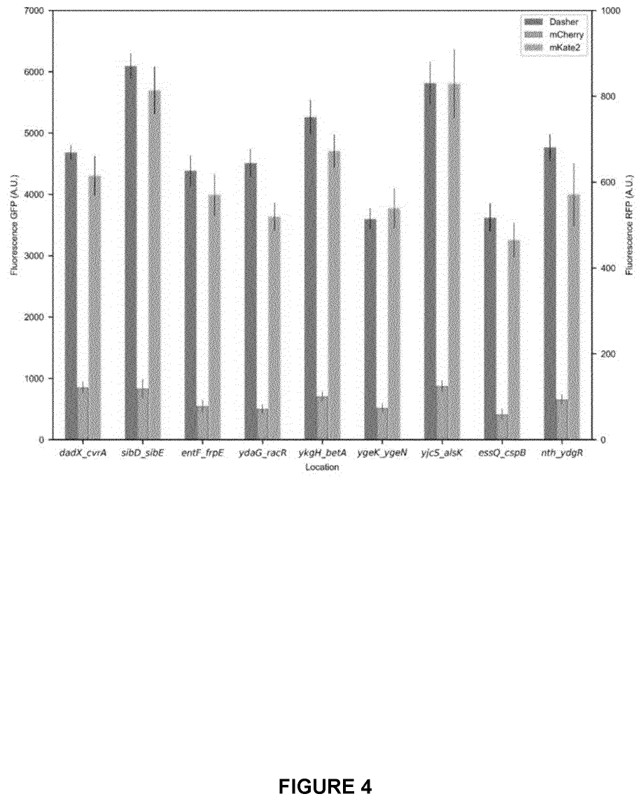

To avoid a low signal-to-noise ratio, long maturation time, or fast saturation of measurements, different candidate FPs such as sfGFP (38), mCherry (40), mKate2 (39) and several Paintbox proteins (ATUM, USA) were tested on plasmid level of which a green fluorescent protein (Dasher) and two red fluorescent proteins (mCherry and mKate2) were withheld (data not shown). To validate their suitability on the genome, the expression cassettes were inserted on nine different locations. Their fluorescent output is given in FIG. 4. From the data can be deduced that the output is comparable for the three FPs, meaning that the coding sequence of these three FPs and the protein itself has little influence on the relationship between the locations. Despite Dasher and mKate2 having a similar higher signal-to-noise ratio than mCherry, Dasher was chosen as the reporter FP for it has a maturation time close to zero (data not shown).

Based on the above, we designed fluorescent expression cassettes so that specific local effects on gene expression, originating from surrounding genes, transcriptional read through and influence from transcription factors, are eliminated. This design was validated by obtaining a 1 on 1 correlation between the fluorescence output on the forward and reverse incorporation of our Dasher GFP reporter cassette (data not shown).

Evaluation of Genomic Expression

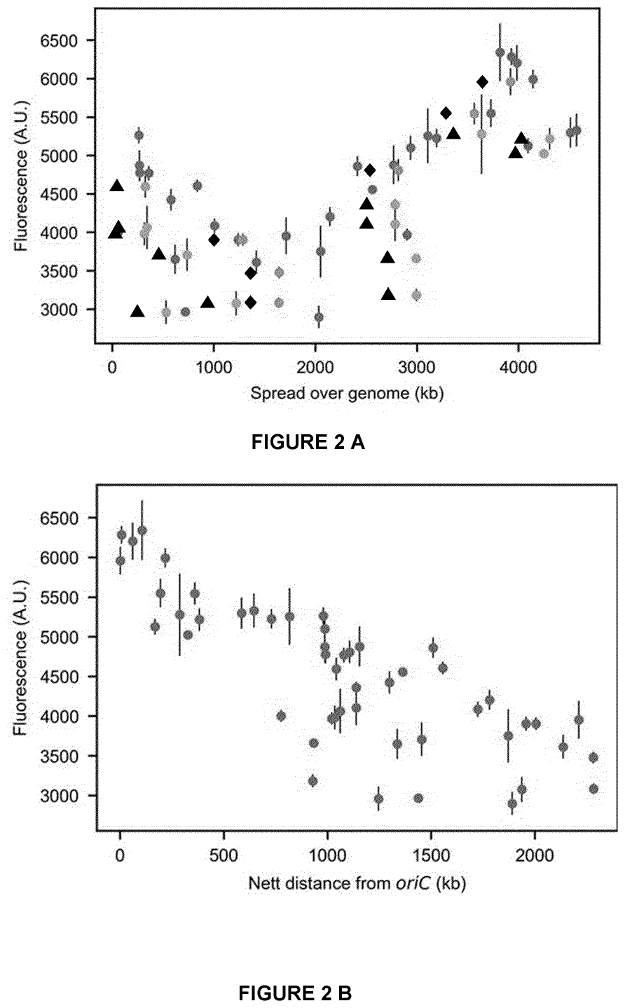

The Dasher reporter cassette was integrated at 50 different locations according to the description above. GFP fluorescence measurements were taken during the entire growth phase, whereupon the values at the start of the stationary phase were used to compare all strains. In FIG. 2a, the fluorescence of the reporter cassette in function of the genomic position is shown. A 2.22-fold difference in expression was observed between the highest expressing strain (dinD_yicG) and the lowest expressing strain (rseX_yedS). Using the genomic location as a tool for expression optimization is thus limited especially in comparison with the fold increase typically seen in promoter-RBS libraries. A trend is seen where fluorescence decreases towards 1600 kb and again rises towards 4000 kb which coincides with the locations of dif and oriC respectively. When calculating the nett distance from oriC, this trend is clearly confirmed (FIG. 2b) and is in accordance to literature where the gene dosage effect was also seen. Six of the chosen intergenic regions are within a highly expressed extended protein occupancy domain (heEPOD) (indicated with diamonds in FIGS. 2a) and 12 are within a transcriptionally silent EPOD (tsEPOD) (indicated with triangles in FIG. 2a) (28). Also for these regions the gene dosage effect seems to apply as heEPODs near dif result in a lower fluorescence than heEPODS near oriC. However, the fluorescence remains in the same size order, independent from the presence of tsEPODs or heEPODs.

### Example 4: Burden Effect

Experimental Set-Up

Heterologous gene expression can be a significant burden for cells. Often this burden is not caused by the specific heterologous sequences, but by a general resource depletion in the cells. Therefore, Ceroni et al. developed a fluorescence-based method to measure the gene expression capacity of bacterial cells in real time (61). They developed several plasmids, including pLys-M1, a medium copy plasmid with a strong promoter-RBS expression system, coding for a fusion protein of VioB and mCherry which imposes a significant burden upon the cell. By using a ‘capacity monitor’, an FP expression cassette inserted on a fixed position on the genome, they were able to quantify burden by measuring red and green fluorescence.

To check whether some locations are influenced by imposed burden, we transformed pLys-M1 in our 26 strains expressing the Dasher reporter cassette on different locations spread over the genome. As Ceroni et al. reported ‘escape mutants’, cells not able to express the fluorescent protein VioB-mCherry because of mutations in the plasmid during the growth cycle, we changed our experimental set-up from plate readers to flow cytometry to look at single-cell level. Cultures were then grown with and without induction of the VioB-mCherry cassette (on the burden plasmid pLys-M1) and the genomic green fluorescence of both cases were compared (see material and methods in Example 1).

### Flow Cytometry Outcome

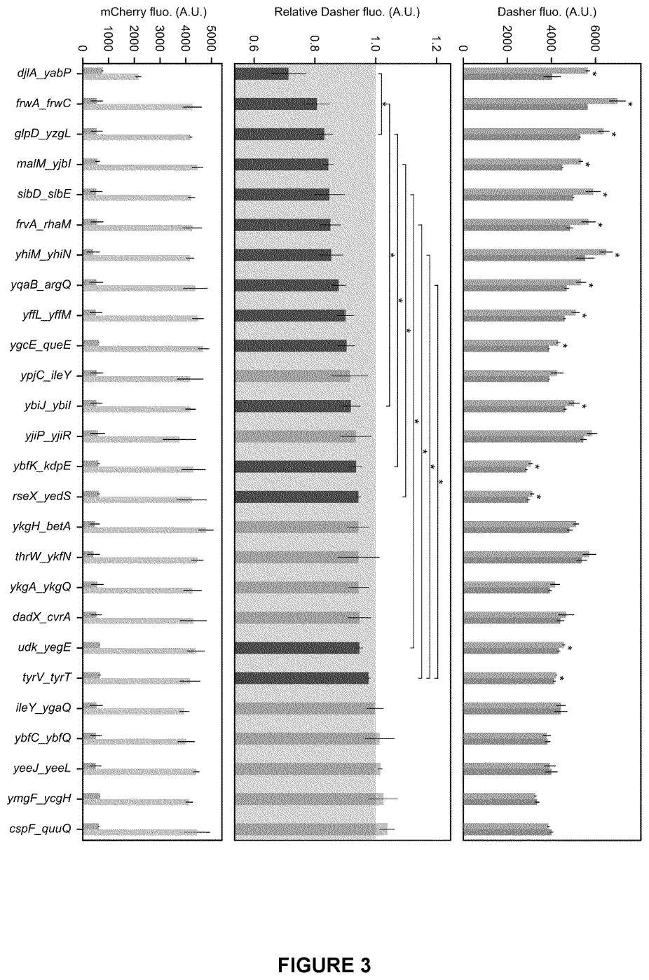

In FIG. 3, the outcome of the flow cytometry experiment is summarized. In the top barplot, fluorescence of the Dasher reporter cassette is shown with and without induction of VioB-mCherry. Strains indicated with an * have a significantly diminished (p<0.05) fluorescent output of the reporter cassette due to the imposed burden. This was determined using a paired one-sided t-test (p-values can be found in Table 8).

TABLE 8
p-values of the paired one-sided t-test for the 26 locations
to check if the green fluorescence output is significantly
diminished on imposing burden by pLys-M1

| Location | p-value | Rejecting null hypothesis |
|---|---|---|
| dadX_cvrA | 0.076 | False |
| rseX_yedS | 0.005 | True |
| djlA_yabP | 0.006 | True |
| tyrV_tyrT | 0.011 | True |
| ypjC_ileY | 0.078 | False |
| yhiM_yhiN | 0.011 | True |
| thrW_ykfN | 0.141 | False |
| ileY_ygaQ | 0.461 | False |
| ybfK_kdpE | 0.018 | True |
| cspF_quuQ | 0.058 | False |
| yqaB_argQ | 0.009 | True |
| frvA_rhaM | 0.012 | True |
| frwA_frwC | 0.012 | True |
| ykgA_ykgQ | 0.060 | False |
| ybiJ_ybiI | 0.030 | True |
| yeeJ_yeeL | 0.040 | False |
| malM_yjbI | 0.003 | True |
| ykgH_betA | 0.058 | False |
| udk_yegE | 0.007 | True |
| yffL_yffM | 0.016 | True |
| sibD_sibE | 0.024 | True |
| glpD_yzgL | 0.007 | True |
| yjiP_yjiR | 0.084 | False |
| ybfC_ybfQ | 0.357 | False |
| ygcE_queE | 0.016 | True |
| ymgF_ycgH | 0.223 | False |

The middle barplot in FIG. 3 shows the relative Dasher fluorescence of induction over control. All strains that were found to be significantly diminished in Dasher fluorescence upon induction of VioB-mCherry (p<0.05), were compared with each other with ANOVA (Tukey correction) to determine if these strains were equally influenced by the imposed burden (p-values can be found in Table 8). In the bottom barplot the fluorescence of the VioB-mCherry cassette is given, with and without induction.

TABLE 9
Output generated from SPSS software on the ANOVA analysis with
Tukey correction for determining significant differences between
strains influenced by burden. Values indicated with an * show
that the mean difference is significant at the 5% level.

| | | | | | 95% Confidence Interval | |
|---|---|---|---|---|---|---|
| (I) | (J) | Mean Diff. | Std. | p- | Lower | Upper |
| Location | Location | (I − J) | Error | value | Bound | Bound |
| djlA_yabP | frwA_rwC | −0.0937 | 0.02628 | 0.066 | −0.1906 | 0.0031 |
| | glpD_yzgL | −.1178* | 0.02628 | 0.007 | −0.2147 | −0.021 |
| | malM_yjbI | −.1307* | 0.02628 | 0.002 | −0.2276 | −0.0339 |
| | sibD_sibE | −.1346* | 0.02628 | 0.001 | −0.2314 | −0.0377 |
| | frvA_rhaM | −.1380* | 0.02628 | 0.001 | −0.2349 | −0.0412 |
| | yhiM_yhiN | −.1401* | 0.02628 | 0.001 | −0.2369 | −0.0432 |
| | yqaB_argQ | −.1650* | 0.02628 | 0 | −0.2619 | −0.0682 |
| | yffL_yffM | −.1876* | 0.02628 | 0 | −0.2845 | −0.0908 |
| | ygcE_queE | −.1905* | 0.02628 | 0 | −0.2874 | −0.0937 |
| | ybiJ_ybiI | −.2054* | 0.02628 | 0 | −0.3023 | −0.1086 |
| | ybfK_kdpE | −.2222* | 0.02628 | 0 | −0.3191 | −0.1254 |
| | rseX_yedS | −.2295* | 0.02628 | 0 | −0.3264 | −0.1327 |
| | udk_yegE | −.2334* | 0.02628 | 0 | −0.3303 | −0.1366 |
| | tyrV-tyrT | −.2633* | 0.02628 | 0 | −0.3601 | −0.1664 |
| frwA_frwC | djlA_yabP | 0.0937 | 0.02628 | 0.066 | −0.0031 | 0.1906 |
| | glpD_yzgL | −0.0241 | 0.02628 | 1 | −0.121 | 0.0727 |
| | malM_yjbI | −0.037 | 0.02628 | 0.98 | −0.1338 | 0.0598 |
| | sibD_sibE | −0.0409 | 0.02628 | 0.956 | −0.1377 | 0.056 |
| | frvA_rhaM | −0.0443 | 0.02628 | 0.922 | −0.1411 | 0.0526 |
| | yhiM_yhiN | −0.0463 | 0.02628 | 0.895 | −0.1432 | 0.0505 |
| | yqaB_argQ | −0.0713 | 0.02628 | 0.344 | −0.1681 | 0.0256 |
| | yffL_yffM | −0.0939 | 0.02628 | 0.065 | −0.1907 | 0.003 |
| | ygcE_queE | −0.0968 | 0.02628 | 0.05 | −0.1936 | 0.0001 |
| | ybiJ_ybiI | −.1117* | 0.02628 | 0.013 | −0.2086 | −0.0149 |
| | ybfK_kdpE | −.1285* | 0.02628 | 0.002 | −0.2253 | −0.0316 |
| | rseX_yedS | −.1358* | 0.02628 | 0.001 | −0.2327 | −0.039 |
| | udk_yegE | −.1397* | 0.02628 | 0.001 | −0.2366 | −0.0429 |
| | tyrV_tyrT | −.1696* | 0.02628 | 0 | −0.2664 | −0.0727 |
| glpD_yzgL | djlA_yabP | .1178* | 0.02628 | 0.007 | 0.021 | 0.2147 |
| | frwA_frwC | 0.0241 | 0.02628 | 1 | −0.0727 | 0.121 |
| | malM_yjbI | −0.0129 | 0.02628 | 1 | −0.1097 | 0.084 |
| | sibD_sibE | −0.0167 | 0.02628 | 1 | −0.1136 | 0.0801 |
| | frvA_rhaM | −0.0202 | 0.02628 | 1 | −0.117 | 0.0767 |
| | yhiM_yhiN | −0.0222 | 0.02628 | 1 | −0.1191 | 0.0746 |
| | yqaB_argQ | −0.0472 | 0.02628 | 0.882 | −0.144 | 0.0497 |
| | yffL_yffM | −0.0698 | 0.02628 | 0.376 | −0.1666 | 0.0271 |
| | ygcE_queE | −0.0727 | 0.02628 | 0.317 | −0.1695 | 0.0242 |
| | ybiJ_ybiI | −0.0876 | 0.02628 | 0.109 | −0.1844 | 0.0093 |
| | ybfK_kdpE | −.1044* | 0.02628 | 0.025 | −0.2012 | −0.0075 |
| | rseX_yedS | −.1117* | 0.02628 | 0.013 | −0.2085 | −0.0148 |
| | udk_yegE | −.1156* | 0.02628 | 0.009 | −0.2125 | −0.0188 |
| | tyrV_tyrT | −.1454* | 0.02628 | 0 | −0.2423 | −0.0486 |
| malM_yjbI | djlA_yabP | .1307* | 0.02628 | 0.002 | 0.0339 | 0.2276 |
| | frwA_frwC | 0.037 | 0.02628 | 0.98 | −0.0598 | 0.1338 |
| | glpD_yzgL | 0.0129 | 0.02628 | 1 | −0.084 | 0.1097 |
| | sibD_sibE | −0.0039 | 0.02628 | 1 | −0.1007 | 0.093 |
| | frvA_rhaM | −0.0073 | 0.02628 | 1 | −0.1041 | 0.0896 |
| | yhiM_yhiN | −0.0093 | 0.02628 | 1 | −0.1062 | 0.0875 |
| | yqaB_argQ | −0.0343 | 0.02628 | 0.99 | −0.1311 | 0.0626 |
| | yffL_yffM | −0.0569 | 0.02628 | 0.685 | −0.1537 | 0.04 |
| | ygcE_queE | −0.0598 | 0.02628 | 0.615 | −0.1566 | 0.0371 |
| | ybiJ_ybiI | −0.0747 | 0.02628 | 0.278 | −0.1716 | 0.0221 |
| | ybfK_kdpE | −0.0915 | 0.02628 | 0.079 | −0.1883 | 0.0054 |
| | rseX_yedS | −.0988* | 0.02628 | 0.042 | −0.1957 | −0.002 |
| | udk_yegE | −.1027* | 0.02628 | 0.03 | −0.1996 | −0.0059 |
| | tyrV_tyrT | −.1326* | 0.02628 | 0.002 | −0.2294 | −0.0357 |
| sibD_sibE | djlA_yabP | .1346* | 0.02628 | 0.001 | 0.0377 | 0.2314 |
| | frwA_frwC | 0.0409 | 0.02628 | 0.956 | −0.056 | 0.1377 |
| | glpD_yzgL | 0.0167 | 0.02628 | 1 | −0.0801 | 0.1136 |
| | malM_yjbI | 0.0039 | 0.02628 | 1 | −0.093 | 0.1007 |
| | frvA_rhaM | −0.0034 | 0.02628 | 1 | −0.1003 | 0.0934 |
| | yhiM_yhiN | −0.0055 | 0.02628 | 1 | −0.1023 | 0.0914 |
| | yqaB_argQ | −0.0304 | 0.02628 | 0.997 | −0.1273 | 0.0664 |
| | yffL_yffM | −0.053 | 0.02628 | 0.773 | −0.1499 | 0.0438 |
| | ygcE_queE | −0.0559 | 0.02628 | 0.708 | −0.1528 | 0.0409 |
| | ybiJ_ybiI | −0.0709 | 0.02628 | 0.353 | −0.1677 | 0.026 |
| | ybfK_kdpE | −0.0876 | 0.02628 | 0.109 | −0.1845 | 0.0092 |
| | rseX_yedS | −0.095 | 0.02628 | 0.059 | −0.1918 | 0.0019 |
| | udk_yegE | −.0989* | 0.02628 | 0.042 | −0.1957 | −0.002 |
| | tyrV_tyrT | −.1287* | 0.02628 | 0.002 | −0.2256 | −0.0319 |
| frvA_rhaM | djlA_yabP | .1380* | 0.02628 | 0.001 | 0.0412 | 0.2349 |
| | frwA_frwC | 0.0443 | 0.02628 | 0.922 | −0.0526 | 0.1411 |
| | glpD_yzgL | 0.0202 | 0.02628 | 1 | −0.0767 | 0.117 |
| | malM_yjbI | 0.0073 | 0.02628 | 1 | −0.0896 | 0.1041 |
| | sibD_sibE | 0.0034 | 0.02628 | 1 | −0.0934 | 0.1003 |
| | yhiM_yhiN | −0.0021 | 0.02628 | 1 | −0.0989 | 0.0948 |
| | yqaB_argQ | −0.027 | 0.02628 | 0.999 | −0.1239 | 0.0698 |
| | yffL_yffM | −0.0496 | 0.02628 | 0.841 | −0.1465 | 0.0472 |
| | ygcE_queE | −0.0525 | 0.02628 | 0.785 | −0.1493 | 0.0444 |
| | ybiJ_ybiI | −0.0674 | 0.02628 | 0.429 | −0.1643 | 0.0294 |
| | ybfK_kdpE | −0.0842 | 0.02628 | 0.142 | −0.181 | 0.0127 |
| | rseX_yedS | −0.0915 | 0.02628 | 0.079 | −0.1884 | 0.0053 |
| | udk_yegE | −0.0954 | 0.02628 | 0.057 | −0.1923 | 0.0014 |
| yhiM_yhiN | djlA_yabP | .1401* | 0.02628 | 0.001 | 0.0432 | 0.2369 |
| | frwA_frwC | 0.0463 | 0.02628 | 0.895 | −0.0505 | 0.1432 |
| | glpD_yzgL | 0.0222 | 0.02628 | 1 | −0.0746 | 0.1191 |
| | malM_yjbI | 0.0093 | 0.02628 | 1 | −0.0875 | 0.1062 |
| | sibD_sibE | 0.0055 | 0.02628 | 1 | −0.0914 | 0.1023 |
| | frvA_rhaM | 0.0021 | 0.02628 | 1 | −0.0948 | 0.0989 |
| | yqaB_argQ | −0.025 | 0.02628 | 1 | −0.1218 | 0.0719 |
| | yffL_yffM | −0.0476 | 0.02628 | 0.876 | −0.1444 | 0.0493 |
| | ygcE_queE | −0.0504 | 0.02628 | 0.826 | −0.1473 | 0.0464 |
| | ybiJ_ybiI | −0.0654 | 0.02628 | 0.477 | −0.1622 | 0.0315 |
| | ybfK_kdpE | −0.0821 | 0.02628 | 0.166 | −0.179 | 0.0147 |
| | rseX_yedS | −0.0895 | 0.02628 | 0.094 | −0.1863 | 0.0074 |
| | udk_yegE | −0.0934 | 0.02628 | 0.067 | −0.1902 | 0.0035 |
| | tyrV_tyrT | −.1232* | 0.02628 | 0.004 | −0.2201 | −0.0264 |
| yqaB_argQ | djlA_yabP | .1650* | 0.02628 | 0 | 0.0682 | 0.2619 |
| | frwA_frwC | 0.0713 | 0.02628 | 0.344 | −0.0256 | 0.1681 |
| | glpD_yzgL | 0.0472 | 0.02628 | 0.882 | −0.0497 | 0.144 |
| | malM_yjbI | 0.0343 | 0.02628 | 0.99 | −0.0626 | 0.1311 |
| | sibD_sibE | 0.0304 | 0.02628 | 0.997 | −0.0664 | 0.1273 |
| | frvA_rhaM | 0.027 | 0.02628 | 0.999 | −0.0698 | 0.1239 |
| | yhiM_yhiN | 0.025 | 0.02628 | 1 | −0.0719 | 0.1218 |
| | yffL_yffM | −0.0226 | 0.02628 | 1 | −0.1195 | 0.0742 |
| | ygcE_queE | −0.0255 | 0.02628 | 0.999 | −0.1223 | 0.0714 |
| | ybiJ_ybiI | −0.0404 | 0.02628 | 0.96 | −0.1373 | 0.0564 |
| | ybfK_kdpE | −0.0572 | 0.02628 | 0.678 | −0.154 | 0.0397 |
| | rseX_yedS | −0.0645 | 0.02628 | 0.498 | −0.1614 | 0.0323 |
| | udk_yegE | −0.0684 | 0.02628 | 0.406 | −0.1653 | 0.0284 |
| | tyrV_tyrT | −.0983* | 0.02628 | 0.044 | −0.1951 | −0.0014 |
| yffL_yffM | djlA_yabP | .1876* | 0.02628 | 0 | 0.0908 | 0.2845 |
| | frwA_frwC | 0.0939 | 0.02628 | 0.065 | −0.003 | 0.1907 |
| | glpD_yzgL | 0.0698 | 0.02628 | 0.376 | −0.0271 | 0.1666 |
| | malM_yjbI | 0.0569 | 0.02628 | 0.685 | −0.04 | 0.1537 |
| | sibD_sibE | 0.053 | 0.02628 | 0.773 | −0.0438 | 0.1499 |
| | frvA_rhaM | 0.0496 | 0.02628 | 0.841 | −0.0472 | 0.1465 |
| | yhiM_yhiN | 0.0476 | 0.02628 | 0.876 | −0.0493 | 0.1444 |
| | yqaB_argQ | 0.0226 | 0.02628 | 1 | −0.0742 | 0.1195 |
| | ygcE_queE | −0.0029 | 0.02628 | 1 | −0.0997 | 0.094 |
| | ybiJ_ybiI | −0.0178 | 0.02628 | 1 | −0.1147 | 0.079 |
| | ybfK_kdpE | −0.0346 | 0.02628 | 0.989 | −0.1314 | 0.0623 |
| | rseX_yedS | −0.0419 | 0.02628 | 0.947 | −0.1388 | 0.0549 |
| | udk_yegE | −0.0458 | 0.02628 | 0.902 | −0.1427 | 0.051 |
| | tyrV_tyrT | −0.0757 | 0.02628 | 0.261 | −0.1725 | 0.0212 |
| ygcE_queE | djlA_yabP | .1905* | 0.02628 | 0 | 0.0937 | 0.2874 |
| | rwA_frwC | 0.0968 | 0.02628 | 0.05 | −0.0001 | 0.1936 |
| | glpD_yzgL | 0.0727 | 0.02628 | 0.317 | −0.0242 | 0.1695 |
| | malM_yjbI | 0.0598 | 0.02628 | 0.615 | −0.0371 | 0.1566 |
| | sibD_sibE | 0.0559 | 0.02628 | 0.708 | −0.0409 | 0.1528 |
| | frvA_rhaM | 0.0525 | 0.02628 | 0.785 | −0.0444 | 0.1493 |
| | yhiM_yhiN | 0.0504 | 0.02628 | 0.826 | −0.0464 | 0.1473 |
| | yqaB_argQ | 0.0255 | 0.02628 | 0.999 | −0.0714 | 0.1223 |
| | yffL_yffM | 0.0029 | 0.02628 | 1 | −0.094 | 0.0997 |
| | ybiJ_ybiI | −0.0149 | 0.02628 | 1 | −0.1118 | 0.0819 |
| | ybfK_kdpE | −0.0317 | 0.02628 | 0.995 | −0.1286 | 0.0651 |
| | rseX_yedS | −0.039 | 0.02628 | 0.969 | −0.1359 | 0.0578 |
| | udk_yegE | −0.0429 | 0.02628 | 0.937 | −0.1398 | 0.0539 |
| | tyrV_tyrT | −0.0728 | 0.02628 | 0.314 | −0.1696 | 0.0241 |
| ybiJ_ybiI | djlA_yabP | .2054* | 0.02628 | 0 | 0.1086 | 0.3023 |
| | frwA_frwC | .1117* | 0.02628 | 0.013 | 0.0149 | 0.2086 |
| | glpD_yzgL | 0.0876 | 0.02628 | 0.109 | −0.0093 | 0.1844 |
| | malM_yjbI | 0.0747 | 0.02628 | 0.278 | −0.0221 | 0.1716 |
| | sibD_sibE | 0.0709 | 0.02628 | 0.353 | −0.026 | 0.1677 |
| | frvA_rhaM | 0.0674 | 0.02628 | 0.429 | −0.0294 | 0.1643 |
| | yhiM_yhiN | 0.0654 | 0.02628 | 0.477 | −0.0315 | 0.1622 |
| | yqaB_argQ | 0.0404 | 0.02628 | 0.96 | −0.0564 | 0.1373 |
| | yffL_yffM | 0.0178 | 0.02628 | 1 | −0.079 | 0.1147 |
| | ygcE_queE | 0.0149 | 0.02628 | 1 | −0.0819 | 0.1118 |
| | ybfK_kdpE | −0.0168 | 0.02628 | 1 | −0.1136 | 0.0801 |
| | rseX_yedS | −0.0241 | 0.02628 | 1 | −0.121 | 0.0727 |
| | udk_yegE | −0.028 | 0.02628 | 0.999 | −0.1249 | 0.0688 |
| | tyrV_tyrT | −0.0579 | 0.02628 | 0.662 | −0.1547 | 0.039 |
| ybfK_kdpE | djlA_yabP | .2222* | 0.02628 | 0 | 0.1254 | 0.3191 |
| | frwA_frwC | .1285* | 0.02628 | 0.002 | 0.0316 | 0.2253 |
| | glpD_yzgL | .1044* | 0.02628 | 0.025 | 0.0075 | 0.2012 |
| | malM_yjbI | 0.0915 | 0.02628 | 0.079 | −0.0054 | 0.1883 |
| | sibD_sibE | 0.0876 | 0.02628 | 0.109 | −0.0092 | 0.1845 |
| | frvA_rhaM | 0.0842 | 0.02628 | 0.142 | −0.0127 | 0.181 |
| | yhiM_yhiN | 0.0821 | 0.02628 | 0.166 | −0.0147 | 0.179 |
| | yqaB_argQ | 0.0572 | 0.02628 | 0.678 | −0.0397 | 0.154 |
| | yffL_yffM | 0.0346 | 0.02628 | 0.989 | −0.0623 | 0.1314 |
| | ygcE_queE | 0.0317 | 0.02628 | 0.995 | −0.0651 | 0.1286 |
| | ybiJ_ybiI | 0.0168 | 0.02628 | 1 | −0.0801 | 0.1136 |
| | rseX_yedS | −0.0073 | 0.02628 | 1 | −0.1042 | 0.0895 |
| | udk_yegE | −0.0112 | 0.02628 | 1 | −0.1081 | 0.0856 |
| | tyrV_tyrT | −0.0411 | 0.02628 | 0.954 | −0.1379 | 0.0558 |
| rseX_yedS | djlA_yabP | .2295* | 0.02628 | 0 | 0.1327 | 0.3264 |
| | frwA_frwC | .1358* | 0.02628 | 0.001 | 0.039 | 0.2327 |
| | glpD_yzgL | .1117* | 0.02628 | 0.013 | 0.0148 | 0.2085 |
| | malM_yjbI | .0988* | 0.02628 | 0.042 | 0.002 | 0.1957 |
| | sibD_sibE | 0.095 | 0.02628 | 0.059 | −0.0019 | 0.1918 |
| | frvA_rhaM | 0.0915 | 0.02628 | 0.079 | −0.0053 | 0.1884 |
| | yhiM_yhiN | 0.0895 | 0.02628 | 0.094 | −0.0074 | 0.1863 |
| | yqaB_argQ | 0.0645 | 0.02628 | 0.498 | −0.0323 | 0.1614 |
| | yffL_yffM | 0.0419 | 0.02628 | 0.947 | −0.0549 | 0.1388 |
| | ygcE_queE | 0.039 | 0.02628 | 0.969 | −0.0578 | 0.1359 |
| | ybiJ_ybiI | 0.0241 | 0.02628 | 1 | −0.0727 | 0.121 |
| | ybfK_kdpE | 0.0073 | 0.02628 | 1 | −0.0895 | 0.1042 |
| | udk_yegE | −0.0039 | 0.02628 | 1 | −0.1008 | 0.0929 |
| | tyrV_tyrT | −0.0338 | 0.02628 | 0.991 | −0.1306 | 0.0631 |
| udk_yegE | djlA_yabP | .2334* | 0.02628 | 0 | 0.1366 | 0.3303 |
| | frwA_frwC | .1397* | 0.02628 | 0.001 | 0.0429 | 0.2366 |
| | glpD_yzgL | .1156* | 0.02628 | 0.009 | 0.0188 | 0.2125 |
| | malM_yjbI | .1027* | 0.02628 | 0.03 | 0.0059 | 0.1996 |
| | sibD_sibE | .0989* | 0.02628 | 0.042 | 0.002 | 0.1957 |
| | frvA_rhaM | 0.0954 | 0.02628 | 0.057 | −0.0014 | 0.1923 |
| | yhiM_yhiN | 0.0934 | 0.02628 | 0.067 | −0.0035 | 0.1902 |
| | yqaB_argQ | 0.0684 | 0.02628 | 0.406 | −0.0284 | 0.1653 |
| | yffL_yffM | 0.0458 | 0.02628 | 0.902 | −0.051 | 0.1427 |
| | ygcE_queE | 0.0429 | 0.02628 | 0.937 | −0.0539 | 0.1398 |
| | ybiJ_ybiI | 0.028 | 0.02628 | 0.999 | −0.0688 | 0.1249 |
| | ybfK_kdpE | 0.0112 | 0.02628 | 1 | −0.0856 | 0.1081 |
| | rseX_yedS | 0.0039 | 0.02628 | 1 | −0.0929 | 0.1008 |
| | tyrV_tyrT | −0.0298 | 0.02628 | 0.997 | −0.1267 | 0.067 |
| tyrV_tyrT | djlA_yabP | .2633* | 0.02628 | 0 | 0.1664 | 0.3601 |
| | frwA_frwC | .1696* | 0.02628 | 0 | 0.0727 | 0.2664 |
| | glpD_yzgL | .1454* | 0.02628 | 0 | 0.0486 | 0.2423 |
| | malM_yjbI | .1326* | 0.02628 | 0.002 | 0.0357 | 0.2294 |
| | sibD_sibE | .1287* | 0.02628 | 0.002 | 0.0319 | 0.2256 |
| | frvA_rhaM | .1253* | 0.02628 | 0.003 | 0.0284 | 0.2221 |
| | yhiM_yhiN | .1232* | 0.02628 | 0.004 | 0.0264 | 0.2201 |
| | yqaB_argQ | .0983* | 0.02628 | 0.044 | 0.0014 | 0.1951 |
| | yffL_yffM | 0.0757 | 0.02628 | 0.261 | −0.0212 | 0.1725 |
| | ygcE_queE | 0.0728 | 0.02628 | 0.314 | −0.0241 | 0.1696 |
| | ybiJ_ybiI | 0.0579 | 0.02628 | 0.662 | −0.039 | 0.1547 |
| | ybfK_kdpE | 0.0411 | 0.02628 | 0.954 | −0.0558 | 0.1379 |
| | rseX_yedS | 0.0338 | 0.02628 | 0.991 | −0.0631 | 0.1306 |
| | udk_yegE | 0.0298 | 0.02628 | 0.997 | −0.067 | 0.1267 |

From FIG. 3 can be deduced that genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH and cspF_quuQ are not significantly influenced by imposed burden, making them excellent choices to insert pathway genes that require stable expression. Expression can even be tuned since they all have a distinct strength. On the other hand, locations djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT are highly diminished in genomic expression due to the imposed burden. Although generally stable genomic expression is preferred, this can be interesting for the integration of pathway genes since the expression can be adjusted to the burden that is imposed on the cell.

It is to be noted that prior to flow cytometry analysis, the OD₆₀₀ of the cultures was measured (after 16 h incubation at 37° C. and 800 rpm). All cultures had OD₆₀₀ values of approximately 0.62, except for the strain containing djIA_yabP::Dasher which had OD₆₀₀ values of 0.262±0.022 for three replicates. Also on FIG. 3 can be seen that VioB-mCherry values for this strain are significantly lower than for the others, and this strain is more diminished in Dasher fluorescence than any other. It is important to realize that flow cytometry shows the fluorescence of single cells, meaning that the lower mCherry values cannot be assigned to the lower OD values. A more likely hypothesis is that this strain suffers from high amounts of burden, resulting in slower growth and less production of FPs. Based on the above, it can be said with certainty that location djlA_yabP is strongly influenced by environmental changes and that no stable expression can be obtained.

### Example 5: Effect of Loci on Expression Strength of a Heterologous Gene

The loci described in example 4 have been applied to tune the expression strength of a heterologous gene or pathway. Said expression tuning is of importance in the context of pathway optimization in synthetic biology. A high expression locus can debottleneck the pathway flux towards a specific bioproduct. The expression strength of each locus is given in the FIG. 10. Improving expression of a heterologous gene hence may be tuned by means of a chromosomal locus, for instance highest expression in FIG. 10 will be accomplished at the dinD_yicG locus.

### Example 6: Tuning a Biological Production Pathway by means of Burden Sensitive Genetic Loci

A burden sensitive chromosomal locus allows the introduction of a genetic feedback loop in the biological system. Said feedback loop is accomplished by introducing one gene or a set of genes of the biological pathway that is non-rate limiting at a burden sensitive chromosomal locus and another gene or set of genes of said biological pathway at another locus or plasmid so that it imposes a metabolic burden.

### Example 7: Tuning a Biological Production Pathway by means of Burden Sensitive Genetic Loci

As another example the influx of toxic substrates can be taken. For instance, the synthesis of lactose based oligosaccharide relies on lactose influx through the lactose permease gene. The construction of an overexpression strain of lactose permease in yeasts and bacteria is described in WO2016075243. Unlimited influx of lactose becomes quickly toxic to the cell when accumulating intracellular. By introducing the lactose permease gene at a metabolic burden sensitive locus, a feedback loop is created when burden starts occurring which then reduces the gene expression of said lactose permease.

### Example 8: Tuning a Lactose Permease Expression by means of Burden Sensitive Genetic Loci

The construction of an overexpression strain of lactose permease in yeasts and bacteria is described in WO2016075243. Said lactose permease is introduced with the genetic engineering method described in example 1 at the loci djlA_yabP and frwA_frwC in an E. coli cell. The expression of lactose permease is modulated with increasing lactose influx, by increasing lactose concentration in the growth medium. Modification of the lactose by means of a transferase (for instance the fucosylation of lactose as described in WO2012007481 and WO2013087884 or the sialylation of lactose as described in WO2018122225) decreases burden, increasing expression of the lactose permease and increases lactose influx in accordance to the pathway capacity. Accumulation of lactose in the cell increases burden, and reduces lactose influx in accordance to the pathway capacity.

### Example 9: The Production of a Fucosylated Oligosaccharide in E. coli

An E. coli strain was constructed by the heterologous introduction of genes encoding for the GDP-fucose biosynthesis pathway. Said genes code for the enzymes mannose-6-phosphate isomerase, phosphomannomutase, mannose-1-phosphate guanylyltransferase, GDP-mannose 4,6-dehydratase, GDP-L-fucose synthase. Said genes were introduced in at least one of the loci described in example 6, the loci locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH or cspF_quuQ. The fucosyltransferase is overexpressed with a strong promoter UTR selected (Nat Methods. 2013 April;10(4):354-60) or by induction on another locus on the chromosome or on a plasmid, imposing burden on the cell due to overexpression. Said burden does not change the expression of the GDP-fucose pathway genes.

### Example 10: Production of Sialic Acid in Escherichia coil

This example provides an Escherichia coli strain capable of producing N-acetylneuraminate (sialic acid).

A strain capable of accumulating glucosamine-6-phosphate using sucrose as a carbon source was further engineered to allow for N-acetylneuraminate production. The base strain overexpresses a sucrose phosphorylase from Bifidobacterium adolescentis (BaSP), a fructokinase from Zymomonas mobilis (Zmfrk), a mutant fructose-6-P-aminotransferase (EcglmS*54, as described by Deng et al. (Biochimie 88, 419-429 (2006))). To allow for sialic acid production the operons nagABCDE, nanATEK and manXYZ were disrupted. BaSP, Zmfrk and EcglmS*54 were introduced on a burden insensitive locus as described in example 11. These modifications were done as described in example 1.

In this strain, the biosynthetic pathway for producing sialic acid was implemented by overexpressing a glucosamine-6-P-aminotransferase from Saccharomyces cerevisiae (ScGNA1), an N-acetylglucosamine-2-epimerase from Bacteroides ovatus (BoAGE) (the use of these genes are described in WO2018122225). Similar to the BaSP, Zmfrk and EcglmS gene these genes were introduced on the chromosome at a burden insensitive locus or burden sensitive chromosomal loci.

The gene coding for sialic acid synthase from Campylobacter jejuni (CjneuB) was overexpressed on a plasmid so that it posed a burden on the cell. When introducing the biosynthetic pathway genes on a burden insensitive locus, the overexpression of CjneuB has minimal effect the biosynthetic pathway activity. When introducing one or more of the biosynthetic pathway genes on a burden sensitive locus, e.g. djlA_yabP and frwA_frwC, the pathway activity reduced, which leads to reduced production.

The strain was cultured as described in example 1 (materials and methods). Briefly, a 5 mL LB preculture was inoculated and grown overnight at 37° C. This culture was used as inoculum in a shake flask experiment with 100 mL medium which contains 10 g/L sucrose and was made as described in example 1. Regular samples were taken and analysed as described in example 1. The same organism also produces N-acetylneuraminate based on glucose, maltose or glycerol as carbon source.

### Example 11: Production of 6′-Sialyllactose in Escherichia coli

Another example according to present invention is the use of the method and strains for the production of 6′-sialyllactose.

The strain of example 12 was further modified by introducing the genes NmneuA and Pdbst, are expressed from a plasmid, together with CjneuB. This plasmid is pCX-CjneuB-NmneuA-Pdbst (the use of these genes are described in W02018122225). Said strain is inoculated as a preculture consisting of 5m1 LB medium as described in example 1. After growing overnight at 37° C. in an incubator. 1% of this preculture is inoculated in a shake flask containing 100 ml medium (MMsf) containing 10 g/l sucrose as carbon source and 10 g/l lactose as precursor. The strain is grown for 300 h at 37° C.

This strain produces quantities of 6′-sialyllactose and similar to example 10, when introducing the biosynthetic pathway genes on a burden insensitive locus, the overexpression of described plasmid has minimal effect the biosynthetic pathway activity. When introducing one or more of the biosynthetic pathway genes on a burden sensitive locus, e.g. djlA_yabP and frwA_frwC, the overexpression of the described plasmid reduced the pathway activity, which leads to reduced production.

### Example 12: Burden Resistant Loci Evaluated by Fluorescent Output in Saccharomyces cerevisiae

Using CrispR-Cas9 methodology, the transcription unit for expression of a fluorescence marker, such as, but not limited to, yCitrine, was introduced at several loci in the genome of Saccharomyces cerevisiae. Upon expression of a protein causing burden to Saccharomyces cerevisiae, such as, but not limited to the LAC12 transporter, from the yeast high copy 2 μ plasmid, burden on the genome was evaluated by measuring yCitrine fluorescence. Fluorescence levels were clearly influenced by the expression of the LAC12 transporter. The effect was different for the expression cassettes integrated at different loci. At some loci, fluorescence was lower, at others it was not affected.

### Example 13: Burden Resistant Loci Evaluated by HMO Production in Saccharomyces cerevisiae

Using CrispR-Cas9 methodology, the transcription units for expression of a production pathway of interest, such as, but not limited to, transcription units for the 2′-FL production pathway, was introduced at several loci in the genome of Saccharomyces cerevisiae. Upon expression of a protein causing burden to Saccharomyces cerevisiae, such as, but not limited to the LAC12 transporter, from the yeast high copy 2 μ plasmid, burden on the genome was evaluated by measuring 2′-FL production. Production levels were clearly influenced by the expression of the LAC12 transporter. The effect was different for the expression cassettes integrated at different loci. At some loci, production was lower, at others it was not affected.

### Example 14

Another exemplary embodiment of the present invention is the metabolic tuning of the expression of a heterologous gene or set of genes in a transgenic plant. The integration of a gene or set of genes encoding for a protein or the production of a bioproduct at a burden sensitive chromosomal location allows the reduction of expression of said gene or set of genes when the plant is exposed to unfavourable conditions for the plant such as but not limited to drought stress, water stress, heat stress, pest stress and/or cold stress. Said expression reduction allows the plant to survive unfavourable conditions easier. When the stress condition has passed, the expression of said gene or set of genes is restored to its normal level. Said tuning of expression is specifically applicable for transgenic plants that have difficulty to survive stress conditions when expressing a transgenic gene or set of genes.

### Example 15

Another exemplary embodiment of the present invention is also found for a plant wherein the introduction of a gene or set of genes is done on a burden insensitive or stable expression location in the chromosome. The integration of a gene or set of genes encoding for a protein or the production of a bioproduct at such a location in the chromosome, ensures expression in stress conditions such as but not limited to drought stress, water stress, heat stress, pest stress and/or cold stress. Such transformants keep on producing a protein or bioproduct at the same level over different environmental conditions, reducing the impact of environmental conditions on product yield. Further, such transformant can also comprise a heterologous gene providing e.g. a heat resistant or pest resistant gene which preferably is still produced under the burden or stress and enabling the plant to overcome such stress period rather unaffected.

### Example 16

A fluorescent GFP marker is introduced at different genome locations of rice plant cells by means of the method described by Nandy et al. (BMC Biotechnology 2015 15:93). The plants that have been modified with GFP at different chromosomal locations are exposed to several stress conditions such as drought, heat, cold and the GFP expression is measured. The GFP is measured by means of microscopy or by ELISA as described by Agnelo Furtado et al. (Plant Biotechnology Journal, 6, 679-693) or by qPCR. The expression of the GFP is compared with an unstressed control to assess the expression stability of the chromosomal locus.

### REFERENCES

1. Chen, X., Zhou, L., Tian ,K., Kumar, A., Singh, S., Prior, B. A. and Wang, Z. (2013) Metabolic engineering of Escherichia coli: A sustainable industrial platform for bio-based chemical production. Biotechnol. Adv., 31,1200-1223.

2. Becker, J. and Wittmann, C. (2016) Systems metabolic engineering of Escherichia coli for the heterologous production of high value molecules—a veteran at new shores. Curr. Opin. Biotechnol., 42,178-188.

3. Sauer, M., Porro, D., Mattanovich, D. and Branduardi, P. (2008) Microbial production of organic acids: expanding the markets. Trends Biotechnol., 26,100-108.

4. Lee, J. H., Jung, S. C., Bui, L. M., Kang, K. H., Song, J. J. and Kim, S. C. (2013) Improved Production of L-Threonine in Escherichia coli by Use of a DNA Scaffold System. Appl. Environ. Microbiol., 79,774-782.

5. Rodriguez, A., Martínez, J. A., Flores, N., Escalante, A., Gosset, G. and Bolivar, F. (2014) Engineering Escherichia coli to overproduce aromatic amino acids and derived compounds. Microb. Cell Fact., 13, 126.

6. Baumgärtner, F., Conrad, J., Sprenger, G. A. and Albermann, C. (2014) Synthesis of the human milk oligosaccharide lacto-N-tetraose in metabolically engineered, plasmid-free E. coli. Chembiochem, 15, 1896-1900.

7. Gama-Castro, S., Jiménez-Jacinto, V., Peralta-Gil, M., Santos-Zavaleta, A., Peñaloza-Spinola, M. I., Contreras-Moreira, B., Segura-Salazar, J., Muñiz-Rascado, L., Martínez-Flores, I., Salgado, H., et al. (2008) RegulonDB (version 6.0): Gene regulation model of Escherichia coli K-12 beyond transcription, active (experimental) annotated promoters and Textpresso navigation. Nucleic Acids Res., 36, 120-124.

8. De Mey, M., Maertens, J., Lequeux, G. J., Soetaert, W. K. and Vandamme, E. J. (2007) Construction and model-based analysis of a promoter library for E. coli: an indispensable tool for metabolic engineering. 10.1186/1472-6750-7-34.

9. Mitra, A., Kesarwani, A. K., Pal, D. and Nagaraja, V. (2011) WebGeSTer DB-A transcription terminator database. Nucleic Acids Res., 39, 129-135.

10. Rosano, G. L., Ceccarelli, E. A., Neubauer, P., Bruno-Barcena, J. M. and Schweder, T. (2014) Recombinant protein expression in Escherichia coli: advances and challenges. 10.3389/fmicb.2014.00172.

11. Datsenko, K. A. and Wanner, B. L. (2000) One-step inactivation of chromosomal genes in Escherichia coli K-12 using PCR products. Proc. Natl. Acad. Sci., 97(12), 6640-6645.

12. Kuhlman, T. E. and Cox, E. C. (2010) Site-specific chromosomal integration of large synthetic constructs. Nucleic Acids Res.

13. Zhao, D., Yuan, S., Xiong, B., Sun, H., Ye, L., Li,J., Zhang, X. and Bi, C. (2016) Development of a fast and easy method for Escherichia coli genome editing with CRISPR/Cas9. Microb Cell Fact, 15, 205.

14. Stringer, A. M., Singh, N., Yermakova, A., Petrone, B. L., Amarasinghe, J. J., Reyes-Diaz, L., Mantis, N. J. and Wade, J. T. (2012) FRUIT, a Scar-Free System for Targeted Chromosomal Mutagenesis, Epitope Tagging, and Promoter Replacement in Escherichia coli and Salmonella enterica. PLoS One, 7.

15. Ronda, C., Ebdrup Pedersen, L., A Sommer, M. O. and Toftgaard Nielsen, A. (2015) CRMAGE: CRISPR Optimized MAGE Recombineering OPEN. Nat. Publ. Gr., 10.1038/srep19452.

16. Li, Y., Lin, Z., Huang, C., Zhang, Y., Wang, Z., Tang, Y. jie, Chen, T. and Zhao, X. (2015) Metabolic engineering of Escherichia coli using CRISPR-Cas9 meditated genome editing. Metab. Eng., 31, 13-21.

17. Snoeck, N., De Mol, M. L., Van Herpe, D., Goormans, A., Maryns, I., Coussement, P., Peters, G., Beauprez, J., De Maeseneire, S. L. and Soetaert, W. (2018) Serine Integrase Recombinational Engineering (SIRE): A versatile toolbox for genome editing. Biotechnol. Bioeng., 10.1002/bit.26854.

18. Friehs, K. (2004) Plasmid copy number and plasmid stability. Adv. Biochem. Eng. Biotechnol., 86,47-82.

19. Valens, M., Penaud, S., Rossignol, M., Cornet, F. and Boccard, F. (2004) Macrodomain organization of the Escherichia coli chromosome. EMBO J., 23,4330-4341.

20. Sobetzko, P., Glinkowska, M., Travers, A. and Muskhelishvili, G. (2013) DNA thermodynamic stability and supercoil dynamics determine the gene expression program during the bacterial growth cycle. Mol. Biosyst., 9,1643-1651.

21. Peter, B. J., Arsuaga, J., Breier, A. M., Khodursky, A. B., Brown, P .O. and Cozzarelli, N. R. (2004) Genomic transcriptional response to loss of chromosomal supercoiling in Escherichia coli. Genome Biol., 5, R87.

22. Ma, J. and Wang, M. D. (2016) DNA supercoiling during transcription. Biophys. Rev., 8,75-87.

23. Rui, S. and Tse-Dinh, Y.-C. (2003) Topoisomerase function during bacterial responses to environmental challenge. Front. Biosci., 8, d256-63.

24. Cagliero, C. and Jin, D. J. (2013) Dissociation and re-association of RNA polymerase with DNA during osmotic stress response in Escherichia coli. Nucleic Acids Res., 41,315-326.

25. Jeong, K. S., Ahn, J. and Khodursky, A. B. (2004) Spatial patterns of transcriptional activity in the chromosome of Escherichia coli. Genome Biol., 5, R86.

26. Cagliero, C., Grand, R.S., Jones, M. B., Jin, D. J. and O'Sullivan, J. M. (2013) Genome conformation capture reveals that the Escherichia coli chromosome is organized by replication and transcription. Nucleic Acids Res., 41,6058-6071.

27. Dillon, S. C. and Dorman, C. J. (2010) Bacterial nucleoid-associated proteins, nucleoid structure and gene expression. Nat. Rev. Microbiol., 10.1038/nrmicro2261.

28. Vora, T., Hottes, A. K. and Tavazoie, S. (2009) Protein Occupancy Landscape of a Bacterial Genome. Mol. Cell, 35,247-253.

29. Jin, D. J. and Cabrera, J. E. (2006) Coupling the distribution of RNA polymerase to global gene regulation and the dynamic structure of the bacterial nucleoid in Escherichia coli. J. Struct. Biol., 156,284-291.

30. Van Hove, B., Love, A. M., Ajikumar, P. K. and De Mey, M. (2016) Programming Biology: Expanding the Toolset for the Engineering of Transcription. In Glieder, A., Kubicek, C. P., Mattanovich, D., Wiltschi,B., Sauer, M. (eds), Synthetic Biology. Springer, pp. 1-64.

31. Espeli, O., Mercier, R. and Boccard, F. (2008) DNA dynamics vary according to macrodomain topography in the E. coli chromosome. Mol. Microbiol., 68,1418-1427.

32. Sousa, C., de Lorenzo, V. and Cebolla, A. (1997) Modulation of gene expression through chromosomal positioning in Escherichia coli. Microbiology, 143,2071-8.

33. Block, D. H. S., Hussein, R., Liang, L. W. and Lim, H. N. (2012) Regulatory consequences of gene translocation in bacteria. Nucleic Acids Res., 40,8979-8992.

34. Englaender, J. A., Jones, J. A., Cress, B. F., Kuhlman, T. E., Linhardt, R. J. and Ko, M. A. G. (2017) Effect of Genomic Integration Location on Heterologous Protein Expression and Metabolic Engineering in E. coli. Synth. Biol., 10.1021/acssynbio.6b00350.

35. Urtecho, G., Tripp, A. D., Insigne, K., Kim, H. and Kosuri, S. (2018) Systematic Dissection of Sequence Elements Controlling σ70 Promoters Using a Genomically-Encoded Multiplexed Reporter Assay in E. coli. Biochemistry, 10.1021/acs.biochem.7b01069.

36. Bryant, J. A., Sellars, L. E., Busby, S. J. W. and Lee, D. J. (2014) Chromosome position effects on gene expression in Escherichia coli K-12. Nucleic Acids Res., 42,11383-11392.

37. Colloms, S. D., Merrick, C. A., Olorunniji, F. J., Stark, W. M., Smith, M. C. M., Osbourn, A., Keasling, J. D. and Rosser, S. J. (2014) Rapid metabolic pathway assembly and modification using serine integrase site-specific recombination. Nucleic Acids Res., 42, e23.

38. Pédelacq, J. D., Cabantous, S., Tran, T., Terwilliger, T. C. and Waldo, G. S. (2006) Engineering and characterization of a superfolder green fluorescent protein. Nat. Biotechnol., 24,79-88.

39. Shcherbo, D., Murphy, C. S., Ermakova, G. V., Solovieva, E. A., Chepurnykh, T. V., Shcheglov, A. S., Verkhusha, V. V., Pletnev, V. Z., Hazelwood, K. L., Roche, P. M., et al. (2009) Far-red fluorescent tags for protein imaging in living tissues. Biochem. J., 418,567-574.

40. Shaner, N. C., Campbell, R. E., Steinbach, P. A., Giepmans, B. N. G., Palmer, A. E. and Tsien, R. Y. (2004) Improved monomeric red, orange and yellow fluorescent proteins derived from Discosoma sp. red fluorescent protein. Nat. Biotechnol., 22,1567-1572.

41. Davis, J. H., Rubin, A. J. and Sauer, R. T. (2011) Design, construction and characterization of a set of insulated bacterial promoters. Nucleic Acids Res., 39,1131-1141.

42. Cambray, G., Guimaraes, J. C., Mutalik, V. K., Lam, C., Mai, Q. A., Thimmaiah, T., Carothers, J. M., Arkin, A. P. and Endy, D. (2013) Measurement and modeling of intrinsic transcription terminators. Nucleic Acids Res., 41,5139-5148.

43. Engler, C., Gruetzner, R., Kandzia, R. and Marillonnet, S. (2009) Golden gate shuffling: a one-pot DNA shuffling method based on type Ils restriction enzymes. PLoS One, 4, e5553.

44. Ceroni, F., Boo, A., Furini, S., Gorochowski, T. E., Borkowski, O., Ladak, Y. N., Awan, A. R., Gilbert, C., Stan, G. B. and Ellis, T. (2018) Burden-driven feedback control of gene expression. Nat. Methods, 15,387-393.

45. Bertani, G. (1951) Studies on lysogenesis. I. The mode of phage liberation by lysogenic Escherichia coli. J. Bacteriol., 62,293-300.

46. Kahm, M., Hasenbrink, G., Lichtenberg-Fraté, H., Ludwig, J. and Kschischo, M. (2010) Grofit: Fitting biological growth curves. J. Stat. Softw., 33.

47. Singh, M., Yadav, A., Ma, X. and Amoah, E. (2010) Plasmid DNA Transformation in Escherichia Coli: Effect of Heat Shock Temperature, Duration, and Cold Incubation of CaCl2 Treated Cells. Shock, 6,561-568.

48. Phosphate-buffered saline (PBS) (2006) Cold Spring Harb. Protoc., 10.1101/pdb.rec8247.

49. Raghavan, R., Groisman, E. A. and Ochman, H. (2011) Genome-wide detection of novel regulatory RNAs in E . coli. 10.1101/gr.119370.110.21.

50. Hershberg, R. (2001) PromEC: An updated database of Escherichia coli mRNA promoters with experimentally identified transcriptional start sites. Nucleic Acids Res., 29, 277-0.

51. Rudd, K. E. (1999) Novel intergenic repeats of Escherichia coli K-12. Res. Microbiol., 150, 653-664.

52. Yang, Y. T., Bennett, G. N. and San, K. Y. (1999) Effect of inactivation of nuo and ackA-pta on redistribution of metabolic fluxes in Escherichia coli. Biotechnol. Bioeng., 65, 291-297.

53. Zhang, Z., Yen, M. R. and Saier, M. H. (2010) Precise excision of IS5 from the intergenic region between the fucPIK and the fucAO operons and mutational control of fucPIK operon expression in Escherichia coli. J. Bacteriol., 192, 2013-2019.

54. Kim, S. M., Choi, B. Y., Ryu, Y. S., Jung, S. H., Park, J. M., Kim, G. H. and Lee, S. K. (2015) Simultaneous utilization of glucose and xylose via novel mechanisms in engineered Escherichia coli. Metab. Eng., 30, 141-148.

55. Overmars, L., Van Hijum, S. A. F. T., Siezen, R. J. and Francke, C. (2015) CiVi: Circular genome visualization with unique features to analyze sequence elements. Bioinformatics, 31, 2867-2869.

56. Casini, A., Christodoulou, G., Freemont, P. S., Baldwin, G. S., Ellis, T. and MacDonald, J. T. (2014) R2oDNA Designer: Computational Design of Biologically Neutral Synthetic DNA Sequences. ACS Synth. Biol., 3, 525-528.

57. Lou, C., Stanton, B., Chen, Y. J., Munsky, B. and Voigt, C. A. (2012) Ribozyme-based insulator parts buffer synthetic circuits from genetic context. Nat. Biotechnol., 30, 1137-1142.

58. Rhodius, V. A., Mutalik, V. K. and Gross, C. A. (2012) Predicting the strength of UP-elements and full-length E. coli σ e promoters. Nucleic Acids Res., 40, 2907-2924.

59. Lal, A., Dhar, A., Trostel, A., Kouzine, F., Seshasayee, A. S. N. and Adhya, S. (2016) Genome scale patterns of supercoiling in a bacterial chromosome. Nat. Commun., 7.

60. Chong, S., Chen, C., Ge, H. and Xie, X. S. (2014) Mechanism of transcriptional bursting in bacteria. Cell, 158, 314-326.

61. Ceroni, F., Algar, R., Stan, G. B. and Ellis, T. (2015) Quantifying cellular capacity identifies gene expression designs with reduced burden. Nat. Methods, 12, 415-418.

62. Scholz, S., Diao, R., Wolfe, M., Fivenson, E., Lin, X., Freddolino, P. (2019) High-resolution mapping of the Escherichia coli chromosome reveals positions of high and low transcription. Cell Systems, 8, 1-14.

63. Gietz R D, Schiestl R H. High-efficiency yeast transformation using the LiAc/SS carrier DNA/PEG method. Nat Protoc. 2008;2(1):31-35.

1. Method to determine the expression stability of a chromosomal location in an isolated cell, said method comprising:
providing an isolated cell to be transformed;
chromosomally integrating a marker cassette in said cell at said chromosomal location;
imposing a burden upon said cell comprising said marker cassette;
determining the expression of the marker with and without said burden, wherein i) a stable location is not influenced by said burden or ii) a sensitive location shows a reduced expression due to said burden;
preferably scoring said expression stability of said chromosomal location of said cell.

2. Method to determine relative expression stability of a chromosomal location in an isolated cell, said chromosomal location providing a tuneable integration location for production of a desired metabolite, said method comprising the following steps:
providing an isolated cell;
chromosomally integrating a marker cassette in said cell at said chromosomal location;
imposing a burden upon said cell comprising said marker cassette at said chromosomal location;
measuring the influence of the imposed burden in comparison with said cell i) with the integrated marker but without the burden imposed; ii) without the integrated marker but under the same imposed burden and/or iii) in comparison with an isolated cell of the same organism with another integration location of said marker cassette and under the same burden, by determining the expression of the marker;
preferably scoring the performance of said integration location(s).

3. Method to produce stable expression transformants of an isolated cell, said method comprising:
a) i) providing an isolated cell;
ii) chromosomally integrating in said cell a marker cassette;
iii) imposing a burden upon said cell comprising said marker;
iv) measuring the influence of the imposed burden in comparison with said cell without said burden;
v) repeating steps a) i) to iv) for several chromosomal integration locations;
vi) selecting the cells with a good or unchanged production of the marker under burden thereby obtaining or identifying the desired stable expression location(s);
b) providing untransformed isolated cells
transforming said untransformed cells with a desired gene, genetic cassette or set of genes at the location obtained from step a) vi).

4. Method to produce a burden repressible transformant of an isolated cell, said method comprising:
a) i) providing an isolated cell;
ii) chromosomally integrating in said cell a marker cassette;
iii) imposing a burden upon said cell comprising said marker;
iv) measuring the influence of the imposed burden in comparison with said cell without said burden;
v) repeating steps a) i) to iv) for several chromosomal integration locations;
vi) selecting the cells with a reduced production of the marker under burden thereby obtaining or identifying the desired burden repressible location(s);
b) providing untransformed isolated cells
transforming said untransformed cells with a desired heterologous gene, genetic cassette or set of genes at said location obtained from step a) vi).

5. Method according to any one of claims 1 to 4, wherein said marker cassette is integrated at a non-essential gene chromosomal locus or at an intergenic region, preferably avoiding regulatory leader sequences, regions that contain promoters, 5′-UTRs, 3′-UTRs, transcription terminators, sigma factors, enhancers or silencers.

6. The method according to any one of claims 1 to 5 wherein the marker cassette is flanked with insulating DNA sequences, wherein said insulating DNA sequences are preferably transcription terminators.

7. The method according to any one of claims 1 to 6 wherein the marker cassette is an antibiotic resistance cassette, a colorant cassette or a fluorescent cassette.

8. The method according to any one of claims 1 to 7 wherein the imposed burden is a chemical, physical or genetic/expression burden, preferably the genetic/expression burden is the expression of a plasmid, preferably a chemical burden is a high concentration of at least one medium component, preferably a physical burden is a non-natural pH, a shear stress condition, a non-natural temperature or cold or heat stress, non-natural pressure conditions, and/or osmotic pressure.

9. The method according to any one of claims 2 and 5 to 8, wherein the tuneable transformation is a stable transformation.

10. The method according to any one of claims 2 and 5 to 8, wherein the tuneable transformation is a relative repression of the integrated marker or heterologous gene under burden.

11. Method for the production of a bioproduct using a genetically modified host cell, the method comprising the steps of:
providing a host cell, which has been genetically modified, such, that at least said cell is able to produce the bioproduct wherein the unmodified host cell is not able to produce the bioproduct, due to the introduction of at least one heterologous gene, encoding the bioproduct or an intermediate thereof, which is expressed in the host cell;
cultivating and/or growing said genetically modified host cell in a cultivation medium enabling to production of the bioproduct thereby producing the bioproduct obtainable from the medium the host cell is cultivated in;
characterised in that the heterologous gene is introduced at a chromosomal location obtainable from the method of any one of claims 1 to 10.

12. The method according to any one of claims 1 to 11 wherein the cell is a cell of a microorganism, plant, or animal, preferably said microorganism is a bacterium, fungus or a yeast, preferably said plant is a rice, cotton, rapeseed, soy, maize or corn plant, preferably said animal is an insect, fish, bird or mammal.

13. Method to produce stable transformants of E. coli expressing a desired gene, genetic cassette and/or set of genes, said method comprising the following steps:
providing E. coli cells,
transforming said cells by the introduction of a desired heterologous gene, genetic cassette or set of genes at at least one intergenic position chosen from the list of E. coli genomic intergenic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ.

14. Method to produce burden repressible transformants of E. coli expressing a desired heterologous gene, genetic cassette and/or set of genes comprising the following steps:
providing E. coli cells,
transforming said cells by the introduction of a desired heterologous gene, genetic cassette and/or set of genes at at least one intergenic position chosen from the list of E. coli genomic intergenic locations djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

15. Method to produce a desired bioproduct or metabolite by E.coli, said method comprising the following steps:
providing E. coli cells,
providing a bioproduct or metabolite production heterologous gene, genetic cassette and/or set of genes
transforming said cells by introduction of said desired heterologous gene, genetic cassette or set of genes at at least one intergenic positions chosen from the list of E. coli genomic locations ypjC_ileY, yjip_yjiR, ykgH_betA, thrW_ykfN, ykgA_ykgQ, ileY_ygaQ, ybfC_ybfQ, yeeJ_yeeL, ymgF_ycgH and cspF_quuQ
growing said cells in a medium permissive for the production of the desired bioproduct or metabolite.

16. Method to produce a desired bioproduct or metabolite by E. coli, said method comprising the following steps:
providing E. coli cells,
providing a bioproduct or metabolite production heterologous gene, genetic cassette and/or set of genes
transforming said cells with said desired heterologous gene, genetic cassette and/or set of genes at at least one intergenic positions chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH and cspF_quuQ, djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT;
growing said cells in a medium permissive for the production of the desired bioproduct or metabolite.

17. Method according to any one of claim 11, 12, 15 or 16, wherein said bioproduct is an oligosaccharide, preferably sialic acid or sialylated, fucosylated, galactosylated oligosaccharide, more preferably a human milk oligosaccharide.

18. Use of E. coli chromosome position for tuneable transformation by introduction of at least one desired heterologous gene at at least one intergenic chromosome location, wherein said at least one intergenic chromosome location is chosen from the list of E. coli genomic locations yjip_yjiR, ykgH_betA, thrW_ykfN, ykgA_ykgQ, ileY_ygaQ, ybfC_ybfQ, yeeJ_yeeL, ymgF_ycgH, cspF_quuQ, djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

19. An E. coli cell transformed by the introduction of at least one heterologous gene at at least one intergenic location chosen from the list of E. coli genomic intergenic locations yjip_yjiR, ykgH_betA, thrW_ykfN, ykgA_ykgQ, ileY_ygaQ, ybfC_ybfQ, yeeJ_yeeL, ymgF_ycgH and cspF_quu, djlA_yabP, frwA_frwC, glpD_yzgL, malts_yjbl, frvA_rhaM, yhiM_yhiN, yqaB_argQ, ygcE_queE, ybiJ_ybil, ybfK_kdpE, rseX_yedS, udk_yegE and tyrV_tyrT.

20. An E. coli cell transformed by the introduction of heterologous gene to produce an oligosaccharide, said cell transformed with at least one gene, genetic cassette or set of genes at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH, cspF_quuQ djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

21. An E. coli cell according to claim 20, wherein said oligosaccharide contains monosaccharides selected from the group comprising: glucose, galactose, N-acetylglucosamine, glucosamine, mannose, xylose, N-acetylmannosamine, N-acetylneureminic acid, N-glycolylneuraminic acid, a sialic acid, N-acetylgalactosamine, galactosamine, fucose, rhamnose, glucuronic acid, gluconic acid, fructose, polyols.

22. An E. coli cell transformed by the introduction of at least one heterologous gene to produce a sialic acid pathway, N-acetylglucosamine carbohydrate pathway, sialylation pathway, or fucosylation pathway or galactosylation pathway, said cell transformed at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH cspF_quuQ, djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

23. Method to produce a sialic acid or sialylated, fucosylated, galactosylated oligosaccharide with a cell according to any one of claims 20 to 22, respectively.

24. An E. coli cell transformed to produce a human milk oligosaccharide pathway, said cell transformed by the introduction of at least one gene at at least one intergenic location chosen from the list of E. coli genomic locations ypjC_ileY, yjip_yjiR, ykgH_betA, thrW_ykfN, ykgA_ykgQ, dadX_cvrA, ileY_ygaQ, ybfC_ybfQ, yeeJ_yeeL, ymgF_ycgH, cspF_quuQ, djlA_yabP, frwA_frwC, glpD_yzgL, malts_yjbl, sibD_sibE, frvA_rhaM, yhiM_yhiN, yqaB_argQ, yffL_yffM, ygcE_queE, ybiJ_ybil, ybfK_kdpE, rseX_yedS, udk_yegE and tyrV_tyrT.

25. Method to produce a human milk oligosaccharide with the cell according to claim 24.

26. Method for the production of a bioproduct using a genetically modified host cell according to any one of claim 18 to 22, or 24.

27. Method according to claim 26, wherein said bioproduct is an oligosaccharide, preferably a human milk oligosaccharide.

28. Use of a host cell for the production of an oligosaccharide wherein said host cell expresses a heterologous protein which heterologous protein's coding sequence was introduced at a location of said host cell, said location being defined by any one of the methods of claim 1 to 12.
